SEQUENCE LISTING (62 sequences)

SEQ ID NO: 1 - DNA, 2202 bp, synthetic construct
  source 1..2202: mol_type = other DNA
tggaagtttcactgctgcttcgtcaatactcattgcctttatattttcctcattcactctcttcccattctcatctctctctctctacctttctctcacacaaagagttttttcttttaagacaaaaactctttcaaggttttaaatctttgaaaaaattctctaaacctaaaagtgatcaatgaagattcggaacagccctagttttcatgggggtagtggttacagtgtctttagagctcgtaacttaacattcaagaaagttgtgaagaaagtgatgagagaccagtcaaataatcaattcatgattcagatggagaacatgataagaagaattgtccgagaggagatacagcgtagtcttcaaccttttctctcttcatcatgcgtctcaatggagcgatctcgctcagaaacgccatcgtctcgctcacgattgaagctgtgcttcataaactcgccgccatcatcgatattcacggggtccaagatcgaagctgaggatggttctccgcttgtgatcgagctcgtggacgccaccacaaacactctagttagtacgggaccgttctcgtcttctcgggtcgagctcgtgccgctgaacgctgatttcacggaagaaagctggaccgttgagggatttaatcggaatattctcacgcaacgtgaagggaaacgtccgttgctcactggagacctaacggtgatgcttaaaaacggtgttggagttataaccggagatatagctttctcggataactcgagctggactaggagtcggaagttccggttaggtgctaagttgaccggagatggagccgtggaggcgagaagtgaagcttttggatgtagagaccaacgaggagaatcttataaaaaacatcatcctccgtgccccagtgatgaggtttggagactagagaaaatcgcgaaagatggagtttcggcaacgcgtttggctgaacgcaagattttaaccgtcaaggattttcgccgtttgtatactgtaaatcgaaatgagttacacaacataattggtgcaggggtctcaaagaaaacatggaacacaattgtatcacatgccatggattgcgttttggacgaaacagagtgttacatttacaatgcaaacactccgggcgtaacacttctcttcaactctgtttatgagttgataagagtgtcattcaatggcaacgatatccaaaaccttgatcagccaattctagaccaattaaaggccgaagcttatcaaaaccttaaccgcattacagcggttaacgataggacctttgtgggtcatccacaaaggtccttacagtgcccgcaagatcctggatttgtcgtaacatgttctggatcgcagcacatcgactttcaaggaagtttggatccatcaagctcttcgatggctctttgccacaaagcttcaagctcaacggtccaccctgatgtcctgatgagttttgataactcatcaaccgcgaggtttcatatcgacaaaaagttcttaccgactttcggaaacagcttcaaagtaagtgaactcgatcaagtacacggaaaatcacaaactgttgtgacaaaaggttgtatagagaataacgaggaggatgagaacgcgttttcttatcatcaccatgatgacatgacctcaagctggtcacctggtacgcaccaagccgttgaaacgatgtttcttaccgtgtctgagacggaagaagctggaatgttcgatgttcattttgcaaacgttaatttgggatctccaagagccaggtggtgtaaggttaaggcagctttcaaggttagggcagcttttaaggaagtccggagacacacaactgccagaaatccgagggaaggcttgtaagaactcttgcctacctattaaaatcagcggtttcttgcgttgcacgataaatgtgtatatatatttgtgaatagggttagataaatggatcccggccattgagttacggggattagcctttgcgtatatcaaagtatttttttccctgggtaagtatagctgtggcacagggtttgtaagttgtaaccatatgccgtataagaatcagaggtttcttgttgtacacgaaagctatgtataaataaaagctatgtatatatgtttttttctaccataacttttcaataatggatctcggctattgattgttctagaccaatatgatacat

SEQ ID NO: 2 - DNA, 3823 bp, synthetic construct
  source 1..3823: mol_type = other DNA
tctctcgcgacggatcttcttaggtctctctcttctcctccttttccccactttttcaaaatcagcaaattttggattttcagaatttgagtgaacttaaagtagaagacgaattgtctgaattcgaattttaagaaaaatttgcagagatgaggagttttttcggtagaggagggaaggattcaccggctgattctgcttctccgtcaccgagatcatacccttcgacgtctccggcgtcttcgtctgctgtgacgggaccaccgagacctattcgtttagtgtattgtgatgagaaagggaagtttcgtatggaccctgaagctgttgctactttgcaactcgttaaggagccaattggtgttgtttcggtttgtggtagggctcgtcaaggaaagagctttattttgaatcagcttcttggacgcagtaatgggtttcaagtagcatcaacacacaagccgtgtaccaaggggctttggttgtggagttctcctataaaaagaacagctcttgacggaactgaatacaatcttttgttattagatagtgaaggaattgatgcttacgaccaaactggtacctacagcactcaaatattctcattagctgttcttttgtcaagcatgttcgtgtataatcagatgggaggcattgacgaagcttcactggatcgcctttctcttgtcactcaaatgacaaagcatatccgcgtaaaagcttctggagggacgagctcacgttctgaacttgggcaattttctcccatctttgtctggctgttgagggacttttatttggatctagtggaagataatcggaagattagcccacgtgactatttagaaattgccttaaggccagttcaaggttcgggaggggatattggtgctaaaaacgagatccgagattcaatccgtgctctttttccggacagagagtgctttacccttgtaaggcctctgaacaatgagaaggacctgcagagactggatcaaatttcgttggaaaaattgagacctgaattcggtgctggcctggatgcatttaccaagtttgtttttgagaagacgaggcccaagcaattagggggtactgtgatgactggccctattcttgtcggtattacacagtcttatctggatgctttgaataatggtgccgtgccaacaataacctcatcttggcagagtgtcgaagaaactgagtgtcgaagagcatatgattctggtgtagaagcctatatggctgcctttgaccaatcaaaagctccggaagaaggtgcactgagggaagaacatgaagaagcagttcgaaaagccttggctatatttaactctaatgctgtagggaatggttcagcaagaaaaaaattcgaggatcttctccacaaggacttaaagaaaaagtttgaggattacaagaagaacgcttttatggaggcagatttgcgatgtacgagtactatccagcgtatggaaaagcagcttagagcagcttgccatgcctctaatgccaatatggataatgttgtcaaggtcctagaagctcgtttggcagaatatgaggcatcatgccatggcccggggaaatggcagaaactttctgtatttttgcaacaaagcttggaaggacctatatatgatctcaccaaaaggcttatagatagcatcgctatagagaagaattcccttgcaatgaaatttcgttctgttgaagatgcgatgaaacatttaaaacagcagttggatgatagtgagagatacaagttggaataccagaaacgttatgatgaatccaacaatgacaagaagaagctggaagatatatacagagagcgcataactaaactacagggggagaatagttcgctgaacgagcgatgctctacgttggttaaaaccgtggaatctaaaaaagaagaaatcaaagaatggattagaaattatgaccagattgttttgaagcagaaagctgttcaagaacagcttagttctgaaatggaagtccttaggacaagaagtactacttctgaagctagggttgctgcagctagagaacaagccaaatctgctgcagaagagaccaaggaatggaaaaggaagtatgattatgctgtgggagaggctagatctgctcttcaaaaggctgcttcagtgcaagaacgttctggcaaggaaacacagttgagggaagatgctcttagggaggaatttagtatcactttagctaataaggatgaagaaattacggagaaggctacaaaacttgagaaggcagagcaatctctaacggttctaagatcagatttgaaggtggctgagtcaaaacttgaaagctttgaggtagagttagcatcactaagattaacattaagtgaaatgactgataagttggacagtgccaacaaaaaggctctagcatatgaaaaggaggctaataagttggagcaagagaaaatccgtatggaacaaaagtatcgatctgagtttcaacgcttcgatgaagtcaaagagagatgtaaggcagctgaaatagaagctaaacgcgctactgaactggcagacaaagctcgaactgatgctgtcacgtcgcaaaaggagaaaagtgagagtcagaggttggcgatggaaagacttgctcagatcgaacgagctgagaggcaagtcgaaaacttagagagacagaagactgacttggaggatgaactggacagacttcgtgtgtctgagatggaggctgtctccaaagttacaatcttagaagcaagagttgaagaacgagagaaagaaatcgggtcattgataaaggaaaccaacgcacagagggctcataacgtgaagtcacttgaaaagctgttggatgaagagcgtaaggctcatatagctgcaaatcgaagagccgaagctctttctcttgagctgcaagctgcacaggcacacgttgataatcttcagcaagaattagctcaagcaaggttaaaagaaaccgcacttgacaacaaaatcagagctgcgagttcctcacatgggaagcggagtagatttgaagatgttgttgatatggacattggagaaggaagtgatagaatcttgaggactaataaacgagctcgaagcacgagaggagatgatcacggtcctactgatgaaggcgacgaggactttcaaagtcatcaagacaatggcgaggaggaagaagaagaagattacaggaagcttactgtgcagaatctaaagcatgagctgaccaagtacgattgtggacatttgttattgaacagaggccatcaaaacaagaaagagattcttgcgttgtatgaagctcatgttcttcccaagaaagctttagcgagagaggaagagagaaagaaacagagagaagtcacttcttcttagaggacaagactctgtacagagtaaggagataaacatgtttaatctcaaaaggtttcgatctctagagatttagtaagagttggaattagaaaaggctcgtattgtttctctctttttatcggttttaccggtttacctggtactgcagaaagtaaaccgaattagccatttgtattgaatctgagggtttaggtttttttatgagtgttttttttttgtgtgtttggtatttaatttgtgtggagtttgagtcggtttggtgttttgactttttgatttttagattcaagtctttttcagaattctgttactcgtttgagatcgaccgtttgctttgaattttactcttcaatcctaagtcagcagtattgactaattgcgtttaaaggaataattctaagattatgttaacttggtgataggct

SEQ ID NO: 3 - DNA, 2120 bp, synthetic construct
  source 1..2120: mol_type = other DNA
ccaaaaaaaaacagaaagagcaatctctctatactacaaacaccaaagtttaaaattaaacaaaatcttcaagaaacactttacgaatttctgcaatggcttcacttcaattttcttctcagtttctgggctcaaacactaaaacacacagctctatcatttccatctctcgtagttactctccaactccattcactagattctcccgcaagaagtatgagtcatgttcgatgtctatgaatggttgtgatggagatttcaagacgccacttggtacagtggagacaaggactatgactgctgttttatctccggcagccgccactgaaaggctaatctccgccgtctctgaactcaaatctcaacctccgtcgttttcctccggcgtcgttcggttacaggttccaattgaccagcaaatcggagcaattgattggcttcaagcccagaatgagattcagcctcgctgtttcttctctcgtcgcagtgacgttggtcgtcccgatcttcttctcgatctagctaacgagaacggaaacggaaacggaaacggaacagtgtcatctgatcgtaatctggttagcgttgctggtatcggctctgcagttttcttccgtgaccttgatcctttctctcatgacgattggagatccatcagaaggtttttgtcttcaacgtcacctctgattcgtgcctatggtggtatgcgttttgatcctaatggcaagatcgctgttgaatgggaaccttttggtgcattttacttttcagtccctcaggttgagtttaatgagtttggtggaagttcaatgttggctgcaactattgcttgggatgatgaactctcttggactctggaaaatgctattgaagcactccaggagactatgcttcaagtttcttctgttgtaatgaagttgagaaacagatctttaggagtatctgttttaagcaagaatcatgttcctaccaaaggagcttatttccctgctgtagagaaggctttagagatgattaaccagaaaagttcaccccttaacaaggttgttcttgctcgtaacagcaggataattacggataccgacattgatcccattgcttggctagcacagttacagcgtgaagggcatgatgcatatcagttctgtcttcaaccacctggtgcaccagcttttatcggaaacacgcctgagagactattccaaaggactcaattaggtgtctgcagtgaagctttggctgcaactaggcctagagctgcttctagtgctcgtgatatggagatagagcgtgacttactaaccagtccgaaagacgacctcgagttctctatcgtacgagagaatataagagaaaagttaaacggtatatgtgacagagttgttgtcaagcctcaaaaaactgtgaggaagcttgcaagagtgcaacatctatattctcaattggcagggagacttacgaaggaagatgatgagtataaaatattggctgctctgcatccaactccagctgtttgtgggcttccagcagaagaagcaaggcttttgattaaggagatagaatcattcgatagaggaatgtatgcgggacctattggattttttggtggcgaggagagtgaatttgcagtcgggatcagatcagctctagtcgaaaagggtcttggggcattgatctatgcggggacagggatagtagctggaagtgacccatcttcagagtggaatgagcttgatcttaagatatctcaggtacgagcttttgtccagaaaatgtttagtgacatcatggttctctgttaccaaaatcctaatttttattctctcttttgttgttgtttttgcagttcaccaagtcaattgaatatgaagcaacaacatctctacaggcgattaattgaagaaagagtaacatttgtatttgattgttttgtttgtatgggggataaggggttctcacaataagaaagcaatgttgtctctcttgtaaattaaaaaagaaatgctttaatttgttaatgggccgagccttttcgggttgtaactagggcaggcctatcatgaatgttcataggcctagctgttatgattgttaatgagcttctatagtgtttttcttca

SEQ ID NO: 4 - DNA, 23 bp, synthetic construct
  source 1..23: mol_type = other DNA
caccatgaagattcggaacagcc

SEQ ID NO: 5 - DNA, 23 bp, synthetic construct
  source 1..23: mol_type = other DNA
ttacaagccttccctcggatttc

SEQ ID NO: 6 - DNA, 39 bp, synthetic construct
  source 1..39: mol_type = other DNA
cgcggccgcccccttcaccaacagcatccgtttttataa

SEQ ID NO: 7 - DNA, 40 bp, synthetic construct
  source 1..40: mol_type = other DNA
gggctgttccgaatcttcatctccggcgaactttttttat

SEQ ID NO: 8 - DNA, 29 bp, synthetic construct
  source 1..29: mol_type = other DNA
caccatgaattcgacatcgacacattttg

SEQ ID NO: 9 - DNA, 20 bp, synthetic construct
  source 1..20: mol_type = other DNA
cgttggttcacgatgtcgag

SEQ ID NO: 10 - DNA, 22 bp, synthetic construct
  source 1..22: mol_type = other DNA
ggttacaagacaaggttcactc

SEQ ID NO: 11 - DNA, 22 bp, synthetic construct
  source 1..22: mol_type = other DNA
cattcaggaccaaactcttcag

SEQ ID NO: 12 - DNA, 21 bp, synthetic construct
  source 1..21: mol_type = other DNA
tcgtggacgccaccacaaaca

SEQ ID NO: 13 - DNA, 20 bp, synthetic construct
  source 1..20: mol_type = other DNA
tcagcgttcagcggcacgag

SEQ ID NO: 14 - DNA, 25 bp, synthetic construct
  source 1..25: mol_type = other DNA
acttactaaccagtccgaaagacga

SEQ ID NO: 15 - DNA, 25 bp, synthetic construct
  source 1..25: mol_type = other DNA
acaacaactctgtcacatataccgt

SEQ ID NO: 16 - DNA, 22 bp, synthetic construct
  source 1..22: mol_type = other DNA
tcgctttccaaccatctttctc

SEQ ID NO: 17 - DNA, 21 bp, synthetic construct
  source 1..21: mol_type = other DNA
ctactcctcacagccatttcc

SEQ ID NO: 18 - DNA, 20 bp, synthetic construct
  source 1..20: mol_type = other DNA
atcttctccgccgtcattcc

SEQ ID NO: 19 - DNA, 20 bp, synthetic construct
  source 1..20: mol_type = other DNA
cacgtggcagaagttgtgtg

SEQ ID NO: 20 - DNA, 24 bp, synthetic construct
  source 1..24: mol_type = other DNA
gttccctaggagttactataggtg

SEQ ID NO: 21 - DNA, 21 bp, synthetic construct
  source 1..21: mol_type = other DNA
tttcggtggatttctcgatgg

SEQ ID NO: 22 - DNA, 21 bp, synthetic construct
  source 1..21: mol_type = other DNA
ctagtctttgagggtcttgtg

SEQ ID NO: 23 - DNA, 21 bp, synthetic construct
  source 1..21: mol_type = other DNA
tgagcattggttcactaacag

SEQ ID NO: 24 - DNA, 20 bp, synthetic construct
  source 1..20: mol_type = other DNA
acgggaaaggactggaagag

SEQ ID NO: 25 - DNA, 20 bp, synthetic construct
  source 1..20: mol_type = other DNA
atggttgcggatttccttgg

SEQ ID NO: 26 - DNA, 20 bp, synthetic construct
  source 1..20: mol_type = other DNA
cttgcggttcataacgatgc

SEQ ID NO: 27 - DNA, 20 bp, synthetic construct
  source 1..20: mol_type = other DNA
tagttttgtgctcgggatgc

SEQ ID NO: 28 - DNA, 20 bp, synthetic construct
  source 1..20: mol_type = other DNA
tgttcatcagtgtgctagtg

SEQ ID NO: 29 - DNA, 20 bp, synthetic construct
  source 1..20: mol_type = other DNA
gctgtccttccttctgaatc

SEQ ID NO: 30 - DNA, 19 bp, synthetic construct
  source 1..19: mol_type = other DNA
acgccttcacggtccatac

SEQ ID NO: 31 - DNA, 25 bp, synthetic construct
  source 1..25: mol_type = other DNA
aaacagaaagaaacagagggagtac

SEQ ID NO: 32 - DNA, 23 bp, synthetic construct
  source 1..23: mol_type = other DNA
ccgtttgtagagccataattgca

SEQ ID NO: 33 - DNA, 25 bp, synthetic construct
  source 1..25: mol_type = other DNA
aggttgcctgagtcacagttaagtg

SEQ ID NO: 34 - DNA, 20 bp, synthetic construct
  source 1..20: mol_type = other DNA
ctgcgtggaagtctgtcaaa

SEQ ID NO: 35 - DNA, 20 bp, synthetic construct
  source 1..20: mol_type = other DNA
ctatgccacagggcagtttt

SEQ ID NO: 36 - DNA, 23 bp, synthetic construct
  source 1..23: mol_type = other DNA
ggggtcaatgtacttctagttga

SEQ ID NO: 37 - DNA, 20 bp, synthetic construct
  source 1..20: mol_type = other DNA
aaactagccaaaacagccgt

SEQ ID NO: 38 - DNA, 20 bp, synthetic construct
  source 1..20: mol_type = other DNA
gaagattcggaacagcccta

SEQ ID NO: 39 - DNA, 20 bp, synthetic construct
  source 1..20: mol_type = other DNA
tccgaaaataaaccggaaaa

SEQ ID NO: 40 - DNA, 20 bp, synthetic construct
  source 1..20: mol_type = other DNA
gcagtggaccaaagagcaat

SEQ ID NO: 41 - DNA, 21 bp, synthetic construct
  source 1..21: mol_type = other DNA
caagatcatcacaagcagcga

SEQ ID NO: 42 - DNA, 20 bp, synthetic construct
  source 1..20: mol_type = other DNA
aatgtaaaccgtgggacgag

SEQ ID NO: 43 - DNA, 20 bp, synthetic construct
  source 1..20: mol_type = other DNA
taagaatcggcgaatccatc

SEQ ID NO: 44 - protein, 489 aa, unidentified
MDLKRVLDVVEEEVVDGDEEELASPDAKRRRTLLNSSSSMQEAIGAQYMQRHLPKLEPFLRRVVQEEVHNVLIRHIDSAHRLPLQLKTSSKRYKLQFQGNLPQTLFTGNRVEAENKQPLRIVLTDAVNNQTITSGPLSSMKVELLVLDGDFNADERLEHTEKEFSESIVFEREGKRPLLSGEVIIVLEKGVASVRDISFTDNSSWIRSRKFRLGARMSRASSIEERVQEAVSNPFLVKDHRGEVYKKHHPPALADDVWRLEKIGKDGVFHKKLADFGIHTVQDFLRNLVMDQNGLRGLLGSGMSNKMWESTVEHARECVLDDKLYSYCSGHGIILLFNCIHEIVGVIVGSHCFTLNVLTATQKALVVKLQQDAYKFPNRIVEFKLQSQCAADQSPPMPAGPSCTHIPSLPRDEPNLQDDGLLNPLQQLPLGEALEDVLQLQATSGSHQHHRAEPWITSGFDARDPFDALQFSGSSQPCGLLLSSTGARL

SEQ ID NO: 45 - protein, 516 aa, Sorghum bicolor
MDLKRALDVEEEVVDGDEDELAGCPDAKRRRTFVNSSMQEAIGAQYMQRHLPKLEPFLRRVVQEEVHNVLIRHIDSANRLPLQLKTTSKRYKLQFQGNLPQTLFTGNRVEAENKQPLRLVLTDAATNQTVTSGPLSSMKVELLVLDGDFNADERLEHTEKEFSESVVFEREGKRPLLSGEVIIVLEKGTASIRDISFTDNSSWIRSRKFRLGARMSRASTIEERVQEAVSNPFLVKDHRGEVYKKHHPPALADDVWRLEKIGKDGVFHKKLADFGIHTVQDFLRNLVMDQYGLRSLLGSGMSNKMWESTVEHARECVLDDKLYSYCSGHGIVLLFNCIHEVVGVIVGSHCFTLSALTPTQKALVVKLQQDAYKFPDRIAEFKVQSQSAADQQPPAAAVQAPPAPLPVPAAQMLGLPHGHGVVVQPSAGAPASSHDGHGLLLNPQLLQHHQQQQPQPLSEALEDVLQSASAAHQLNAAEPWFPSFGAGGFDARDPFDVQFSGSQPCGLLLSSTGARL

SEQ ID NO: 46 - protein, 506 aa, Zea mays
MDLKRTLDVEEEVVDGDEDELACCPDAKRRRTFVDSSMQEAIGAQYMQRHLPKLEPFLRRVVQEEVHNVLIRHIDSAHRLPLQLKTSSKRYKLQFQGNLPQTLFTGNRVEAESKQPLRLVLTDATTGQTVASGPMSSMKVELLVLDGDFNADERLVEHSEKEFSESVVFEREGKRPLLSGEVVVVLEKGAASIRDISFTDNSSWIRSRKFRLGARMSRASSIEERVLEAVSNPFLVKDHRGEVYKKHHPPALADDVWRLEKIGKDGVFHKKLADFGIHTVQDFLRNLVMDQYGLRSLLGSGMSNKMWESTVEHARECVLDDRLYSYCSGHGTVLLFNCVYEVVGVLVGGHCFTLSALTPTQKALVGQLQQDAYKFPDRIAEFKVQSQSAGVEQPPAAVESPPSVPGAQVLGLPHGVVQVQPSAGAPSAHDLLLSPRLLQHQQQPLSEAALEDVLQSASAAHQLDAAEPWFPSFGAGGFDARDPFDVQFGGGSQPCGLLLSSTGARL

SEQ ID NO: 47 - protein, 487 aa, Hordeum vulgare
MDLKRVLDIVEEEVVDGDEEELASPDAKRRRTLLNSSSMQEAIGAQYMQKHLPKLEPFLRRVVQEEVHNVLIRHIDSAHRLPLQLKTSSKRYKLQFQGNLPQTLFTGNRVEAENKQPLRIVLTDAVNNQLITSGPLSSMKVELLVLDGDFNADERLEHTEKEFSESIVFEREGKRPLLSGEVVIVLEKGVASVRDISFTDNSSWIRSRKFRLGARMSRASSIEDRVQEAVSNPFLVKDHRGEVYKKHHPPALADDVWRLEKIGKDGVFHKKLADFGIHTVQDFLRNLVMDQYGLRGLLGSGMSNKMWESTVEHARECILDDKIYSYCSGHGIILLFNCIHEIIGVVVGSNCFSLNALTATQKALVVKLQQDAYKFPSRIVEFKVQPQCADQSPTAPAPPGPASTQIPGLAQGEANPHPQEHGLLHQLPLHDAALGLEDVLHQQHRHRHSEPWITNGFDATRDPFDMLQFSGSSQPCGLLLSSTGARL

SEQ ID NO: 48 - protein, 486 aa, Triticum aestivum
MDLKRVLDIVEEEVVDGDEEELASPDAKRRRTLLNSSSMQEAIGAQYMQKHLPKLEPFLRRVVQEEVHNVLIRHIDSAHRLPLQLKTSSKRYKLQFQGNLPQTLFTGNRVEAENKQPLRIVLTDAVNNQLITSGPLSSMKVELLVLDGDFNADERLEHTEKEFSESIVFEREGKRPLLSGEVVIVLEKGVASVRDISFTDNSSWIRSRKFRLGARMSRASSIEERVQEAVSNPFLVKDHRGEVYKKHHPPALADDVWRLEKIGKDGVFHKKLADFGIHTVQDFLRNLVMDQYGLRGLLGSGMSNKMWESTVEHARECVLDDKLYSYCSGHGIILLFNCIHEIIGVVVGSNCFSVNALTATQKALVVKLQQDAYKFPSRIVEFKVQSQSADQSPTAPVPPGPASTQMPGLPPQGGEANPQPQDHGLLPLHDAALGLEDVLHQHHRRHHSEPWITDGFDATRDPFDMLQFSGSSQPCGLLLSSTGARL

SEQ ID NO: 49 - protein, 569 aa, Solanum lycopersicum
MQKTTLTSTRLLVSLPNWTLMVLKRNINQDSDENSEFPPRESKRQHPSNAFFSGLRSYSSPQELARKLEPLIRSWVREEIEQTLQSTLRSSLNDDETSKSRDIQLLFDDVFPSSLFTGSKLSMENRPIKVLLYDASSNQRITSGPLSSAKVDVVVLNGEFNPSNLEDWSEEEFSKMVIRAREGKRPLLTGDLIIQLRDGVGYLGDLSFTDNSSWIKSRTFRLGVKLNNRSGEFRVREGVSKPFTVRDHRGEAYKKHYPPTLDDEIWRLKKIAKDGASHKRLSKEGISCVKEFLRLFVTDPSLLRKMLACGNNIWEKITEHVSTCPLDNSEWYVYNAGESIVLLFNCIYELVGAILDGQNFQSIDKLDIFQKRMVEDFKRSVYKNLNCLAPLEDHSLIGQAILTSNLQNGLNHIPSSSQQNMNYSDEQGQVEFQSNSDHATISSPLAYTAQQDSPTTVSMPESFNGIQAFNSTLASSFLVGEPCSSIYPGDYDWGSNGSLDSLGMTDYLPPNNNYQFETPVWQGNELLASSSVQPVSPNIGFHITRRGNPRAIWCTIRAVLKWKTLVKRK

SEQ ID NO: 50 - protein, 549 aa, Solanum tuberosum
MVQKRNINQDNDENSEFPSRESKRQHPSNAFFSGLRSYSSPQELALRLEPSIRSWVREEIERTLQSTLRSSLNDDKTSQSRAIQLLFDDILPSSLFTGSNVSRDNRPIKVLLHDASSNQRITSGPLSSAKVDVVVLNGEFNPNDLEDWSEEEFSKKVIRAREGKRPLLTGDLIIQLRDGVGYLGDISFTDNSSWVKSRTFRLGLKLINRSGEFRVREGVSKPFTVRDHRGEAYKKHYPPTLDDEIWRLEKIAKDGASHKRLSNEGISCVKEFLRLFVTDPSLLRKMLACGNNIWEKITEHVSTCPLDNSEWYIYNAGESIVLLFNCIYELVGAILDGQNFQSIDKLDVFQKRMVEDFKRSVYKNLNCLAPLEDRSLIGQAALTSNLQNGLSHIPSSSQQNMNYSDEQGQVEFQSNSDHATISSPLAYTAQQDSPTAVSMPESFHGMQAFNSTLANSFLVGDPCSSIYPGDYDWGSNGSFDSLGMSDYLPPNNNYQFETPVWQGNGLLASSSVQPVSPNIGIHITRRGNPRAIWCTIRAVLKWKTLVKRK

SEQ ID NO: 51 - protein, 563 aa, Brassica napus
MKMRDRPFHGASGYSGLKAHKLTFKTAVKKVMKHMSNNQIMVHMENMFRKIVREELERLIQQHHLSSSWSQIERPRSETPSSRSRFKLRFINSPPPSIFTGAKIEPKNGFPLAIELVEAATNARVVSGPLSSSRVDFVPLNADFTEESWTVDLFKRYILKPREGKRPLLAGDVTLTLKNGVGVVSIAFTDNSIWSRCRKFRLGARLTGDGAVEARSEAFKCKDQRGESYKKHHPPYPGDEVWRLEKIGKDGASALRLAEREIYTVKDFRRCYAKDPNELYNILAGVGGGISKKIWESIVSHAMCCVLDETEYYIYDATGHDVSLVFNSVYEVTKVFIGGVLRNVDQLPSYQLDQLKREAYQNISRFRDSRTFADHPQRSLQCQQNPGFGPGFQQHMDFQGPSDTSMHAFFTGACSTSQPEMLMGFENSPSQTFHIDPTFIPTFRNSFRVNQHDMVHDELQSVVSRGYNKNNEDENGFAYHHHHEMPSNWSPGAAVWEQQEYNNLCVSVSGTEEAGYDVRIANIGGSPRARWCKVKAAFKLRQVWRHTSARKRGKACKKPCLLY

SEQ ID NO: 52 - protein, 576 aa, Gossypium hirsutum
MMLPTKRPAPDHGDDERNEVMVPEPKRRTNLKKNIMGVGGLSFNKIVLNLEPMLRIWVHEAVEAAIRSSIHPSSRPSLNRIEASRGRRLQLRFVDKPPSTIFTGSKVEAENGNPIRIILVDATSQAMVSSGSLSSIKVEIVVLNGEFGTDERQDWTENEFNASVLREREGRRPLVTGDLNITLVDGVGTVDNVIFTDNSSWIRCRKFRLGARIVQRSAGEVTIREATSDAFMVKDHRGELYKKHYPPLLHDEVWRLERIAKDGAFHKRLAYKNVHTVKDFLRLLVTDPIALRNILGGGISNRVWETIIEHALSCVPDDDEWYTYHGTAQRVELLLNSIYNVVKVTFDGQDYLPVENLTFSQKLLVEDAKRQAYKNVWNLTPFDRRAIMGPSMPFTDLLPEPVGTSNLLLQQHDFSGTRPDMPLGLNQSSTSFSYEVENPKPLQGIEPLNPMLRNSSFRMEGIFPYNADNSFSFFADDGFGTNQDNTQAQMLSLTSATPAWAQGSGFIFTPDYETSSISFLSSFPSCNVNDRSIGETREVCPKNRWLKVRAVIQWKSISRGAAKRRRQHWLQHGICT

SEQ ID NO: 53 - protein, 527 aa, Glycine max
MASKRTFDDEEDHQGNDDRDLGRLAKRRHGDPQEAEAMFIASLIASHLRPAIQKEIKEGLRSMFRGCACLCSPRSSINQQGGASTSGGRAMQLCFVNKLPIEFFTTFNITAEDGGPVQIELRYAGSQQRVVTEQVSNMKVQICVLDGDFGKDGNEDWSADEFNAQIVKPREGKGQLLKGETVIKLEKGFACINNKIEFTDNSIGTRNKKFRLGVKFLQSTSVSVSVREGRSEAFRVKDKRGEPYKKRDRPSLNDEVWCLKNIRRNGDLHKDLLKNKIKTVKDLLRLNTIGSLREKFGKVKKWDEIIEHAEKCAVDDDGFYMYRYDATVSLVLNCIYKVEAIFYGQHYRSLQSLNLEEQRLVERVKQEAYQNLQNLVPIPTPPPHDIVKTLTGTQYGSPDQGQQGQSELWISTTSTSIVHEAPLNLSGGSQVVPVETYNIDDGTGWDIGQFESYILPNQLLEPVNPCSFPYGDGATSSNHSFFLNPAVYTSSKGKSKTVWQKTRNILKWVIPHVTKWKVFRPQILQHY

SEQ ID NO: 54 - protein, 574 aa, Medicago truncatula
MASKRTSHTSDSCDDNVTSTSTKRIKQHHIHGNLEEEQQKEIQHLLFPTISASALALRLEPLLRKLIQEAIHLHLNPSCGLTLMSNTFGRRRLQFCFMNKLPNRIFTKSTIKAEGDEPLQIELRDVENQQRVVMEEGSSMKIQLCVLYGDFEKEDWTAEEFNTQIAPPREGKEELLKGNEFITLRNGVADIDKEIEFTDISKGRNGQFRLGVKIVQSNSIGVCIREGRSEPFKVLDVRGKNYEKHDRPSLNDEVWRLKGIRKNGPLDKLLASDGIHTVKDFLRLYITNEASLREKIGKIARNSWNTIVAHAKDCDVDNDDERYIYYSTEQPISFLVFNAIYKVVEVTFHNEQNARSIQSLNQQEKRLVERVKQHAYKYFNDWNPLPIDTTTLGLEETLTGVQNAQYDGQDQALQQSDFLVCQQGQKEIGQSYVQPCISTSYVNEGMDNYQIYVDPMPDIREIPQNNHVEGEMYIDGDGYGSHFPVVEGRYSMENLMNDYPIYTTCEPENYNLYGFSDVAECSTHVNFLDSSMDISSSDKSKAVWCKVGIAIKWVISIRRVAAAAKRNANLFYFN

SEQ ID NO: 55 - DNA, 2702 bp, Arabidopsis thaliana
  source 1..2702: mol_type = other DNA
aaagtcatgatgggaacaaggaatggaactcaaacattctaatactcaacaaacgcaattatattattaccatgactcatctttcaagttccatcaaaaagattcgtggaaaataatagacttacgtttcaaatccatgtttctttctttataacaaaaaaaatggatgtttcttgacgcgtgtcgagagtactcaccattactctgacttcagtgagtttggtcaagtggtctttttttttctcatgtcaccaaaggtccaaaccctagaaattagttcgaactttccatagaagaactgaataaatggtccaaaattgttttaaaaaggacctaagccattagttcattgaattcgagttaatgggtgaagatttttatgataacgaaagtcggagtaattatgcttttggtccgatagttttctaatttgttttctttccattttttttttttcaaatactacatactatataagatagtggtttgtgttaatgtcatcgatgtgttaccatccgcattatattaattatttatcccaacataaagtcagaatctgtaatttctttgttataaaatacagtaaatggttccgtttaagctgttagatgatttttgagtaaaaactaatgtaaaaaaaacaaaaaaaaaacaatgtagttcataatacatgcatgttttaaagaagtttcttgtttactatcaacttgaatagtatttcacgaagtcaaaattgttcattccgacttttctatgtggagaaaaaaaattctatcattgtgcacaatttaacagaatgtaatttcttgtaaaagaagaggaaacaattcgctgttagtaaatgtgaagtatagaagtctaaaatgagatacctcaactagcttgaattaagaaaaaaaacaaaaactctatcgacatgaaaaaggtcgcaaatatttatcatttatcaatgccaaaggagtatttggttcacaaaatactgaatcatttatatagatatataattagctctaaattctactataacttgcaaaataagtatactgactcaattatatagcgtttaaaaatagacgatttgtatgatgaggtccatatatatggagatgtgcatgcaactatcgacattttcacacgttgatatcgtctttctccaatggagacttgaatttgtgtaaactatgaatactcgtctctctaagaccttttttcttcaaccatgccaactatttaggtaagattttactgtctttgattgatattaaatacttagccgtggcgttatcaatgaatgataataaaaatgcggataaaagccaaaggtgttggaaataaatccaagaatgaagacgtagatgtcgatgggtattttaagaacttgaatttgtcacgactcacacgttaaaatatattatccgaattgtttagtctaaagacacacatatattgaaaaagaaaaggtaaatgaagctcattggtgcctaaatgtgaaatgaagccgaaatgtgttaggtgaacacatttaaatatacaaaaagaaatataatagaaacaaaactaattaacaaagtcgcaatttgtattgtataaaatatctttccgtctcccgtcatatttgaaaaaaaaaaaattacaaatctgttaattttaaaactttctagaaaaacacaagtatataattttctcttttcgtgcgtgtttgttttaaaataacattgttttgattggcgactcaacatattttagcatttacatatttctgcatatattaaatgatttataaactcaactatagattaaaatataatttgacatctaataattttaacaataatataaaatatgagatttataaattacgaatataaatattcaagggagagaaaaagtagaacataattcaaaagataagactttttagacttttttaacaatatttttgatggataaaaattattcaaaagagaagaaagtaagaagaaaagatgtttctgagaatttctagaaacagcatccgtttttataatttaattttcttacaaaggtaggaccaacatttgtgatctataaatcttcctactacgttatatagagacccttcgacataacacttaactcgtttatatatttgttttacttgttttgcacatacacacaaaaataaaaaagactttatatttatttactttttaatcacacggattagctccggcgaagtatggtcgtcgtcttcatcttcttcctccatcatcagatttttccttaaatggaagaaaccaaacgaaactccgatcttctccgttctcgtgttttcctctctggcttttattgctgggattgggaatttctcaccgctctcttgctttttagttgctgattctttttccttcgactttctatttccaatctttcttcttctctttgtgtattagattatttttagttttatttttctgtggtaaaataaaaaaagttcgccggagatgacggctgtgacggcggcgcaaagatcagttccggcgccgtttttaagcaaaacgtatcagctagttgatgatcatagcacagacgacgtcgtttcatggaacgaagaaggaacagcttttgtcgtgtggaaaacagcagagtttgctaaagatcttcttcctcaatacttcaagcataataatttctcaagcttcattcgtcagctcaacacttac

SEQ ID NO: 56 - DNA, 702 bp, Arabidopsis thaliana
  source 1..702: mol_type = other DNA
tctagaaacagcatccgtttttataatttaattttcttacaaaggtaggaccaacatttgtgatctataaatcttcctactacgttatatagagacccttcgacataacacttaactcgtttatatatttgttttacttgttttgcacatacacacaaaaataaaaaagactttatatttatttactttttaatcacacggattagctccggcgaagtatggtcgtcgtcttcatcttcttcctccatcatcagatttttccttaaatggaagaaaccaaacgaaactccgatcttctccgttctcgtgttttcctctctggcttttattgctgggattgggaatttctcaccgctctcttgctttttagttgctgattctttttccttcgactttctatttccaatctttcttcttctctttgtgtattagattatttttagttttatttttctgtggtaaaataaaaaaagttcgccggagatgacggctgtgacggcggcgcaaagatcagttccggcgccgtttttaagcaaaacgtatcagctagttgatgatcatagcacagacgacgtcgtttcatggaacgaagaaggaacagcttttgtcgtgtggaaaacagcagagtttgctaaagatcttcttcctcaatacttcaagcataataatttctcaagcttcattcgtcagctcaacacttac

SEQ ID NO: 57 - DNA, 48 bp, Arabidopsis thaliana
  source 1..48: mol_type = other DNA
atggtcgtcgtcttcatcttcttcctccatcatcagatttttccttaa

SEQ ID NO: 58 - protein, 15 aa, Arabidopsis thaliana
MVVVFIFFLHHQIFP

SEQ ID NO: 59 - DNA, 111 bp, Arabidopsis thaliana
  source 1..111: mol_type = other DNA
atggaagaaaccaaacgaaactccgatcttctccgttctcgtgttttcctctctggcttttattgctgggattgggaatttctcaccgctctcttgctttttagttgctga

SEQ ID NO: 60 - protein, 36 aa, Arabidopsis thaliana
MEETKRNSDLLRSRVFLSGFYCWDWEFLTALLLFSC

SEQ ID NO: 61 - DNA, 477 bp, synthetic construct
  source 1..477: mol_type = other DNA
aacagcatccgtttttataatttaattttcttacaaaggtaggaccaacatttgtgatctataaatcttcctactacgttatatagagacccttcgacataacacttaactcgtttatatatttgttttacttgttttgcacatacacacaaaaataaaaaagactttatatttatttactttttaatcacacggattagctccggcgaagtatggtcgtcgtcttcatcttcttcctccatcatcagatttttccttaaatggaagaaaccaaacgaaactccgatcttctccgttctcgtgttttcctctctggcttttattgctgggattgggaatttctcaccgctctcttgctttttagttgctgattctttttccttcgactttctatttccaatctttcttcttctctttgtgtattagattatttttagttttatttttctgtggtaaaataaaaaaagttcgccggag

SEQ ID NO: 62 - DNA, 219 bp, Arabidopsis thaliana
  source 1..219: mol_type = other DNA
atgacggctgtgacggcggcgcaaagatcagttccggcgccgtttttaagcaaaacgtatcagctagttgatgatcatagcacagacgacgtcgtttcatggaacgaagaaggaacagcttttgtcgtgtggaaaacagcagagtttgctaaagatcttcttcctcaatacttcaagcataataatttctcaagcttcattcgtcagctcaacacttac

(19) United States
(12) Patent Application Publication
(10) Pub. No.: US 2025/0027102 A1
(43) Pub. Date: Jan. 23, 2025

(54) Compositions and Methods for Safeguarding Plant Immunity Response to Elevated Temperatures

(71) Applicant: Duke University, Durham, NC (US)

(72) Inventors: Sheng-Yang He, Durham, NC (US); Jonghum Kim, Pohang (KR); Christian Danve M. Castroverde, Kitchener, Ontario (CA)

(73) Assignee: Duke University, Durham, NC (US)

(21) Appl. No.: 18/757,189

(22) Filed: Jun. 27, 2024

Related U.S. Application Data
(60) Provisional application No. 63/523,390, filed on Jun. 27, 2023.

(51) Int. Cl. C12N 15/82; C07K 14/415; G01N 33/00

(52) U.S. Cl. CPC C12N 15/8273; C07K 14/415; C12N 15/8279; G01N 33/0098

(57) ABSTRACT

The present disclosure describes, in part, compositions and methods for safeguarding plant immunity to pathogens in response to environmental changes.

### CROSS-REFERENCES TO RELATED APPLICATIONS

The present application claims priority to U.S. Provisional Application No. 63/523,390 filed Jun. 27, 2023, the full disclosure of which is incorporated by reference in its entirety for all purposes.

### REFERENCE TO SEQUENCE LISTING

The contents of the electronic sequence listing (106707-1443686-DU7642US SeqList.xml; Size: 71.9 bytes; and Date of Creation: Jun. 25, 2024) is herein incorporated by reference in its entirety.

### BACKGROUND

Extreme weather conditions associated with environmental changes impact many aspects of plant and animal life, including immune responses to infectious diseases. Salicylic acid (SA) is central to plant immunity to pathogens and insects. However, its production and signaling are particularly vulnerable to suppression by short periods of elevated temperature (ET) that simulate heat waves above the normal growth thermal range, which have become frequent events. The molecular basis of heat wave-mediated suppression of SA production and signaling remain poorly understood, representing a significant concern for crop protection and natural ecosystem conservation in a warming climate.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

The present disclosure is based, in part, on the discovery by the inventors that CBP60g-associated machinery is a primary target of SA-dependent immune suppression by elevated temperatures (ET) in plants.

A first aspect of the present application is directed to a plant comprising a genome having inserted into a genomic site thereof a constitutive promoter operably linked to a coding sequence comprising a pathogen-responsive upstream open reading frame (uORF) element and a polynucleotide sequence encoding a CBP60g protein, wherein the plant maintains salicylic acid (SA)-mediated immunity to a pathogen when under an environmental stress.

A second aspect of the present application is directed to a progeny of the plant of the first aspect.

A third aspect of the present application is directed to a method of making a plant that maintains salicylic acid (SA)-mediated immunity to a pathogen under an environmental stress. The method comprises introducing at a genomic site in a genome of a plant of interest a nucleic acid comprising a constitutive promoter operably linked to a coding sequence comprising a pathogen-responsive upstream open reading frame (uORF) element and a polynucleotide sequence encoding a CBP60g protein.

A fourth aspect of the present application is directed to a method for screening a subject plant for increased immunity to a pathogen when under an environmental stress. The method comprises the steps of: incubating a first copy of the subject plant with the pathogen under normal environmental growing conditions for a defined time period, wherein the subject plant comprises a plant made according to the method of the third aspect; incubating a first copy of a control plant with the pathogen under the normal environmental growing conditions for the defined time period, wherein the control plants is the same species as the plant of interest of the third aspect and is susceptible to the pathogen under the environmental stress; incubating a second copy of the subject plant with the pathogen under conditions comprising the environmental stress for the defined time period; incubating a second copy of the control plant with the pathogen under conditions comprising the environmental stress for the defined time period; assessing the first and second copies of the subject plant and the first and second copies of the control plant for characteristics of exposure to the pathogen at the end of the defined time period; and indicating that the subject plant has (i) increased immunity to a pathogen when under an environmental stress if the second copy of the subject plant exhibits fewer and/or reduced characteristics of exposure to the pathogen after the defined time period as compared to the second copy of the control plant after the defined time period, or (ii) does not have increased immunity to a pathogen when under an environmental stress if the second copy of the subject plant exhibits similar characteristics of exposure to the pathogen after the defined time period as compared to the second copy of the control plant after the defined time period.

### BRIEF DESCRIPTION OF THE DRAWINGS

The accompanying Figures and Examples are provided by way of illustration and not by way of limitation. The foregoing aspects and other features of the disclosure are explained in the following description, taken in connection with the accompanying example figures (also “FIG.”) relating to one or more embodiments, in which:

FIGS. 1A-1I show elevated temperature vulnerability of CBP60g gene expression and the SA transcriptome. Leaves of 4- to 5-week-old Arabidopsis plants were syringe-infiltrated with mock (0.25 mM MgCl₂), Pst DC3000 [1.0×10⁶ Colony Forming Units (CFU) ml-1] suspension or BTH (SA analog) solution and then incubated at 23° C. and 28° C. RNA-Seq and RT-qPCR were performed at 24 h after treatment [i.e., 1-day post-inoculation (dpi)]. FIG. 1A is a schematic diagram of experimental flow. ICS/transcript (FIG. 1B) and SA (FIG. 1C) levels observed in mock- and Pst DC3000-infiltrated Col-0 plants at 1 dpi. SA levels of mock- and Pst DC3000-inoculated Col-0 (FIG. 1D) and 35S::ICS1 or nprI^{S11D/S15D} (FIG. 1E) plants at 1 dpi. FIG. 1F shows the endogenous CBP60g transcript level of samples in FIG. 1B at 1 dpi. FIG. 1G shows GUS reporter gene expression in mock-, Pst DC3000- and BTH-treated p CBP60g::GUS plants at 1 day after treatment. FIG. 1H illustrates Gene Ontology (GO) enrichment of Pst DC3000-induced genes that are differentially regulated at elevated temperature and their overlap with the SARD1/CBP60g ChIP-Seq dataset (Sun, T., 2015). FIG. 1I shows representative RNA-Seq reads after Pst DC3000 infection of defense-related CBP60g target genes for plants in FIG. 1H. The data in 1B-1G and 1I are the means±S.D. [n=3 (FIGS. 1C, 1G, and 1I) or 4 (FIGS. 1B, ID, and 1F)] biological replicates from one representative experiment analyzed with two-way ANOVA with Tukey's HSD for significance. Experiments were independently performed three times, except for (i) with two experiments.

FIGS. 2A-2G show the SA pathway is downregulated at elevated temperatures in different plant species examined. SA levels are shown in 4-week-old Col-0 plants at 24 h after treatment [i.e., 1 day post-inoculation (dpi)] with flg22 peptide treatment (FIG. 2A) or Pst DC3000 (avrRps4) inoculation [1.0×10⁸ Colony Forming Units (CFU) ml⁻¹] (FIG. 2B) at 23° C. and 28° C. Transcript levels are shown for BnaPR1 in leaves of 4-week-old rapeseed Westar plants infiltrated with mock (0.25 mM MgCl₂) or Pst DC3000 [1.0×10⁶ Colony Forming Units (CFU) mL-1] (FIG. 2C) and NtPR1 in leaves of 4-week-old tobacco plants infiltrated with mock (0.25 mM MgCl²) or Ps tabaci 11528 [1.0×10⁶ Colony Forming Units (CFU) ml⁻¹] (FIG. 2D) at 24 h post-inoculation (1 dpi) at 23° C. and 28° C. FIG. 2E shows BnaPR1 expression levels in leaves of 4-week-old rapeseed Westar plants 1 day after mock (0.1% DMSO) or 50 μM BTH treatment at 23° C. and 28° C. FIG. 2F shows SA marker gene (SIPR1b) expression levels in 4-week-old Castlemart tomato plants 1 day after mock (0.1% DMSO) or 100 μM BTH treatment at 23° C. and 32° C. FIG. 2G shows SA marker gene (OsPR1b) expression levels in 5-week-old rice plants 1 day after mock (0.1% DMSO) or 200 μM BTH treatment at 28° C. and 35° C. Results show the means±S.D. [n=3 (FIGS. 2C and 2E-2G) or 4 (FIGS. 2A, 2B, and 2D) biological replicates] from one representative experiment (of three independent experiments) analyzed with two-way ANOVA with Tukey's HSD for significance.

FIGS. 3A-3P show basal resistance to Pst DC3000 at control (23° C.) and elevated temperature (28-30° C.) in constitutively activated phyB and ELF3 thermosensor lines and in genetically activated SA biosynthetic and signaling mutants. Symptom expression at 3 day post-inoculation (dpi) (FIGS. 3A and 3D), in planta Pst DC3000 bacterial levels at 3 dpi (FIGS. 3B and 3E) and SA levels of mock (0.25 mM MgCl₂)- and Pst DC3000-inoculated leaves [1.0×10⁶ Colony Forming Units (CFU) ml-1] at 1 dpi (FIGS. 3C and 3F) of Ler (FIGS. 3A-3C), Col-0 (FIGS. 3D-3F), 35S::PHYBY^{276H} (FIGS. 3A-3C), and BdELF3-OE (FIGS. 3D-3F). Symptom expression at 3 dpi (FIGS. 3G and 3I) and in planta Pst DC3000 bacterial levels at 3 dpi (FIGS. 3H and 3J) of Col-0 (FIGS. 3G-3J), 35S::ICS1 (FIGS. 3G and 3H), and npr 1^{S11D/S15D} (FIGS. 3I and 3J). Symptom expression at 3 dpi (FIGS. 3K and 3N), in planta Pst DC3000 bacterial levels at 3 dpi (FIGS. 3L and 3O) and SA levels of mock- and Pst DC3000-inoculated leaves at 1 dpi (FIGS. 3M and 3P) of Col-0 (FIGS. 3K-3P), npr3/4 (FIGS. 3K-3M), and 35S::TGA1 (FIGS. 3N-3P). Results show the means±S.D. [n=4 (FIGS. 3C, 3E, 3J, 3L, 3M, and 3P) or n=3 (FIGS. 3H and 3O) biological replicates] from one representative experiment (of three independent experiments) analyzed with two-way ANOVA with Tukey's HSD for significance. Results show the means±S.D. [(FIG. 3B) n=4 biological replicates except 35S::PHYB^{Y276H} at 30° C. (n=3 biological replicates), (FIG. 3F) n=4 biological replicates except BdELF3 OE, Pst at 23° C. (n=3 biological replicates)] from one representative experiment (of three independent experiments) analyzed with two-way ANOVA with Tukey's HSD for significance.

FIGS. 4A-4M show the effect of elevated temperature on transcript levels, protein levels and promoter recruitment of SA pathway regulators. Endogenous EDS1 (FIG. 4A), PAD4 (FIG. 4B), WRKY75 (FIG. 4C), and BSMT1 (FIG. 4D) transcript levels are shown for the samples in FIG. 1B at 24 h after treatment (1 dpi). FIG. 4E shows CBP60g gene expression levels in Col-0 and npr1-6 plants at 24 h after Pst DC3000 inoculation [1.0×10⁶ Colony Forming Units (CFU) ml⁻¹] at 23° C. FIG. 4F shows ChIP-qPCR analysis of 35S::TGA1-4myc using the anti-myc antibody and primer sets indicated in FIG. 5F. Binding of TGA1-myc to CBP60g locus is not affected by temperature in mock (0.1% DMSO)- or BTH-treated samples (P-value-0.7903 and 0.9566, respectively). FIG. 4G shows immunoblot results of 35S::TGA1-myc used for ChIP-qPCR analyses in FIG. 4F. FIG. 4H shows NPR1 immunoblot of NPR1pro::NPR1-YFP plant cytosolic and nuclear protein fractions 24 h after BTH treatment at 23° C. and 28° C. Both NPR1 oligomers (high molecular weight) and monomers (low molecular weight) are indicated by arrowheads. Anti-UGPase immunoblot is shown as the cytoplasmic marker control. FIG. 4I shows the ChIP-qPCR results of NPR1pro::NPR1-YFP using anti-MED6 antibody and primer sets indicated in FIG. 5F. FIG. 4J shows immunoblot result of MED16pro::MED 16-3flag used for ChIP-qPCR analysis in FIG. 5E. FIG. 4k shows the immunoblot results of NPR1pro::NPR1-YFP using anti-MED6 antibody used for ChIP-qPCR analyses in FIG. 4I. FIG. 4L depicts the ChIP-qPCR results of 35S::CDK8-myc using anti-myc antibody and primer sets indicated. FIG. 4M shows immunoblot results of 35S::CDK8-myc using anti-myc antibody used for ChIP-qPCR analyses in FIG. 4L. For immunoblot (FIGS. 4G, 4J, 4K, and 4M), stained RuBisCO large subunits are shown as loading controls. Numbers in panels (FIGS. 4G, 4H, 4J, 4K, and 4M) indicate relative protein band signal intensities compared to the corresponding band denoted with a * symbol(s). For ChIP analyses, the TA3 transposon was used as the negative control target locus. Primers are listed in Table 2. Antibody information is included in Table 3. Result in (FIGS. 4A-4E) shows the means±S.D. [n=4 (FIGS. 4A-4D) or 3 (FIG. 4E) biological replicates] from one representative experiment [of three (FIGS. 4A-4D) or two (FIG. 4E) independent experiments] analyzed with two-way ANOVA with Tukey's HSD for significance. Results in (FIGS. 4G, 4H, 4J, 4K, and 4M) show one representative experiment [of two (FIGS. 4G and 4H) or three (FIGS. 4J, 4K, and 4M) independent experiments]. Results in (FIGS. 4F, 4I, AND 4L) are the average±S.D. [of three independent experiments (n=3 experiments)], analyzed with two-way ANOVA with Tukey's HSD for significance.

FIGS. 5A-5F show that elevated temperature represses CBP60g promoter activity. Four- to 5-week-old Col-0 and various transgenic plants were treated with mock (0.1% DMSO) or 100 μM BTH solution and then incubated at 23° C. and 28° C. ChIP-qPCR and confocal imaging were performed in plants 1 day after treatment. FIG. 5A shows ChIP-qPCR analyses of NPR1pro::NPR1-YFP using anti-GFP antibody and primer sets indicated. FIG. 5B shows confocal imaging of eGFP-GBPL3 in 35S::eGFP-GBPL3 infiltrated with mock (0.1% DMSO), 200 μM SA or 100 μM BTH solution at control or elevated temperature 1 day after treatment. ChIP-qPCR analyses of various plant genotypes [35S::eGFP-GBPL3 (FIG. 5C), NPR1pro::NPR1-YFP (FIG. 5D), and MED16pro::MED16-flag (FIG. 5E)] using antibodies and primer sets indicated are shown. FIG. 5F shows known regulators at the CBP60g locus. Primer positions (P1 for promoter region and P2 for coding region) are indicated in the model. For ChIP analyses, the TA3 transposon was used as the negative control target loci in FIGS. 5A, 5C, 5D, and 5E. BTH-treated Col-0 sample at 23° C. in FIGS. 5C and 5E was used as a negative control of immunoprecipitation. Results in FIGS. 5A and 5C-5E are the average±S.D. of three independent experiments (n=3 experiments), analyzed with two-way ANOVA with Tukey's HSD for significance. For confocal imaging in FIG. 5B, results show one representative experiment (of four independent experiments) analyzed with one-way ANOVA with Bartlett's test for significance.

FIGS. 6A-6H show the characterization of 35S::eGFP-GBPL3 and GBPL3 OX plants. FIG. 6A shows CBP60g gene expression levels in Col-0, gbpl3-3, and 35S::eGFP-GBPL3 plants at 24 h (1 day) after mock (water) or 200 μM salicylic acid spray at 23° C. FIG. 6B illustrates immunoblot results of 35S::eGFP-GBPL3 used for ChIP-qPCR analyses in FIG. 5C. Stained RuBisCO large subunits are shown as loading controls. Numbers in the panel indicate relative protein band signal intensities compared to the corresponding band denoted with a * symbol. FIG. 6C shows subcellular fractionation of Arabidopsis Col-0 leaf cells treated with mock (0.1% DMSO) or BTH for 24 h at control (23° C.) or elevated temperature (28° C.). Actin and Histone H3 protein were used as markers of cytoplasmic and nuclear fractions, respectively. FIG. 6D shows in planta Pst DC3000 [1.0×10⁶ Colony Forming Units (CFU) ml⁻¹] bacterial levels in Col-0, GBPL3 OX #16 and GBPL3 OX #20 plants at 3 dpi. FIG. 6E shows CBP60g gene expression levels of Col-0 and GBPL3 OX #20 plants at 24 h after mock (0.1% DMSO) or 100 μM BTH spray at 23° C. or 28° C. FIG. 6F shows time lapse confocal microscopy of Arabidopsis mesophyll cell expressing eGFP-GBPL3 after transfer to 28° C. from 23° C. or to 23° C. from 28° C. Scale bar, 10 μm. FIG. 6G is a prediction of intrinsically disordered region in AtMED15 (Threshold score: 0.5). FIG. 6H shows confocal microscopy of Nicotiana tabacum mesophyll cells transiently expressing eGFP-GBPL3 and mRFP-MED15 at 23° C. and 28° C. Six to seven weeks old N. tabacum leaves were infiltrated with Agrobacterium harboring 35S::eGFP-GBPL3 or 35S::mRFP-MED15-flag. After incubation for 3 days at control temperature, the plants were treated with 100 μM BTH solution and shifted to 23° C. or 28° C. After 1 day, mesophyll cells were visualized by confocal microscopy. Scale bar, 10 μm. Results in (a, d, e) show the means±S.D. [(FIG. 6A) n=4, (FIG. 6D) n=4 except GBPL3 OX 16 at 23° C. (n=3 biological replicates), or (FIG. 6E) n=3 biological replicates] from one representative experiment (of two independent experiments), analyzed with two-way ANOVA with Tukey's HSD for significance. Results in FIG. 6B, left panel of FIG. 6C, show one representative experiment (of three independent experiments). Result in right panel of FIG. 6C shows the means±S.D. (of three independent experiments) analyzed with one-way ANOVA with Bartlett's test for significance. Results in FIGS. 6F and 6H show one representative experiment (of two independent experiments).

FIGS. 7A-7F show restoration of SA accumulation and immunity at elevated temperature in 35S::CBP60g plants. Wild-type Col-0 and 35S::CBP60g plants were syringe-infiltrated with mock (0.25 mM MgCl₂) or Pst DC3000 solution [1.0×10⁶ Colony Forming Units (CFU) mL-1] and then incubated at 23° C. and 28° C. in FIG. 7A, SA levels of mock- and Pst DC3000-inoculated plants at 24 h (1 dpi). FIG. 7b shows symptom expression of Pst DC3000-inoculated plants at 3 dpi. FIG. 7C shows in planta Pst DC3000 levels at 3 dpi. In planta Pst DC3000 (avrPphB; FIG. 7D) or (avrRps4; FIG. 7E) levels are shown at 3 dpi. FIG. 7F shows a heat map of RNA-Seq reads for genes that are downregulated in Col-0 but their expression fully/partially restored in 35S::CBP60g at 28° C. Results in FIGS. 7A, 7C, and 7E show the means±S.D. (n=4 biological replicates) for one representative experiment (of three independent experiments) analyzed with two-way ANOVA with Tukey's HSD for significance. Results in FIG. 7D show the means±S.D. [n=4 biological replicates except 35S::CBP60g at 23° C. (n=3 biological replicates)] for one representative experiment (of three independent experiments) analyzed with two-way ANOVA with Tukey's HSD for significance.

FIGS. 8A-8L show characterization of 35S::CBP60g 16 and cbp60g-1 plants. FIG. 8A shows CBP60g transcript levels in 4-week old 35S::CBP60g at 23° C. or 28° C. 1 day after mock (0.25 mM MgCl₂) treatment or Pst DC3000 infection [1.0×10⁶ Colony Forming Units (CFU) ml-1]. SA levels at 1 dpi (FIG. 8B), symptom expression at 3 dpi (FIG. 8C) and in planta Pst DC3000 [1.0×10⁶ Colony Forming Units (CFU) ml-1] bacterial levels at 3 dpi (FIG. 8D) of Col-0 and 35S::CBP60g 16 plants are depicted. Bacterial levels in Col-0 and cbp60g-1 plants inoculated with Pst DC3000 (FIG. 8E), Pst DC3000 (avrPphB) (FIG. 8F), and Pst DC3000 (avrRps4) (FIG. 8G) are shown at 3 dpi. FIG. 8H shows ICS1 gene expression levels in Pst DC3000 ΔhrcC-infected Col-0 and 35S:CBP60g plants (1.0×10⁸ Colony Forming Units (CFU) ml-1) at 12- and 24-hour post-inoculation (hpi). FIG. 8I shows SA levels in Pst DC3000 ΔhrcC-infected Col-0 and 35S:CBP60g plants (1.0×10⁸ Colony Forming Units (CFU) ml-1) at 24 h post-inoculation. In planta Pst DC3000 (avrPphB) (FIG. 8J), and (avrRps4) (FIG. 8K) bacterial levels of Col-0 and 35S::CBP60g 16 plants are shown at 3 dpi. FIG. 8L shows ICS1, EDS1 and PAD4 gene expression levels of Col-0 and 35S::CBP60g plants 1 day after mock (0.25 mM MgCl₂)- and Pst DC3000-infiltration [1.0×10⁶ Colony Forming Units (CFU) ml-1]. Results show the means±S.D. [n=3 (FIGS. 8A, 8F, 8G, and 8H) or 4 (FIGS. 8B, 8D, and 8I) biological replicates] from one representative experiment [of two (FIGS. 8A, 8H, and 8I) or three (FIGS. 8B, 8D, 8F, and 8G) independent experiments] analyzed with two-way ANOVA with Tukey's HSD for significance. Results in FIG. 8E show the means #S.D. [n=4 biological replicates except Col-0 at 23° C. (n=3 biological replicates)] from one representative experiments (of three independent experiments) analyzed with two-way ANOVA with Tukey's HSD for significance. Results in FIG. 8J show the means±S.D. [n=4 (Col-0) or 3 (35S::CBP60g 16) biological replicates] from one representative experiments (of three independent experiments) analyzed with two-way ANOVA with Tukey's HSD for significance. Results in FIG. 8K show the means±S.D. [n=4 biological replicates except 35S:CBP60g 16 at 23° C. (n=3 biological replicates)] from one representative experiments (of three independent experiments) analyzed with two-way ANOVA with Tukey's HSD for significance.

FIGS. 9A-9E depict the characterization of 35S::SARD1 plants. FIG. 9A shows SA levels at 24 h, FIG. 9B shows symptom expression at day 3, and FIG. 9C shows in planta bacterial levels at day 3 post-inoculation with mock (0.25 mM MgCl₂) or Pst DC3000 solution [1.0×10⁶ Colony Forming Units (CFU) ml-1]. FIG. 9D shows SARD1 gene expression levels in 4-week-old plants of Col-0 and 35S::SARD1. FIG. 9E shows the appearance of 4.5-week-old Col-0 and 35S::SARD1 plants (lines b1 and b1) grown at 23° C. were infiltrated with 1×10⁶ CFU ml⁻¹ Pst DC3000 and further incubated at 28° C. for 3 days. Results in FIG. 9A show the means±S.D. [n-6 (Col-0, 23° C. mock and Col-0, 23° C. Pst), 7 (Col-0, 28° C. mock and Col-0, 28° C. Pst), 8 (all 35S::SARD1 b1 line data), 7 (35S::SARD1 b2 line, 23° C. mock), 8 (35S::SARD1 b2 line, 23° C. Pst), 8 (35S::SARD1 b2 line, 28° C. mock), or 7 (35S::SARD1 b2 line, 28° C. Pst) biological replicates from two independent experiments] analyzed with two-way ANOVA with Tukey's HSD for significance. Results in FIG. 9C show the means±S.D. [n=3 (Col-0 at 23° C.), 4 (Col-0 at 28° C.), 3 (35S::SARD1 b1 at 23° C.), 4 (35S::SARD1 b1 at 28° C.), or 3 (35S::SARD1 b2 at 23° C. and 28° C.) biological replicates] from one representative experiments (of four independent experiments) analyzed with two-way ANOVA with Tukey's HSD for significance. Results in FIG. 9D show the means±S.D. (n=3 biological replicates) from one representative experiments (of two independent experiments) analyzed with two-way ANOVA with Tukey's HSD for significance. Result in FIG. 9E shows one representative experiment of four independent experiments.

FIGS. 10A-10C show BnaICS and BnaPR1 transcript levels in transgenic rapeseed plants expressing AtCBP60g-myc. FIG. 10A is an exemplary schematic diagram of experimental flow using Agrobacterium-mediated transient expression system. FIG. 10B shows transcript levels of BnaICS1 and myc-tagged transgenes (mRIP-myc or AtCBP60g-myc) in mock (0.25 mM MgCl₂)- or Pst DC3000-infiltrated [1.0×10⁵ Colony Forming Units (CFU) ml-1] rapeseed leaves at 1 dpi. Leaves were preinfiltrated with Agrobacterium suspension 3 days before mock or Pst DC3000 treatment. Results in FIG. 10B are the means±S.D. (n=4 biological replicates from two independent experiments). Statistical analysis was performed using two-way ANOVA with Tukey's HSD. The experiment was repeated four times with similar results. FIG. 10C shows transcript levels of BnaICS1, BnaPR1 and AtCBP60g-myc in mock- or Pst DC3000-infiltrated [1.0×10⁵ Colony Forming Units (CFU) ml-1] wild-type and two independent 35S::A1CBP60g-myc transgenic rapeseed leaves. AtCBP60g transcript level in each leaf sample was quantified (bottom row). No ArCBP60g transcript was detected in Westar samples as control, whereas AtCBP60g transcript was detected in each 35S::AtCBP60g-myc sample. Data in FIG. 10C are the means S.E.M. (n=4 biological replicates). The experiment was repeated twice. Statistical analysis was performed using two-way ANOVA with Tukey's HSD. n.a.=not applicable.

FIGS. 11A and 11B show Pst DC3000 bacterial population levels in Arabidopsis Col-0 and the ics1 mutant. In planta Pst [1.0×10⁶ Colony Forming Units (CFU) ml-1] bacterial levels in Col-0 and ics1 (i.e., sid2-2) plants at 23° C. and 30° C. at 1 (FIG. 11A) and 3 (FIG. 11B) dpi. Data are the means±S.D. (n=4 biological replicates). The experiment was repeated three times. Statistical analysis was performed using two-way ANOVA with Tukey's HSD.

FIGS. 12A-12E show SA accumulation and basal immunity to Pst DC3000 at elevated temperature in plants altered in positive and negative SA regulators. a-e, SA levels at 1 dpi (left panels), symptom expression at 3 dpi (middle panels) and in planta Pst DC3000 bacterial levels at 3 dpi (right panels) of Col-0 (FIGS. 12A-12E) and 35S::EDS1 (FIG. 12A), 35S::PAD4 (FIG. 12B), 35S::WRKY75 (FIG. 12C), bsmt1 (FIG. 12D) and camta2/3 plants (FIG. 12E) [1.0×10⁶ Colony Forming Units (CFU) ml⁻¹]. Results show the means #S.D. [n=4 (FIGS. 12A, 12B, and 12D) biological replicates] from one representative experiment (of three independent experiments) analyzed with two-way ANOVA with Tukey's HSD for significance. Results in FIG. 12C show the means±S.D [left panel: n=4 biological replicates except 35S::WRKY75, Pst at 23° C. (n=3 biological replicates); right panel: n=3 biological replicates except 35S::WRKY75 at 23° C. (n=4 biological replicates)] from one representative experiment (of three independent experiments) analyzed with two-way ANOVA with Tukey's HSD for significance. Results in FIG. 12E show the means±S.D. [top panel: n=4 biological replicates except Col-0, Pst at 23° C. (n=3 biological replicates); bottom panel: n=4 biological replicates] from one representative experiment (of three independent experiments) analyzed with two-way ANOVA with Tukey's HSD for significance.

FIGS. 13A-13D show the characterization of 35S::ICS1, 35S::CBP60g and uORF-CBP60g plants. FIG. 13A shows the appearance of 6-week-old Col-0, 35S::ICS1, 35S::CBP60g and uORFs-CBP60g plants. FIG. 13B shows quantification of fresh weights of 6-week-old Col-0, 35S::ICS1, and 35S::CBP60g plants. FIG. 13C shows flowering time phenotypes of Col-0 and 35S::C′BP60g plants. FIG. 13D shows CBP60g transcript levels in 4-week old Col-0 and 35S::uORFs-CBP60g plants measured by RT-qPCR. Results in FIG. 13B show the means±S.D. [n=15 (Col-0, 35S::CBP60g), n=16 (35S::ICS1) biological replicates] from one representative experiment (of two independent experiments) analyzed with one-way ANOVA with Bartlett's test for significance. Results in FIG. 13C show the means±S.D. (n=4 biological replicates) from one representative experiment (of two independent experiments) with two-tailed Student's t-test. Results in FIG. 13D show the means±S.D. (n=4 biological replicates of two independent experiments) analyzed with one-way ANOVA with Bartlett's test for significance.

FIGS. 14A-14F show that optimized CBP60g expression leads to elevated temperature resilient SA defenses without growth/developmental tradeoffs. Col-0, 35S::CBP60g and 35S::uORFs_{TBF1}-CBP60g plants were syringe-infiltrated with mock (0.25 mM MgCl₂) or Pst DC3000 solution [1.0×10⁶ Colony Forming Units (CFU) ml-1] and then incubated at 23° C. and 28° C. In FIG. 14A, foliar disease symptoms were evaluated 3-day post-inoculation (dpi). FIG. 14b shows in planta Pst DC3000 bacterial levels of the samples in FIG. 14A at 3 dpi. FIG. 14C shows SA levels of samples in FIG. 14A at 1 dpi. FIG. 14D shows in planta Pst DC3000 (avrPphB) and Pst DC3000 (avrRps4) levels at 3 dpi. FIG. 14E shows 4-week-old plant fresh weights (left) and flowering times (right) for plant genotypes indicated. Without being bound by theory, FIG. 14F depicts a potential working model on how elevated temperature targets SA defense immune network through CBP60g expression. At normal temperature, CBP60g gene expression is induced after pathogen infection. CBP60g regulates various defense genes, including those involved in SA accumulation (e.g., ICS1, EDS1 and PAD4). At elevated temperature, recruitment of Mediator, GBPL3 and RNA Pol II to the CBP60g locus is impaired, leading to reduction of SA production and immunity at elevated temperature. Results in FIGS. 14B-14D show the means±S.D. (n=3 biological replicates) from one representative experiment (of three independent experiments) analyzed with two-way ANOVA with Tukey's HSD for significance. Results in FIG. 14E show the means±S.D. (n=12 biological replicates) from one representative experiment (of three independent experiments) one-way ANOVA with Bartlett's test for significance.

FIG. 15 illustrates an exemplary experimental design for testing plant lines for elevated temperature tolerance to pathogens.

FIGS. 16A and 16B illustrate the symptoms of clubroot disease (FIG. 15A) and bacterial speck disease (FIG. 15B).

FIG. 17 panels show comparison of economic traits between a transgenic 35S::CBP60g rapeseed line and a control cultivar Wester.

FIGS. 18A-18C show the symptoms of clubroot disease at elevated temperature (FIG. 18A), pathogen content of control and transgenic rapeseed plants at normal and elevated temperatures (FIG. 18B), and the percentage of disease classes in each of those groups (FIG. 18C).

FIG. 19 shows the resistance of transgenic rapeseed plants to bacterial speck pathogen at elevated temperatures.

FIG. 20A shows bacterial levels in Arabidopsis Col-0 (Control) and 35S::CBP60g plants 3 day after Pst DC3000 inoculation under normal and high-salinity (400 mM NaCl) conditions. FIG. 20B pictures leaves of Arabidopsis Col-0 (Control) and 35S::CBP60g plants 3 day after Pst DC3000 inoculation under normal and high-salinity conditions.

FIG. 21 shows a partial list of putative Arabidopsis CBP60g orthologs in a variety of crop plants.

### DETAILED DESCRIPTION

The following detailed description is presented to enable any person skilled in the art to make and use the aspects of the present application. For purposes of explanation, specific nomenclature is set forth to provide a thorough understanding of the present application. However, it will be apparent to one skilled in the art that these specific details are not required to practice the aspects of the present application. Descriptions of specific embodiments are provided only as representative examples. The present application is not intended to be limited to the embodiments shown but is to be accorded the widest possible scope consistent with the principles and features disclosed herein. With respect to the teachings in the present application, any issued patent, pending patent application, patent application publication, or non-patent literature described in this application is expressly incorporated by reference herein.

Unless defined otherwise, all technical and scientific terms used herein have the same meanings as commonly understood by one of skill in the art to which the disclosed method and compositions belong. The phrase “as used herein” and variants thereof refer to the entire disclosure of this application, as well as to the appended claims.

Articles “a” and “an” as used herein refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations, where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Extreme weather conditions associated with environmental changes impact many aspects of plant and animal life, including immune responses to infectious diseases. Salicylic acid (SA) is central to plant immunity to pathogens and insects. However, its production and signaling are particularly vulnerable to suppression by short periods of elevated temperature (ET) that simulate heat waves above the normal growth thermal range, which have become frequent events. The molecular basis of heat wave-mediated suppression of SA production and signaling remain poorly understood, representing a significant concern for crop protection and natural ecosystem conservation in a warming climate. Recent studies showed that Phytochrome B (phyB) and Early Flowering 3 (ELF3) proteins play a critical role in regulating plant growth and development at ET. The present application shows that phyB and ELF3 are not involved in heat wave-mediated suppression of SA production. Instead, formation of the GBPL3 biomolecular condensate, which regulates SA signaling, is negatively affected by heat waves. The altered GBPL3 condensate formation is linked to impaired recruitment of GBPL3 and SA-relevant Mediator subunits to the promoter of CBP60g, a master transcription factor gene of SA production. Unlike many SA signaling components examined, including the major SA receptor gene NPR1 and biosynthetic gene ICS1, optimized CBP60g expression was sufficient to overcome ET-mediated suppression of SA production and to boost not only basal immunity, but also effector-triggered immunity at ET without significant growth tradeoffs. CBP60g-family transcription factors are widely conserved across plant lineages. The successful identification of the CBP60g-associated transcription machinery as a primary target of SA-dependent immune suppression by ET promises a broadly applicable mechanism to safeguard a central plant defense hormone for a warming climate.

Warmer temperatures either within the optimal growth temperature range or short periods of elevated temperature (ET) that simulate heat waves above the optimal temperature range for plant growth have been shown to affect basal-level or pathogen-induced production of salicylic acid (SA), a central hormone that regulates plant defenses against pathogens, insects and abiotic stresses. The ET-sensitivity appears to be unique to the SA pathway, as other stress hormone pathways, such as jasmonate (JA) and abscisic acid (ABA), are upregulated at ET. However, the mechanism(s) underlying selective suppression of the SA pathway during heat waves above the optimal temperature range is unclear and even controversial, leaving a significant gap in our understanding of how a warming climate with frequent and extreme heat waves would impact the effectiveness of the plant immune system. This knowledge gap presents a major obstacle to developing climate-resilient crop plants in which SA-mediated plant defenses will likely fail to operate effectively, a significant concern for future agricultural productivity and ecosystem preservation.

The present application discloses a key plant transcription factor, CBP60g, as the primary rate-limiting step in SA production during heat waves, as exemplified using Arabidopsis thaliana plants as a model system. Specifically, it was found that GBPL3-mediated transcription of the CBP60g gene becomes defective during a heat wave. Optimized expression of CBP60g not only prevents plants from becoming hyper-susceptible to virulent pathogens, but also protected disease resistance (R) gene-mediated resistance, which is compromised at warm temperatures. The latter is especially important because R genes are widely used in crop fields to guard crops against pathogens and insects and their reduced effectiveness at warm temperatures could threaten the global crop protection system in the coming decades.

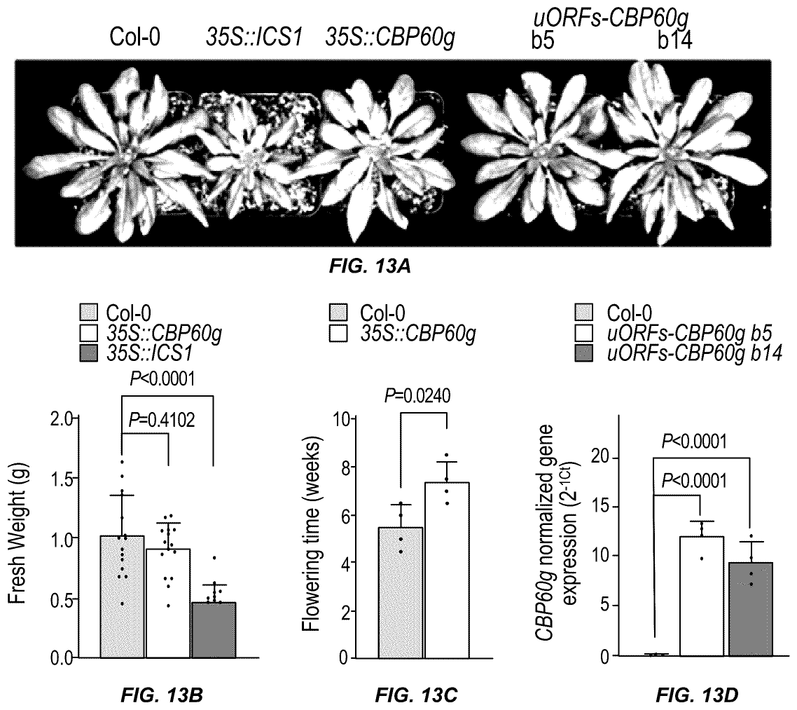

The SA defense pathway has been subjected to intense molecular genetic studies for more than three decades because of its central importance to plant immunity. Due to its ET-vulnerability, the effectiveness of SA-mediated immunity is becoming a significant concern for protection of crop production and natural ecosystems in a warming climate. As described in the present disclosure, the inventors identified CBP60g transcription as a major ET-vulnerable step in the plant immune system (FIG. 13F). it is disclosed here that ET negatively affects the formation of GBPL3 nuclear condensate and the recruitment of GBPL3 and SA-relevant Mediator subunits to the CBP60g promoter. It is notable that several recent studies have begun to implicate protein condensate formation/phase separation as a major regulatory process that controls temperature-regulated plant growth and flowering as well as hydration-dependent seed germination. Although the inventors found that ET-mediated suppression of SA defenses is independent of the ELF3 condensate-mediated thermosensing mechanism, ET affects GBPL3 condensate formation and the recruitment of GBPL3 and the Mediator subunits to the CBP60g promotor. Our results support an emerging general concept that biomolecular condensates serve as an important regulatory node for plant sensing and/or response to temperature and other environmental cues.

The identification of the CBP60g-associated transcription machinery as a rate-limiting step at ET prompted the inventors to redesign CBP60g expression to successfully rescue SA production and signaling at ET. CBP60g (along with GBPL3) is widely conserved across plant lineages. As such, the findings of the present inventors presented here could provide a broad framework for preserving SA-mediated immunity in diverse plants for a warming climate.

The inventors have surprisingly found that SA-mediated immunity in a plant can be restored in response to stress conditions, such as elevated temperature, by bypassing the GBPL3 pathway of condensate formation by placing the transcription of the CBP60g gene under the control of a constitutive promoter and the upstream open reading frame (uORF) of a pathogen-reactive gene such that SA-mediated immunity is preserved and/or restored in the plant.

Importantly, CBP60g and GBPL3 genes are found not only in Arabidopsis thaliana but are also widely conserved across diverse plant species. In addition to Arabidopsis, CBP60g protein orthologs have been widely identified in a variety of economically valuable plant species including, but not limited to, Brome (Brachpodium dystachion (SEQ ID NO: 44)), Sorghum (Sorghum bicolor (SEQ ID NO: 45)), Corn (Zea mays (SEQ ID NO: 46)), Barley (Hordeum vulgare (SEQ ID NO: 47)), Wheat (Triticum aestivum (SEQ ID NO: 48)), Tomato (Solanum lycopersicum (SEQ ID NO: 49)), Potato (Solanum tuberosum (SEQ ID NO: 50)), Rapeseed (Brassica napus (SEQ ID NO: 51)), Cotton (Gossypium hirsutum (SEQ ID NO: 52)), Soybean (Glycine max (SEQ ID NO: 53)), and Barrelclover (Medicago truncatula (SEQ ID NO: 54)). As such, the experiments described in the Examples of this disclosure provide a broad framework to prevent crop failures and natural ecosystem devastations due to compromised plant resistance against pathogens and insect pests in a warming climate.

### Plants

Provided in this disclosure are plants that maintain salicylic acid (SA)-mediated immunity to a pathogen when under an environmental stress as well as making such plants and screening plants for such capabilities. In some embodiments, the plants provided in this disclosure comprise a polynucleotide sequence encoding a CBP60g protein that is under the control of a constitute promoter and, further, comprises a pathogen-responsive transcriptional control element that permits translation of the CBP60g protein when the plant is exposed to a pathogen and has an immune response. In some embodiments, the pathogen-responsive transcriptional control element is a pathogen-responsive upstream open reading frame (uORF) element.

A first aspect of the present application is a plant comprising a genome having inserted into a genomic site thereof a constitutive promoter operably linked to a coding sequence comprising a pathogen-responsive upstream open reading frame (uORF) element and a polynucleotide sequence encoding a CBP60g protein, wherein the plant maintains salicylic acid (SA)-mediated immunity to a pathogen when under an environmental stress.

The term “plant” refers to any organism, which is capable of photosynthesis. Accordingly, encompassed within the scope of the term “plant” are all genera and species of higher and lower plants of the Plant Kingdom. Annual, perennial, monocotyledonous and dicotyledonous plants and gymnosperms are preferred. The class of plants of the first aspect of this disclosure and that can be used in the methods provided in this disclosure is generally as broad as the class of higher and lower plants amenable to transformation techniques, including angiosperms (monocotyledonous (monocot) and dicotyledonous (dicot) plants), gymnosperms, ferns, and multicellular algae. Accordingly, the plant is preferably, a monocot plant or a dicot plant. It includes plants of a variety of ploidy levels, including aneuploid, polyploid, diploid, haploid and hemizygous. Preferred are plants and plant materials of the following plant families: Amaranthaceae, Brassicaceae, Carophyllaceae, Chenopodiaceae, Compositae, Cucurbitaceae (such as Siraitiaaa grosverorii (monk fruit), Labiatae, Leguminosae, Papilionoideae, Liliaceae, Linaceae, Malvaceae (such as cotton, cacao, okra, roselle, and durian), Rosaceae, Saxifragaceae, Scrophulariaceae, Solanaceae, Tetragoniaceae. Annual, perennial, monocotyledonous and dicotyledonous plants are preferred plants. Said plants may include—but are not limited to—bryophytes such as, for example, Hepaticae (hepaticas) and Musci (mosses); pteridophytes such as ferns, horsetail and clubmosses; gymnosperms such as conifers, cycads, ginkgo and Gnetaeae; algae such as Chlorophyceae, Phaeophpyceae, Rhodophyceae, Myxophyceae, Xanthophyceae, Bacillariophyceae (diatoms) and Euglenophyceae. Plants for the purposes of the aspects of this disclosure may comprise the families of the Rosaceae such as rose, Ericaceae such as rhododendrons and azaleas, Euphorbiaceae such as poinsettias and croton, Caryophyllaceae such as pinks, Solanaceae such as petunias, Gesneriaceae such as African violet, Balsaminaceae such as touch-me-not, Orchidaceae such as orchids, Iridaceae such as gladioli, iris, freesia and crocus, Compositae such as marigold, Geraniaceae such as geraniums, Liliaceae such as Drachaena, Moraceae such as ficus, Asteraceae such as Stevie rebaudiana in particular, Araceae such as philodendron and many others. The plants according to the invention are furthermore selected in particular from among dicotyledonous crop plants such as, for example, from the families of the Leguminosae such as pea, alfalfa and soybean; the family of the Umbelliferae, particularly the genus Daucus (very particularly the species carota (carrot)) and Apium (very particularly the species graveolens var. dulce (celery)) and many others; the family of the Solanaceae, particularly the genus Lycopersicon, very particularly the species esculentum (tomato) and the genus Solanum, very particularly the species tuberosum (potato) and melongena (aubergine/eggplant), tobacco and many others; and the genus Capsicum, very particularly the species annum (pepper) and many others; the family of the Leguminosae, particularly the genus Glycine, very particularly the species max (soybean) and many others; and the family of the Cruciferae, particularly the genus Brassica, very particularly the species napus (oilseed rape), campestris (beet), oleracea (and all cultivars (cv) thereof, such as cabbage cv Tastie, cauliflower cv Snowball Y and broccoli cv Emperor); and the genus Arabidopsis, very particularly the species thaliana and many others; the family of the Compositae, particularly the genus Lactuca, very particularly the species sativa (lettuce) and many others. In some embodiments, the plant can be of the family Triticeae, such as barley, wheat, or rye. In some embodiments, the plant can be of the family Poaceae, such as corn, rice, oats, or sorghum. In some embodiments, the plant can be a fruit plant, a vegetable plant, a tree nut plant, or a cotton plant.

As used herein, a “disease” in the context of plants refers to any condition that affects the health or productivity of a plant, in particular resulting in an impairment of the normal state of a plant that interrupts or modifies its vital functions. Examples of plant diseases include, but are not limited to, clubroot, bacterial speck disease, soybean rust, wheat rust, rice blast, bacterial fire blight, bacterial wilt, and potato late blight. Plant diseases include abiotic and biotic plant diseases. Abiotic (or non-infectious) diseases are caused by conditions external to the plant, not living agents. They cannot spread from plant to plant. Examples of abiotic diseases include nutritional deficiencies, soil compaction, salt injury, ice, and sun scorch. Biotic (or infectious) diseases are caused by living organisms. They are called plant pathogens when they infect plants. Pathogens can spread from plant to plant and may infect all types of plant tissue including leaves, shoots, stems, crowns, roots, tubers, fruit, seeds and vascular tissues. Biotic diseases are caused by pathogens such as fungi, fungal-like organisms (oomycete), bacteria, phytoplasmas, viruses, viroids, nematodes, and parasitic higher plants are all plant pathogens. Plants that are under the stress of abiotic diseases can also be more susceptible to biotic diseases due to changes in the plant, such as gene expression, caused by an abiotic disease while acclimation to environmental conditions.

As used herein, the term “pathogen” refers to an agent or organism that causes disease in a plant. Examples of pathogens include, but are not limited to, fungi (such as Fusarium lycopersici, Colletotrichum species), oomycetes (such as Phytophthora infestans), bacteria (such as Pseudomonas syringae, Xanthomonas campestris, and Ralstonia solanacearum), phytoplasmas (such as Candidatus Phytoplasma), protists (such as Plasmodiophora brassicae and Plasmopara viticola), viruses (such as tobacco mosaic virus and tomato mosaic virus), and viroids (such as citrus exocortis viroid).

In some embodiments, the environmental stress is at least one of heat stress, drought, or salinity. In some further embodiments, the heat stress is an ambient temperature that is elevated over a maximum ambient temperature for the plant. The pathogen can be any of the pathogens described in this disclosure. In some embodiments, the pathogen is at least one of a fungus, a fungal-like organism, a bacterium, a phytoplasma, a protist, a virus, or a viroid. In some embodiments, the pathogen is a fungus. In certain embodiments, the fungus is Plasmodiophora brassicae. In certain embodiments, the pathogen is a bacterium. In certain embodiments, the bacterium is Pseudomonas syringae.

### CBP60g

In some embodiments, the polynucleotide sequence encoding a CBP60g protein is an exogenous polynucleotide sequence. For example, the polynucleotide sequence encoding a CBP60g protein may be a non-genomic sequence. In some embodiments, as discussed below, the polynucleotide sequence encoding a CBP60g protein may be introduced into the plant by transformation.

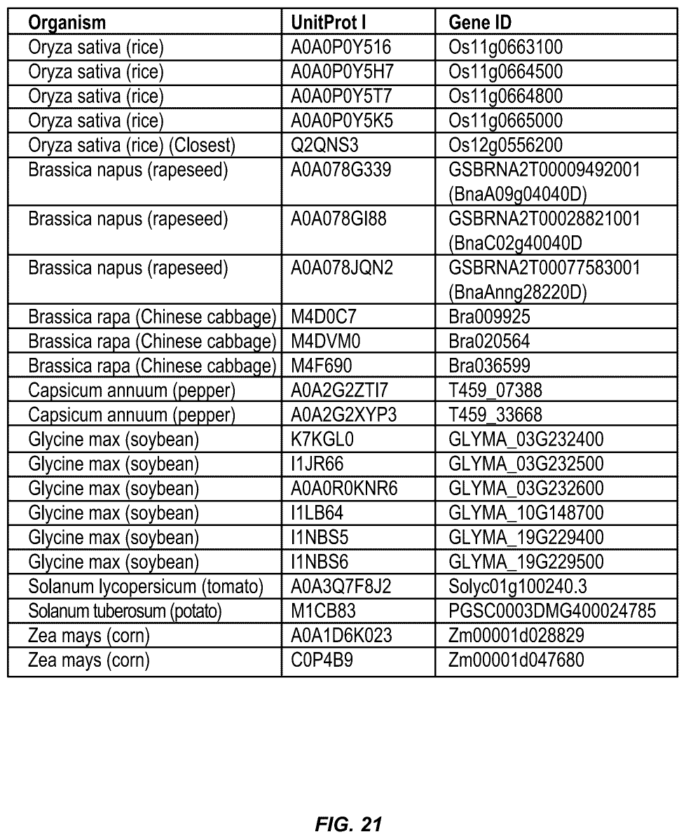

Various plant CBP60g gene orthologs are known and the proteins encoded thereby can be the CBP60g protein inserted into the plant provided by this disclosure. In some embodiments, the CBP60g protein is encoded by one of the putative Arabidopsis CBP60g ortholog genes in FIG. 21. In some embodiments, the CBP60g protein is Arabidopsis thaliana CBP60g protein, Brachpodium dystachion CBP60g protein, Sorghum bicolor CBP60g protein, Zea mays CBP60g protein, Hordeum vulgare CBP60g protein, Triticum aestivum CBP60g protein, Solanum lycopersicum CBP60g protein, Solanum tuberosum CBP60g protein, Brassica napus CBP60g protein, Gossypium hirsutum CBP60g protein, Glycine max CBP60g protein, Medicago truncatula CBP60g protein, or an ortholog or homolog thereof. In some embodiments, the CBP60g protein has at least 90% identity to any one of SEQ ID NOs: 43-53.

### Promoter

In some embodiments, the constitutive promoter is a heterologous promoter. The terms “heterologous promoter,” “promoter,” “promoter region,” or “promoter sequence” refer generally to transcriptional regulatory regions of a gene, which may be found at the 5′ or 3′ side of the insert site, or within the coding region of the heterologous coding sequence, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

As used herein, a polynucleotide is “operably connected” or “operably linked” when it is placed into a functional relationship with a second polynucleotide sequence. For instance, a promoter is operably linked to an insert site or heterologous coding sequence within the insert site if the promoter is connected to the coding sequence or insert site such that it may affect transcription of the coding sequence. In various embodiments, the polynucleotides may be operably linked to at least 1, at least 2, at least 3, at least 4, at least 5, or at least 10 promoters.

Promoters useful in the practice of the present invention include, but are not limited to, constitutive, inducible, temporally regulated, developmentally regulated, chemically regulated, tissue-preferred and tissue-specific promoters. Suitable promoters for expression in plants include, without limitation, the TBF1 promoter from any plant species including Arabidopsis, the 35S promoter of the cauliflower mosaic virus (CaMV), ubiquitin, 1 (UP cryptic constitutive promoter, the Rsyn7 promoter, pathogen-inducible promoters, the maize In2-2 promoter, the tobacco PR-1a promoter, glucocorticoid-inducible promoters, estrogen-inducible promoters and tetracycline-inducible and tetracycline-repressible promoters. Inducible promoters that can be used in the present invention include, but are not limited to, pathogen-inducible promoters PR-1a and CMPG1 of Arabidopsis, biotic stress-inducible promoter GST1 of potato, biotic stress-inducible promoter SGD24-STR246C of tobacco, and Zmap of maize, which responds to multiple stressors. Tissue-specific promoters usable in the present invention include, but are not limited to, the flower, seed and embryogenesis specific β-phaseolin promoter of Phaseolus vulgaris, ripening fruit specific EXPI promoter in banana, the bidirectional green tissue promoters GSSP1, GSSP3, GSSP5, GSSP6, GSSP7, of rice, mesocarp-specific MT3-A and leaf specific LCO1 promoters of oil palm, root specific SynR2 SynR1 promoter of tobacco, and the pCL promoter of potato, which provides gene regulation of the activity of acid vacuolar invertase in potato tubers at low temperature (Villao-Uzho, 2023). Other promoters include the T3, T7 and SP6 promoter sequences, which are often used for in vitro transcription of RNA. Synthetic plant promoters are also suitable for use in the present invention, including those that are tissue-specific, abiotic, hormonal. Light, biotic, chemical and constitutive (Dey, 2015). Those of skill in the art are familiar with a wide variety of additional promoters for use in various cell types. In some embodiments, the heterologous promoter includes a plant promoter. In some embodiments, the heterologous promoter includes a plant promoter inducible by a plant pathogen or chemical inducer. The heterologous promoter may be a seed-specific or fruit-specific promoter.

In some embodiments, the constitutive promoter to which the polynucleotide sequence encoding a CBP60g protein is operably linked is a plant promoter. In some embodiments, the constitutive promoter to which the polynucleotide sequence encoding a CBP60g protein is operably linked is a viral promoter. In some embodiments, the constitutive promoter is a Cauliflower Mosaic Virus (CaMV) 35S promoter.

In some embodiments, the constitutive promoter can be a modified CBP60g gene promoter comprising the sequence of the endogenous CBP60g gene promoter that has been modified to be constitutive.

uORF

uORFs are important Cis-regulatory elements in plants and are defined by a translation initiation codon in the 5′ untranslated region (5′ UTR) of a gene and an uORF-associated stop codon that can end before the main coding sequence (mORF) or overlap with it if the uORF is out of sequence with the mORF. uORFs can serve as rapid-response elements, allowing plants to immediately adopt protein production to altered environmental conditions at the level of transcription. For example, TL1-BINDING TRANSCRIPTION FACTOR 1 (TBF1) is an important transcription factor for the growth-to-defense switch upon immune induction in plants. TBF1 has been identified as a heat shock factor-like transcription factor that specifically binds to the TL1 cis element required for the induction of antimicrobial protein secretion in plants. The expression of TBF1 is tightly regulated at both the transcriptional and translational levels. Two uORFs encoding multiple aromatic amino acids are located upstream of the translation initiation codon of TBF1 and affect its translation. Translation of TBF1 is normally suppressed by two uORFs within the 5′ leader sequence. Through this unique regulatory mechanism, TBF1 can sense the metabolic changes upon pathogen invasion and trigger the specific transcriptional reprogramming through its target gene expression (Pajerowska-Mukhtar, 2012). SEQ ID NO: 55 includes the 2000 base pairs of upstream sequence (residues 1-2000), the 5′UTR (2001-2483), and the coding region (2484-2702) of A. thaliana TBF1 (NCBI 829853). BLAST analysis showed that uORF2TBF1, the major mRNA feature conferring the translational suppression, is conserved across plant species (>50% identity). See Xu, 2017, and Pajerowska-Mukhtar, 2012.

In some embodiments, the pathogen-responsive uORF element comprises at least one uORF sequence operably linked to the constitutive promoter. In some embodiments, the pathogen-responsive uORF element comprises at least one TBF1 gene uORF sequence. In some embodiments, the pathogen-responsive uORF element comprises one or two Arabidopsis thaliana TBF1 (AtTBF1) gene uORF sequences. In some embodiments, a first uORF has the sequence set forth in SEQ ID NO: 57 or a sequence having at least 50%, 60%, 70%, 80%, 85%, 86%, 87%, 88%, 89%, 90%, 91%, 92%, 93%, 94%, 95%, 96%, 97%, 98%, or 99% identity thereto. In some embodiments, the first uORF encodes a polypeptide having the sequence set forth in SEQ ID NO:58 or a sequence having at least 50%, 60%, 70%, 80%, 85%, 86%, 87%, 88%, 89%, 90%, 91%, 92%, 93%, 94%, 95%, 96%, 97%, 98%, or 99% identity thereto. In some embodiments, a second uORF has the sequence set forth in SEQ ID NO: 59 or a sequence having at least 50%, 60%, 70%, 80%, 85%, 86%, 87%, 88%, 89%, 90%, 91%, 92%, 93%, 94%, 95%, 96%, 97%, 98%, or 99% identity thereto. In some embodiments, the second uORF encodes a polypeptide having the sequence set forth in SEQ ID NO:60 or a sequence having at least 50%, 60%, 70%, 80%, 85%, 86%, 87%, 88%, 89%, 90%, 91%, 92%, 93%, 94%, 95%, 96%, 97%, 98%, or 99% identity thereto. In some embodiments, the pathogen-responsive uORF element comprises a sequence having at least 80%, 85%, 86%, 87%, 88%, 89%, 90%, 91%, 92%, 93%, 94%, 95%, 96%, 97%, 98%, or 99% identity to SEQ ID NO:61. In some embodiments, the pathogen-responsive uORF element is the sequence of SEQ ID NO:61.

In some embodiments, the plant provided in this disclosure comprises a genome having inserted into a genomic site thereof a constitutive promoter operably linked to a coding sequence comprising a pathogen-responsive upstream open reading frame (uORF) element, where in the genomic site is the location of the endogenous CBP60g gene promoter such that the endogenous CBP60g gene promoter is replaced by the constitutive promoter operably linked to a coding sequence comprising a pathogen-responsive uORF element. In such embodiments, the plant maintains salicylic acid (SA)-mediated immunity to a pathogen when under an environmental stress.

A second aspect of the present application is a progeny of the plant of the first aspect.

As exemplified herein, the present inventors made use of the pathogen responsiveness of the uOREs_{TBF1} (SEQ ID NO: 61) system by replacing the TBF1 gene (SEQ ID NO: 62) with cDNA (SEQ ID NO: 1) encoding the Arabidopsis CBP60g protein. The construct was put under the control of the CaMV 35S constitutive promoter. The 35S::HORESTBF1-CBP60g construct was introduced into the plant genome via Agrobacterium-mediated transformation. While t-DNA based Agrobacterium-mediated transformation is a form of random incorporation into the genome used in this exemplary system, the inventors have conceived that site-specific methods of incorporation, such as CRISPR/Cas9, can also be used. For example, using a site-specific method, a plant's endogenous CBP60g gene can be replaced by an expression construct responsive to the metabolic changes associated with pathogen infection. Alternatively, a plant's endogenous CBP6g0 gene can be placed under the control of the uORF derived from a pathogen-responsive gene.

In some embodiments, the constitutive promoter to which the polynucleotide sequence encoding a CBP60g protein is operably linked is a CaMV 35S promoter, and the pathogen-responsive uORF element comprises two AtTBF1 uORFs (uORF1: DNA—SEQ ID NO: 57, encoding amino acid-SEQ ID NO: 58; uORF2: DNA-SEQ ID NO: 59, encoding amino acid—SEQ ID NO: 60) operably linked to an AtTB1 promoter.

Regarding polynucleotide sequences, a “variant,” “mutant,” or “derivative” may be defined as a polynucleotide sequence having at least 50% sequence identity to the particular polynucleotide over a certain length of one of the polynucleotide sequences using blastn with the “BLAST 2 Sequences” tool available at the National Center for Biotechnology Information's website. Such a pair of polynucleotides may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Regarding polynucleotide sequences, the terms “percent identity” and “% identity” and “% sequence identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent sequence identity for a polynucleotide may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, MD, at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 2, at least 3, at least 10, at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Polynucleotides homologous to the polynucleotides described herein are also provided. Those of skill in the art also understand the degeneracy of the genetic code and that a variety of polynucleotides can encode the same polypeptide. In some embodiments, the polynucleotides (i.e., the uORF polynucleotides) may be codon-optimized for expression in a particular cell. While particular polynucleotide sequences which are found in plants are disclosed herein any polynucleotide sequences may be used which encode a desired form of the polypeptides described herein. Thus, non-naturally occurring sequences may be used. These may be desirable, for example, to enhance expression in heterologous expression systems of polypeptides or proteins. Computer programs for generating degenerate coding sequences are available and can be used for this purpose. Pencil, paper, the genetic code, and a human hand can also be used to generate degenerate coding sequences.

The polypeptides disclosed herein may include “variant” polypeptides, “mutants,” and “derivatives thereof.” As used herein the term “wild-type” is a term of the art understood by skilled persons and means the typical form of a polypeptide as it occurs in nature as distinguished from variant or mutant forms. As used herein, a “variant,” “mutant,” or “derivative” refers to a polypeptide molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. As discussed above with respect to polynucleotide sequences, a “variant,” “mutant,” or “derivative” of polypeptide sequences may be defined as having at least 50% sequence identity to the particular polypeptide over a certain length of one of the polypeptide sequences using blastn with the “BLAST 2 Sequences” tool available at the National Center for Biotechnology Information's website. Such a pair of polypeptides may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. Percent identity of polypeptide sequence variants, mutants, or derivatives can be measured as described above for polynucleotide sequences.

The polypeptides provided herein may be full-length polypeptides or may be fragments of the full-length polypeptide. As used herein, a “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 25, 30, 40, 50, 60, 70, 80, 90, 100, 150, 250, or 500 contiguous amino acid residues of a reference polypeptide. Fragments may be preferentially selected from certain regions of a molecule. The term “at least a fragment” encompasses the full-length polypeptide. A fragment of a uORF polypeptide may comprise or consist essentially of a contiguous portion of an amino acid sequence of the full-length uORF polypeptide. A fragment may include an N-terminal truncation, a C-terminal truncation, or both truncations relative to the full-length uORF polypeptide.

A “deletion” in a polypeptide refers to a change in the amino acid sequence resulting in the absence of one or more amino acid residues. A deletion may remove at least 1, 2, 3, 4, 5, 10, 20, 50, 100, 200, or more amino acids residues. A deletion may include an internal deletion and/or a terminal deletion (e.g., an N-terminal truncation, a C-terminal truncation or both of a reference polypeptide).

“Insertions” and “additions” in a polypeptide refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, 200, or more amino acid residues. A variant polypeptide may have N-terminal insertions, C-terminal insertions, internal insertions, or any combination of N-terminal insertions, C-terminal insertions, and internal insertions.

The amino acid sequences of the polypeptide variants, mutants, or derivatives as contemplated herein may include conservative amino acid substitutions relative to a reference amino acid sequence. For example, a variant, mutant, or derivative polypeptide may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

### Methods

Another aspect of the present application is a method of making a plant that maintains salicylic acid (SA)-mediated immunity to a pathogen under an environmental stress. The method comprises introducing at a genomic site in a genome of a plant of interest a nucleic acid comprising a constitutive promoter operably linked to a coding sequence comprising a pathogen-responsive upstream open reading frame (uORF) element and a polynucleotide sequence encoding a CBP60g protein. The features of the plant produced according to this method are those described above with respect to the plant of the first aspect of this disclosure.

In some embodiments, the step of introducing nucleic acid into the plant of interest comprises random incorporation of a construct into the genome of plant. In some embodiments, the random incorporation comprises t-DNA based Agrobacterium-mediated transfection.

In other embodiments, the genomic site is a location of an endogenous CBP60g gene in the genome of the plant of interest, and wherein the step of introducing the nucleic acid into the plant of interest results in replacement of all or a portion of the endogenous CBP60g gene in the genome of the plant of interest with the nucleic acid sequence such that the endogenous CBP60g gene is not expressed in the plant of interest.

As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies.

Optionally, in other embodiments, the DNA constructs may further include a uORF polynucleotide encoding any one of the uORF polypeptides as provided herein, or a variant thereof. Alternatively, the DNA constructs may include a heterologous promoter operably connected to a DNA polynucleotide encoding a RNA transcript including a 5′ regulatory sequence located 5′ to an insert site, wherein the 5′ regulatory sequence includes an uORF polynucleotide encoding any one of the uORF polypeptides as provided herein or a variant thereof.

As used herein, the terms “polynucleotide,” “polynucleotide sequence,” “nucleic acid,” and “nucleic acid sequence” refer to a nucleotide, oligonucleotide, polynucleotide (which terms may be used interchangeably), or any fragment thereof. These phrases also refer to DNA or RNA of natural or synthetic origin (which may be single-stranded or double-stranded and may represent the sense or the antisense strand).

The constructs provided herein may be prepared by methods available to those of skill in the art. Notably each of the constructs claimed are recombinant molecules and as such do not occur in nature. Generally, the nomenclature used herein, and the laboratory procedures utilized in the present invention include molecular, biochemical, and recombinant DNA techniques that are well known and commonly employed in the art. Standard techniques available to those skilled in the art may be used for cloning, DNA and RNA isolation, amplification and purification. Such techniques are thoroughly explained in the literature.

The DNA constructs of the present invention may include a heterologous promoter operably connected to a DNA polynucleotide encoding a RNA transcript including a 5′ regulatory sequence located 5′ to an insert site. Heterologous as used herein simply indicates that the promoter, 5′ regulatory sequence and the insert site or the coding sequence inserted in the insert site are not all natively found together.

An “insert site” is a polynucleotide sequence that allows the incorporation of another polynucleotide of interest. Exemplary insert sites may include, without limitation, polynucleotides including sequences recognized by one or more restriction enzymes (i.e., multicloning site (MCS)), polynucleotides including sequences recognized by site-specific recombination systems such as the λ phage recombination system (i.e., Gateway Cloning technology), the FLP/FRT system, and the Cre/lox system or polynucleotides including sequences that may be targeted by the CRISPR/Cas system. The insert site may include a heterologous coding sequence encoding a heterologous polypeptide.

A “5′ regulatory sequence” is a polynucleotide sequence that when expressed in a cell may, when DNA, be transcribed and may or may not, when RNA, be translated. For example, a 5′ regulatory sequence may include polynucleotide sequences that are not translated but control, for example, the translation of a downstream open reading frame (i.e., heterologous coding sequence). A 5′ regulatory sequence may also include an open reading frame (i.e., uORF) that is translated and may control the translation of a downstream open reading frame (i.e., heterologous coding sequence). In accordance with the present disclosure, the 5′ regulatory sequence is located 5′ to an insert site.

The DNA constructs of the present invention may also include a heterologous promoter operably connected to a DNA polynucleotide encoding a RNA transcript including a 5′ regulatory sequence located 5′ to an insert site, wherein the 5′ regulatory sequence includes a uORF polynucleotide encoding any one of the uORF polypeptides of a sequence as provided herein or a variant thereof.

The constructs of the present invention may include an insert site including a heterologous coding sequence encoding a heterologous polypeptide, in particular, the CBP60g protein. In some embodiments, the expression of the constructs of the present invention in a cell produces a transcript including the heterologous coding sequence and a 5′ regulatory sequence. A “heterologous coding sequence” is a region of a construct that is an identifiable segment (or segments) that is not found in association with the larger construct in nature. When the heterologous coding region encodes a gene or a portion of a gene, the gene may be flanked by DNA that does not flank the genetic DNA in the genome of the source organism. In another example, a heterologous coding region is a construct where the coding sequence itself is not found in nature.

A “heterologous polypeptide,” “polypeptide,” “protein,” or “peptide” may be used interchangeably to refer to a polymer of amino acids. A “polypeptide” as contemplated herein typically comprises a polymer of naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). The heterologous polypeptide may include, without limitation, a plant pathogen resistance polypeptide (e.g., CBP60g), a transcription factor, a CAS protein (i.e. Cas9), a reporter polypeptide, a polypeptide that confers resistance to a chemical, or a polypeptide that is involved in the growth or development of plants.

The constructs of the present disclosure may include a heterologous promoter. In some embodiments, the heterologous promoter is the sequence of an endogenous promoter of the heterologous polypeptide that is modified to include uORF, and/or 5′ regulatory sequences (i.e., separately or in combination). In some embodiments, the insert site (whether including a heterologous coding sequence or not) is operably connected to the promoter.

Other aspects and embodiments of the present disclosure comprise vectors including any of the constructs described herein. The term “vector” is intended to refer to a polynucleotide capable of transporting another polynucleotide to which it has been linked. In some embodiments, the vector may be a “plasmid,” which refers to a circular double-stranded DNA loop into which additional DNA segments may be ligated. Another type of vector is a viral vector (e.g., replication defective retroviruses, herpes simplex virus, lentiviruses, adenoviruses and adeno-associated viruses), where additional polynucleotide segments may be ligated into the viral genome. Certain vectors are capable of autonomous replication in a host cell into which they are introduced (e.g., bacterial vectors having a bacterial origin of replication and episomal mammalian vectors). Other vectors can be integrated into the genome of a host cell upon introduction into the host cell, and thereby are replicated along with the host genome, such as some viral vectors or transposons. Plant mini chromosomes are also included as vectors. Vectors may carry genetic elements, such as those that confer resistance to certain drugs or chemicals.

Cells including any of the constructs or vectors described herein are further provided in certain aspects and embodiments of the present disclosure. Suitable “cells” that may be used in accordance with the present invention include eukaryotic cells. Suitable eukaryotic cells include, without limitation, plant cells, fungal cells, and animal cells such as cells from popular model organisms including, but not limited to, Arabidopsis thaliana. In some embodiments, the cell is a plant cell such as, without limitation, a corn plant cell, a bean plant cell, a rice plant cell, a soybean plant cell, a cotton plant cell, a tobacco plant cell, a date palm cell, a wheat cell, a tomato cell, a banana plant cell, a potato plant cell, a pepper plant cell, a moss plant cell, a parsley plant cell, a citrus plant cell, an apple plant cell, a strawberry plant cell, a rapeseed plant cell, a cabbage plant cell, a cassava plant cell, and a coffee plant cell.

The present disclosure further provides for plants including any of the DNA constructs, vectors, or cells described herein are provided. The plants may be transgenic or transiently transformed with the DNA constructs or vectors described herein. In some embodiments, the plant may include, without limitation, a corn plant, a bean plant, a rice plant, a soybean plant, a cotton plant, a tobacco plant, a date palm plant, a wheat plant, a tomato plant, a banana plant, a potato plant, a pepper plant, a moss plant, a parsley plant, a citrus plant, an apple plant, a strawberry plant, a rapeseed plant, a cabbage plant, a cassava plant, and a coffee plant.

The present disclosure further comprises methods for controlling the expression of a heterologous polypeptide in a cell to preserve and/or restore SA-mediated plant immunity. The methods may include introducing any one of the constructs or vectors described herein into the cell. Preferably, the constructs and vectors include a heterologous coding sequence encoding a heterologous polypeptide. As used herein, “introducing” describes a process by which exogenous polynucleotides (e.g., DNA or RNA) are introduced into a recipient cell. Methods of introducing polynucleotides into a cell are known in the art and may include, without limitation, microinjection, transformation, and transfection methods. Transformation or transfection may occur under natural or artificial conditions according to various methods well known in the art, and may rely on any known method for the insertion of foreign nucleic acid sequences into a host cell. The method for transformation or transfection is selected based on the type of host cell being transformed and may include, but is not limited to, the floral dip method, Agrobacterium-mediated transformation, bacteriophage or viral infection, electroporation, heat shock, lipofection, and particle bombardment. Microinjection of polynucleotides may also be used to introduce polynucleotides and/or proteins into cells.

Conventional viral and non-viral based gene transfer methods can be used to introduce polynucleotides into cells or target tissues. Non-viral polynucleotide delivery systems include DNA plasmids, RNA, naked nucleic acid, and nucleic acid complexed with a delivery vehicle, such as a liposome. Methods of non-viral delivery of nucleic acids include the floral dip method, Agrobacterium-mediated transformation, lipofection, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355 and lipofection reagents are sold commercially (e.g., TRANSFECTAM (Promega) and LIPOFECTIN (Invitrogen)). Cationic and neutral lipids that are suitable for efficient receptor-recognition lipofection of polynucleotides include those described in e.g., WO/1991/017424; WO/1991/016024. Delivery can be to cells (e.g., in vitro or ex vivo administration) or target tissues (e.g., in vivo administration).

### Gene Editing

In another embodiment, the CBP60g gene is modified using gene editing technology to modify the CBP60g gene promoter. Such methods can be carried out using any suitable method known in the art, e.g., using a CRISPR-Cas system. The CRISPR-Cas system comprises at least one guide RNA (typically a single guide RNA, or sgRNA), an RNA-guided nuclease (such as Cas9 or Cpf1), and optionally a homologous donor template. A homologous donor template can be used to introduce specific modifications into the genome by homologous recombination.

Many genome modifying systems and corresponding nucleases may be used, for example, without limitations, a homing nuclease polypeptide; a FokI polypeptide; a transcription activator-like effector nuclease (TALEN) polypeptide; a MegaTAL polypeptide; a meganuclease polypeptide; a zinc finger nuclease (ZFN); an ARCUS nuclease; and the like. The meganuclease can be engineered from an LADLIDADG homing endonuclease (LHE). A megaTAL polypeptide can comprise a TALE DNA binding domain and an engineered meganuclease. See, e.g., International Patent Application Publication No. WO 2004/067736 (homing endonuclease); Urnov et al. (2005) Nature 435:646 (ZFN); Mussolino et al. (2011) Nucle. Acids Res. 39:9283 (TALE nuclease); Boissel et al. (2013) Nucl. Acids Res. 42:2591 (MegaTAL). Other useful gene editing systems include Retron Library Recombineering (RLR), NgAgo Protein Gene Editing, FANA Antisense Oligonucleotide (FANA Aso) technology, and NICER.

In some embodiments, the genome modifying system is a CRISPR-Cas system comprising a CRISPR-Cas nuclease and guide polynucleotides, e.g., guide RNAs (gRNAs). In some embodiments, for example, the CRISPR-Cas nuclease is CRISPR-Cas9. See, e.g., Møller, et al. Nucleic Acids Research (2018). In some embodiments, the CRISPR-Cas system is a Type II CRISPR/Cas system (such as a CRISPR/Cas9 system, including, but not limited to Streptococcus pyogenes Cas9 (SpCas9), Streptococcus thermophilus Cas9 (StCas9), Campylobacter jejuni Cas9 (CjCas9) and Neisseria cinerea Cas9 (NcCas9)), a Type I CRISPR/Cas system, a Type III CRISPR/Cas systems, a Type V CRISPR/Cas system (such as Cas12a, e.g., Francisella novicida Cpf1 (FnCpf1), Acidaminococcus sp. Cpf1 (AsCpf1), and Lachnospiraceae bacterium ND2006 Cpf1), and/or comprises a Cas-CLOVER nuclease, a Mini-Cas9 enzyme, or a hfCas12Max nuclease.

The CRISPR-Cas nuclease can be any of a variety of CRISPR-Cas nucleases. CRISPR-Cas nucleases can be derived from a variety of bacterial species. Also suitable for use is a variant CRISPR-Cas nuclease, where the variant is a high-fidelity or enhanced specificity CRISPR-Cas nuclease with reduced off-target effects and robust on-target cleavage. Non-limiting examples of CRISPR-Cas nuclease variants with improved on-target specificity include the SpCas9 (K855A), SpCas9 (K810A/K1003A/R1060A) (also referred to as eSpCas9 (1.0)), and SpCas9 (K848A/K1003A/R1060A) (also referred to as eSpCas9 (1.1)) variants described in Slaymaker et al. Science, 351 (6268): 84-8 (2016), and the SpCas9 variants described in Kleinstiver et al. Nature, 529 (7587): 490-5 (2016) containing one, two, three, or four of the following mutations: N497A, R661A, Q695A, and Q926A (e.g., SpCas9-HF1 contains all four mutations). Also suitable for use, e.g., when fused with a second enzyme with nicking of DNA cleaving activity, is a variant CRISPR-Cas nuclease, where the variant CRISPR-Cas nuclease has reduced or no nucleic acid cleavage activity.

In many embodiments, guide polynucleotides, e.g., gRNAs, are used with a CRISPR-Cas nuclease. In some embodiments, a guide polynucleotide, e.g., gRNA, comprises a sequence having at least 70%, at least 80%, at least 85%, at least 90%, at least 95%, or 100% identity to a sequence at a target site. In some embodiments, the target site is on the genomic DNA of a host cell. In some embodiments, a gRNA library is used with the CRISPR-Cas nuclease. A non-limiting example of a gRNA library is the CRISPR-Cas9 MinLib plasmid library (MinLibCas9 Library, Addgene #164896).

In some embodiments, the single guide RNAs (sgRNAs) is designed to target the CBP60g gene promoter. sgRNAs interact with a site-directed nuclease such as Cas9 and specifically bind to or hybridize to a target nucleic acid within the genome of a cell, such that the sgRNA and the site-directed nuclease co-localize to the target nucleic acid in the genome of the cell. The sgRNAs as used herein comprise a targeting sequence that has homology (or complementarity) to a target DNA sequence at the CBP60g gene promoter, and a constant region that mediates binding to Cas9 or another RNA-guided nuclease. The sgRNA can target any sequence within the CBP60g gene promoter adjacent to a PAM sequence.

The targeting sequence of the sgRNAs may be, e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or, e.g., 15-25, 18-22, or 19-21 nucleotides in length, and shares homology with a targeted genomic sequence, in particular at a position adjacent to a CRISPR PAM sequence. The sgDNA targeting sequence is designed to be homologous to the target DNA, i.e., to share the same sequence with the non-bound strand of the DNA template or to be complementary to the strand of the template DNA that is bound by the sgRNA. The homology or complementarity of the targeting sequence can be perfect (i.e., sharing 100% homology or 100% complementarity to the target DNA sequence) or the targeting sequence can be substantially homologous (i.e., having less than 100% homology or complementarity, e.g., with 1-4 mismatches with the target DNA sequence).

Each sgRNA also includes a constant region that interacts with or binds to the site-directed nuclease, e.g., Cas9. In the nucleic acid constructs provided herein, the constant region of an sgRNA can be from about 70 to 250 nucleotides in length, or about 75-100 nucleotides in length, 75-85 nucleotides in length, or about 80-90 nucleotides in length, or 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length. The overall length of the sgRNA can be, e.g., from about 80-300 nucleotides in length, or about 80-150 nucleotides in length, or about 80-120 nucleotides in length, or about 90-110 nucleotides in length, or, e.g., 90, 91, 92, 93, 94, 95, 96, 97, 98, 99, 100, 101, 102, 103, 104, 105, 106, 107, 108, 109, or 110 nucleotides in length.

It will be appreciated that it is also possible to use two-piece gRNAs (cr:tracrRNAs) in the present methods, i.e., with separate crRNA and tracrRNA molecules in which the target sequence is defined by the crispr RNA (crRNA), and the trans-activating crispr RNA (tracrRNA) provides a binding scaffold for the Cas nuclease.

The sgRNAs can be obtained in any of a number of ways. For sgRNAs, primers can be synthesized in the laboratory using an oligo synthesizer, e.g., as sold by Applied Biosystems, Biolytic Lab Performance, Sierra Biosystems, or others. Alternatively, primers and probes with any desired sequence and/or modification can be readily ordered from any of a large number of suppliers, e.g., ThermoFisher, Biolytic, IDT, Sigma-Aldritch, GeneScript, etc.

The sgRNA and nuclease can be introduced into a cell using any suitable method, e.g., by introducing one or more polynucleotides encoding the sgRNA and the nuclease into the cell, e.g., using a vector such as a viral vector or delivered as naked DNA or RNA, such that the sgRNA and nuclease are expressed in the cell. In some embodiments, the sgRNA and nuclease are assembled into ribonucleoproteins (RNPs) prior to delivery to the cells, and the RNPs are introduced into the cell by, e.g., electroporation. RNPs are complexes of RNA and RNA-binding proteins. In the context of the present methods, the RNPs comprise the RNA-binding nuclease (e.g., Cas9) assembled with the guide RNA (e.g., sgRNA), such that the RNPs are capable of binding to the target DNA (through the gRNA component of the RNP) and cleaving it (via the protein nuclease component of the RNP).

The CRISPR-Cas system typically includes a homologous repair template, or homologous donor template. The template includes a sequence that will be integrated into the genome in the place of a corresponding sequence in the genome, e.g., the sequence present between homologous regions in the template will replace a corresponding sequence present between the corresponding homologous regions in the genome. For example, in some embodiments the sequence in the template will introduce a deletion or an inactivating mutation, or a replacement sequence, into the genomic sequence of the CBP60g gene promoter, thereby modifying CBP60g gene expression and/or activity in the cell. In particular embodiments, the sequence to be introduced is flanked in the template by homology regions, e.g., sequences of from, e.g., 100, 200, 300, 400, 500 or more nucleotides comprising homology to the genomic sequence on either side of the gRNA target sequence.

### Screening

Another aspect of the present application is a method for screening a subject plant for increased immunity to a pathogen when under an environmental stress. In some instances, it can be useful to assess the immune capabilities of plants made according to the methods described above. In general, the method comprises comparing the immune response of a plant produced according to the provided methods when challenged with pathogen exposure under normal conditions and under stress conditions. Plants that have been modified to have sustained SA-mediated immunity to a pathogen will demonstrate reduced signs of pathogen infection under an environmental stress (e.g., heat, drought, and/or salinity) than a control plant. In some embodiments, the method comprises the steps of: incubating a first copy of the subject plant with the pathogen under normal environmental growing conditions for a defined time period, wherein the subject plant comprises a plant made according to the methods provided in this disclosure; incubating a first copy of a control plant with the pathogen under the normal environmental growing conditions for the defined time period, wherein the control plants is the same species as the plant of interest of the third aspect and is susceptible to the pathogen under the environmental stress; incubating a second copy of the subject plant with the pathogen under conditions comprising the environmental stress for the defined time period; incubating a second copy of the control plant with the pathogen under conditions comprising the environmental stress for the defined time period; assessing the first and second copies of the subject plant and the first and second copies of the control plant for characteristics of exposure to the pathogen at the end of the defined time period; and indicating that the subject plant has (i) increased immunity to a pathogen when under an environmental stress if the second copy of the subject plant exhibits fewer and/or reduced characteristics of exposure to the pathogen after the defined time period as compared to the second copy of the control plant after the defined time period, or (ii) does not have increased immunity to a pathogen when under an environmental stress if the second copy of the subject plant exhibits similar characteristics of exposure to the pathogen after the defined time period as compared to the second copy of the control plant after the defined time period.

In some embodiments, the second subject plant having increased immunity to a pathogen when under an environmental stress exhibits similar characteristics of exposure to the pathogen after the defined time period to the first copy of the subject plant and/or the first copy of the control plant after the defined time period.

In some embodiments, the environmental stress is at least one of heat stress, drought, or salinity. In some further embodiments, the heat stress is an ambient temperature that is elevated over a maximum ambient temperature for the plant. In some embodiments, the pathogen is selected from the group consisting of a fungus, a fungal-like organism, a bacterium, a phytoplasma, a protist, a virus, and a viroid.

Another aspect of the present disclosure provides a method for preserving and/or restoring salicylic acid (SA)-mediated immunity in a plant in response to elevated temperature, the method comprising, consisting of, or consisting essentially of modifying the expression of the endogenous CBP60g gene such that salicylic acid (SA)-mediated immunity is preserved and/or restored in the plant.

### Key Sequence of the Disclosure

The following are the cDNA sequences of Arabidopsis thaliana CBP60g and GBPL3.

SEQ ID NO:1. (cDNA; Calmodulin-binding protein 60 g [CBP60g];
Arabidopsis thaliana)

| | |
|---|---|
| 1 | TGGAAGTTTC ACTGCTGCTT CGTCAATACT CATTGCCTTT ATATTTTCCT |
| 51 | CATTCACTCT CTTCCCATTC TCATCTCTCT CTCTCTACCT TTCTCTCACA |
| 101 | CAAAGAGTTT TTTCTTTTAA GACAAAAACT CTTTCAAGGT TTTAAATCTT |
| 151 | TGAAAAAATT CTCTAAACCT AAAAGTGATC AATGAAGATT CGGAACAGCC |
| 201 | CTAGTTTTCA TGGGGGTAGT GGTTACAGTG TCTTTAGAGC TCGTAACTTA |
| 251 | ACATTCAAGA AAGTTGTGAA GAAAGTGATG AGAGACCAGT CAAATAATCA |
| 301 | ATTCATGATT CAGATGGAGA ACATGATAAG AAGAATTGTC CGAGAGGAGA |
| 351 | TACAGCGTAG TCTTCAACCT TTTCTCTCTT CATCATGCGT CTCAATGGAG |
| 401 | CGATCTCGCT CAGAAACGCC ATCGTCTCGC TCACGATTGA AGCTGTGCTT |
| 451 | CATAAACTCG CCGCCATCAT CGATATTCAC GGGGTCCAAG ATCGAAGCTG |
| 501 | AGGATGGTTC TCCGCTTGTG ATCGAGCTCG TGGACGCCAC CACAAACACT |
| 551 | CTAGTTAGTA CGGGACCGTT CTCGTCTTCT CGGGTCGAGC TCGTGCCGCT |
| 601 | GAACGCTGAT TTCACGGAAG AAAGCTGGAC CGTTGAGGGA TTTAATCGGA |
| 651 | ATATTCTCAC GCAACGTGAA GGGAAACGTC CGTTGCTCAC TGGAGACCTA |
| 701 | ACGGTGATGC TTAAAAACGG TGTTGGAGTT ATAACCGGAG ATATAGCTTT |
| 751 | CTCGGATAAC TCGAGCTGGA CTAGGAGTCG GAAGTTCCGG TTAGGTGCTA |
| 801 | AGTTGACCGG AGATGGAGCC GTGGAGGCGA GAAGTGAAGC TTTTGGATGT |
| 851 | AGAGACCAAC GAGGAGAATC TTATAAAAAA CATCATCCTC CGTGCCCCAG |
| 901 | TGATGAGGTT TGGAGACTAG AGAAAATCGC GAAAGATGGA GTTTCGGCAA |
| 951 | CGCGTTTGGC TGAACGCAAG ATTTTAACCG TCAAGGATTT TCGCCGTTTG |
| 1001 | TATACTGTAA ATCGAAATGA GTTACACAAC ATAATTGGTG CAGGGGTCTC |
| 1051 | AAAGAAAACA TGGAACACAA TTGTATCACA TGCCATGGAT TGCGTTTTGG |
| 1101 | ACGAAACAGA GTGTTACATT TACAATGCAA ACACTCCGGG CGTAACACTT |
| 1151 | CTCTTCAACT CTGTTTATGA GTTGATAAGA GTGTCATTCA ATGGCAACGA |
| 1201 | TATCCAAAAC CTTGATCAGC CAATTCTAGA CCAATTAAAG GCCGAAGCTT |
| 1251 | ATCAAAACCT TAACCGCATT ACAGCGGTTA ACGATAGGAC CTTTGTGGGT |
| 1301 | CATCCACAAA GGTCCTTACA GTGCCCGCAA GATCCTGGAT TTGTCGTAAC |
| 1351 | ATGTTCTGGA TCGCAGCACA TCGACTTTCA AGGAAGTTTG GATCCATCAA |
| 1401 | GCTCTTCGAT GGCTCTTTGC CACAAAGCTT CAAGCTCAAC GGTCCACCCT |
| 1451 | GATGTCCTGA TGAGTTTTGA TAACTCATCA ACCGCGAGGT TTCATATCGA |
| 1501 | CAAAAAGTTC TTACCGACTT TCGGAAACAG CTTCAAAGTA AGTGAACTCG |
| 1551 | ATCAAGTACA CGGAAAATCA CAAACTGTTG TGACAAAAGG TTGTATAGAG |
| 1601 | AATAACGAGG AGGATGAGAA CGCGTTTTCT TATCATCACC ATGATGACAT |
| 1651 | GACCTCAAGC TGGTCACCTG GTACGCACCA AGCCGTTGAA ACGATGTTTC |
| 1701 | TTACCGTGTC TGAGACGGAA GAAGCTGGAA TGTTCGATGT TCATTTTGCA |
| 1751 | AACGTTAATT TGGGATCTCC AAGAGCCAGG TGGTGTAAGG TTAAGGCAGC |
| 1801 | TTTCAAGGTT AGGGCAGCTT TTAAGGAAGT CCGGAGACAC ACAACTGCCA |
| 1851 | GAAATCCGAG GGAAGGCTTG TAAGAACTCT TGCCTACCTA TTAAAATCAG |
| 1901 | CGGTTTCTTG CGTTGCACGA TAAATGTGTA TATATATTTG TGAATAGGGT |
| 1951 | TAGATAAATG GATCCCGGCC ATTGAGTTAC GGGGATTAGC CTTTGCGTAT |
| 2001 | ATCAAAGTAT TTTTTTCCCT GGGTAAGTAT AGCTGTGGCA CAGGGTTTGT |
| 2051 | AAGTTGTAAC CATATGCCGT ATAAGAATCA GAGGTTTCTT GTTGTACACG |
| 2101 | AAAGCTATGT ATAAATAAAA GCTATGTATA TATGTTTTTT TCTACCATAA |
| 2151 | CTTTTCAATA ATGGATCTCG GCTATTGATT GTTCTAGACC AATATGATAC |
| 2201 | AT |

SEQ ID NO: 2. (cDNA; Guanylate-Binding Protein-Like 3 [GBPL3];
Arabidopsis Thaliana)

| | |
|---|---|
| 1 | TCTCTCGCGA CGGATCTTCT TAGGTCTCTC TCTTCTCCTC CTTTTCCCCA |
| 51 | CTTTTTCAAA ATCAGCAAAT TTTGGATTTT CAGAATTTGA GTGAACTTAA |
| 101 | AGTAGAAGAC GAATTGTCTG AATTCGAATT TTAAGAAAAA TTTGCAGAGA |
| 151 | TGAGGAGTTT TTTCGGTAGA GGAGGGAAGG ATTCACCGGC TGATTCTGCT |
| 201 | TCTCCGTCAC CGAGATCATA CCCTTCGACG TCTCCGGCGT CTTCGTCTGC |
| 251 | TGTGACGGGA CCACCGAGAC CTATTCGTTT AGTGTATTGT GATGAGAAAG |
| 301 | GGAAGTTTCG TATGGACCCT GAAGCTGTTG CTACTTTGCA ACTCGTTAAG |
| 351 | GAGCCAATTG GTGTTGTTTC GGTTTGTGGT AGGGCTCGTC AAGGAAAGAG |
| 401 | CTTTATTTTG AATCAGCTTC TTGGACGCAG TAATGGGTTT CAAGTAGCAT |
| 451 | CAACACACAA GCCGTGTACC AAGGGGCTTT GGTTGTGGAG TTCTCCTATA |
| 501 | AAAAGAACAG CTCTTGACGG AACTGAATAC AATCTTTTGT TATTAGATAG |
| 551 | TGAAGGAATT GATGCTTACG ACCAAACTGG TACCTACAGC ACTCAAATAT |
| 601 | TCTCATTAGC TGTTCTTTTG TCAAGCATGT TCGTGTATAA TCAGATGGGA |
| 651 | GGCATTGACG AAGCTTCACT GGATCGCCTT TCTCTTGTCA CTCAAATGAC |
| 701 | AAAGCATATC CGCGTAAAAG CTTCTGGAGG GACGAGCTCA CGTTCTGAAC |
| 751 | TTGGGCAATT TTCTCCCATC TTTGTCTGGC TGTTGAGGGA CTTTTATTTG |
| 801 | GATCTAGTGG AAGATAATCG GAAGATTAGC CCACGTGACT ATTTAGAAAT |
| 851 | TGCCTTAAGG CCAGTTCAAG GTTCGGGAGG GGATATTGGT GCTAAAAACG |
| 901 | AGATCCGAGA TTCAATCCGT GCTCTTTTTC CGGACAGAGA GTGCTTTACC |
| 951 | CTTGTAAGGC CTCTGAACAA TGAGAAGGAC CTGCAGAGAC TGGATCAAAT |
| 1001 | TTCGTTGGAA AAATTGAGAC CTGAATTCGG TGCTGGCCTG GATGCATTTA |
| 1051 | CCAAGTTTGT TTTTGAGAAG ACGAGGCCCA AGCAATTAGG GGGTACTGTG |
| 1101 | ATGACTGGCC CTATTCTTGT CGGTATTACA CAGTCTTATC TGGATGCTTT |
| 1151 | GAATAATGGT GCCGTGCCAA CAATAACCTC ATCTTGGCAG AGTGTCGAAG |
| 1201 | AAACTGAGTG TCGAAGAGCA TATGATTCTG GTGTAGAAGC CTATATGGCT |
| 1251 | GCCTTTGACC AATCAAAAGC TCCGGAAGAA GGTGCACTGA GGGAAGAACA |
| 1301 | TGAAGAAGCA GTTCGAAAAG CCTTGGCTAT ATTTAACTCT AATGCTGTAG |
| 1351 | GGAATGGTTC AGCAAGAAAA AAATTCGAGG ATCTTCTCCA CAAGGACTTA |
| 1401 | AAGAAAAAGT TTGAGGATTA CAAGAAGAAC GCTTTTATGG AGGCAGATTT |
| 1451 | GCGATGTACG AGTACTATCC AGCGTATGGA AAAGCAGCTT AGAGCAGCTT |
| 1501 | GCCATGCCTC TAATGCCAAT ATGGATAATG TTGTCAAGGT CCTAGAAGCT |
| 1551 | CGTTTGGCAG AATATGAGGC ATCATGCCAT GGCCCGGGGA AATGGCAGAA |
| 1601 | ACTTTCTGTA TTTTTGCAAC AAAGCTTGGA AGGACCTATA TATGATCTCA |
| 1651 | CCAAAAGGCT TATAGATAGC ATCGCTATAG AGAAGAATTC CCTTGCAATG |
| 1701 | AAATTTCGTT CTGTTGAAGA TGCGATGAAA CATTTAAAAC AGCAGTTGGA |
| 1751 | TGATAGTGAG AGATACAAGT TGGAATACCA GAAACGTTAT GATGAATCCA |
| 1801 | ACAATGACAA GAAGAAGCTG GAAGATATAT ACAGAGAGCG CATAACTAAA |
| 1851 | CTACAGGGGG AGAATAGTTC GCTGAACGAG CGATGCTCTA CGTTGGTTAA |
| 1901 | AACCGTGGAA TCTAAAAAAG AAGAAATCAA AGAATGGATT AGAAATTATG |
| 1951 | ACCAGATTGT TTTGAAGCAG AAAGCTGTTC AAGAACAGCT TAGTTCTGAA |
| 2001 | ATGGAAGTCC TTAGGACAAG AAGTACTACT TCTGAAGCTA GGGTTGCTGC |
| 2051 | AGCTAGAGAA CAAGCCAAAT CTGCTGCAGA AGAGACCAAG GAATGGAAAA |
| 2101 | GGAAGTATGA TTATGCTGTG GGAGAGGCTA GATCTGCTCT TCAAAAGGCT |
| 2151 | GCTTCAGTGC AAGAACGTTC TGGCAAGGAA ACACAGTTGA GGGAAGATGC |
| 2201 | TCTTAGGGAG GAATTTAGTA TCACTTTAGC TAATAAGGAT GAAGAAATTA |
| 2251 | CGGAGAAGGC TACAAAACTT GAGAAGGCAG AGCAATCTCT AACGGTTCTA |
| 2301 | AGATCAGATT TGAAGGTGGC TGAGTCAAAA CTTGAAAGCT TTGAGGTAGA |
| 2351 | GTTAGCATCA CTAAGATTAA CATTAAGTGA AATGACTGAT AAGTTGGACA |
| 2401 | GTGCCAACAA AAAGGCTCTA GCATATGAAA AGGAGGCTAA TAAGTTGGAG |
| 2451 | CAAGAGAAAA TCCGTATGGA ACAAAAGTAT CGATCTGAGT TTCAACGCTT |
| 2501 | CGATGAAGTC AAAGAGAGAT GTAAGGCAGC TGAAATAGAA GCTAAACGCG |
| 2551 | CTACTGAACT GGCAGACAAA GCTCGAACTG ATGCTGTCAC GTCGCAAAAG |
| 2601 | GAGAAAAGTG AGAGTCAGAG GTTGGCGATG GAAAGACTTG CTCAGATCGA |
| 2651 | ACGAGCTGAG AGGCAAGTCG AAAACTTAGA GAGACAGAAG ACTGACTTGG |
| 2701 | AGGATGAACT GGACAGACTT CGTGTGTCTG AGATGGAGGC TGTCTCCAAA |
| 2751 | GTTACAATCT TAGAAGCAAG AGTTGAAGAA CGAGAGAAAG AAATCGGGTC |
| 2801 | ATTGATAAAG GAAACCAACG CACAGAGGGC TCATAACGTG AAGTCACTTG |
| 2851 | AAAAGCTGTT GGATGAAGAG CGTAAGGCTC ATATAGCTGC AAATCGAAGA |
| 2901 | GCCGAAGCTC TTTCTCTTGA GCTGCAAGCT GCACAGGCAC ACGTTGATAA |
| 2951 | TCTTCAGCAA GAATTAGCTC AAGCAAGGTT AAAAGAAACC GCACTTGACA |
| 3001 | ACAAAATCAG AGCTGCGAGT TCCTCACATG GGAAGCGGAG TAGATTTGAA |
| 3051 | GATGTTGTTG ATATGGACAT TGGAGAAGGA AGTGATAGAA TCTTGAGGAC |
| 3101 | TAATAAACGA GCTCGAAGCA CGAGAGGAGA TGATCACGGT CCTACTGATG |
| 3151 | AAGGCGACGA GGACTTTCAA AGTCATCAAG ACAATGGCGA GGAGGAAGAA |
| 3201 | GAAGAAGATT ACAGGAAGCT TACTGTGCAG AATCTAAAGC ATGAGCTGAC |
| 3251 | CAAGTACGAT TGTGGACATT TGTTATTGAA CAGAGGCCAT CAAAACAAGA |
| 3301 | AAGAGATTCT TGCGTTGTAT GAAGCTCATG TTCTTCCCAA GAAAGCTTTA |
| 3351 | GCGAGAGAGG AAGAGAGAAA GAAACAGAGA GAAGTCACTT CTTCTTAGAG |
| 3401 | GACAAGACTC TGTACAGAGT AAGGAGATAA ACATGTTTAA TCTCAAAAGG |
| 3451 | TTTCGATCTC TAGAGATTTA GTAAGAGTTG GAATTAGAAA AGGCTCGTAT |
| 3501 | TGTTTCTCTC TTTTTATCGG TTTTACCGGT TTACCTGGTA CTGCAGAAAG |
| 3551 | TAAACCGAAT TAGCCATTTG TATTGAATCT GAGGGTTTAG GTTTTTTTAT |
| 3601 | GAGTGTTTTT TTTTTGTGTG TTTGGTATTT AATTTGTGTG GAGTTTGAGT |
| 3651 | CGGTTTGGTG TTTTGACTTT TTGATTTTTA GATTCAAGTC TTTTTCAGAA |
| 3701 | TTCTGTTACT CGTTTGAGAT CGACCGTTTG CTTTGAATTT TACTCTTCAA |
| 3751 | TCCTAAGTCA GCAGTATTGA CTAATTGCGT TTAAAGGAAT AATTCTAAGA |
| 3801 | TTATGTTAAC TTGGTGATAG GCT |

SEQ ID NO: 61. (uORFS_{TBF1})

| | |
|---|---|
| 1 | AACAGCATCC GTTTTTATAA TTTAATTTTC TTACAAAGGT AGGACCAACA |
| 51 | TTTGTGATCT ATAAATCTTC CTACTACGTT ATATAGAGAC CCTTCGACAT |
| 101 | AACACTTAAC TCGTTTATAT ATTTGTTTTA CTTGTTTTGC ACATACACAC |
| 151 | AAAAATAAAA AAGACTTTAT ATTTATTTAC TTTTTAATCA CACGGATTAG |
| 201 | CTCCGGCGAA GTATGGTCGT CGTCTTCATC TTCTTCCTCC ATCATCAGAT |
| 251 | TTTTCCTTAA ATGGAAGAAA CCAAACGAAA CTCCGATCTT CTCCGTTCTC |
| 301 | GTGTTTTCCT CTCTGGCTTT TATTGCTGGG ATTGGGAATT TCTCACCGCT |
| 351 | CTCTTGCTTT TTAGTTGCTG ATTCTTTTTC CTTCGACTTT CTATTTCCAA |
| 401 | TCTTTCTTCT TCTCTTTGTG TATTAGATTA TTTTTAGTTT TATTTTTCTG |
| 451 | TGGTAAAATA AAAAAAGTTC GCCGGAG |

The following Examples are provided by way of illustration and not by way of limitation.

### EXAMPLES

The following examples are offered to illustrate, but not to limit the claimed invention. Example 2 was included in U.S. Provisional Application No. 63/523,390, filed Jun. 27, 2023, and describe the initial investigation of SA-mediated immunity in plants at elevated temperatures. Examples 3-12 describe the updated investigation of SA-mediated immunity in plants at elevated temperatures. Example 1 describes the methods used both in the initial and the updated investigations unless otherwise specified.

### Example 1: Materials and Methods

Plant materials: Arabidopsis thaliana plants were grown in soil (2:1 “Arabidopsis mix”: perlite) covered with or without standard Phifer glass mesh for 3-4 weeks at 21° C.-23° C. and 60% relative humidity under a 12 h light/12 h dark regimen (100±10 μmol m-2 s-1). Accessions, mutants and transgenic lines are outlined in Table 1. All experiments with 35S::CBP60g were performed with line #17, unless otherwise specified.

Rapeseed (Brassica napus) cultivar Westar, tomato (Solanum lycopersicum) cultivar Castlemart, and tobacco (Nicotiana tabacum) cultivar Xanthi seeds were grown in “Arabidopsis Mix” soil supplemented with 1 g/L of 20-20-20 general purpose fertilizer (Peters Professional). After 2 days of imbibition, plants were grown in growth chambers (20° C./18° C., 16 h day/8 h night for rapeseed; 23° C./23° C.; 12 h day/12 h night for tomato and tobacco) for 4-7 weeks.

Rice (Oryza sativa) cultivar Nipponbare seeds were germinated on wet filter paper in petri dishes and 4- to 5-day-old seedlings were transplanted to Redi-earth soil. Seedlings were grown at 28° C. (16 h day/8 h night) for 4-5 weeks.

TABLE 1
Plant materials used.

| Plant Material | Species | Reference |
|---|---|---|
| Col-0 | Arabidopsis | Arabidopsis Biological |
| | thaliana | Resource Centre (ABRC) |
| | | at The Ohio State |
| | | University. |
| Ler | A. thaliana | ABRC |
| C24 (CS28127) | A. thaliana | ABRC |
| 35S::CBP60g OE-16 | A. thaliana | Wan et al., 2012⁵² |
| 35S::CBP60g OE-17 | A. thaliana | Wan et al., 2012⁵² |
| 35S::uORF_{STBF1}-CBP60g #b5, | A. thaliana | This study |
| b14 | | |
| 35S::ICS1 | A. thaliana | This study |
| 35S::TGA1-4myc | A. thaliana | This study |
| camta2/3 | A. thaliana | Kim et al., 2013⁵³ |
| 35S::WRKY75 | A. thaliana | Zhang et al., 2018 |
| 35S::EDS1 | A. thaliana | Feys et al., 2005 |
| 35S::PAD4 | A. thaliana | Xing et al., 2006⁵⁶ |
| bsmt1 | A. thaliana | Attaran et al., 2009 |
| npr1 S11D/S15D | A. thaliana | Saleh et al., 2015 |
| npr3-2 npr4-2 | A. thaliana | Ding et al., 2018 |
| pCBP60g (1788 bp; −1800 | A. thaliana | Wan et al., 2012⁵² |
| to −14 from ATG):GUS | | |
| npr1-6 | A. thaliana | Huot et al., 2017 |
| NPR1pro::NPR1-EYFP | A. thaliana | Huot et al., 2017 |
| MED16pro::MED16-3FLAG | A. thaliana | Wang et al., 2015⁵⁸ |
| 35S::PHYB^{Y276H} | A. thaliana | Jones et al., 2015⁵⁹ |
| BdELF3-OE | A. thaliana | Jung et al., 2020 |
| 35S::CDK8-myc | A. thaliana | Zhu et al., 2014⁶⁰ |
| 35S::EGFP-GBPL3 | A. thaliana | Huang et al., 2021 |
| Castlemart | Solanum | Tomato Genetics |
| | lycopersicum | Resource Center (TGRC) |
| | | at UC Davis |
| Nipponbare | Oryza sativa | USDA Agricultural |
| | | Research Service |

Generation of constructs and transgenic lines: To generate transgenic Arabidopsis harboring 35S::uORFs_{TBF1}-CBP60g, 35S::TGA1-4myc, or 35S::SARD1, genomic DNA (CBP60g, TGA1) or coding sequences (SARD1) were amplified and ligated into pENTR D-TOPO (Invitrogen). To clone TBF1 uORF sequence, PCR-amplified uORFs_{TBF1} (Xu, 2017) amplicon was ligated into pENTR-ArCBP60g using HiFi DNA Assembly (New England Biolabs). The uORFs_{TBF1}-CBP60g, TGA1 or SARD1 construct was subcloned to pGWB517 through Gateway Cloning (Invitrogen). Plasmids carrying gene constructs were transformed into Agrobacterium tumefaciens GV3101, which was used for Arabidopsis transformation by floral dipping42. T1 plants were selected on half-strength Murashige and Skoog medium supplemented with hygromycin (35 mg/L) and 1% sucrose. Homozygous T2 and T3 transgenic plants were analyzed. Plasmids carrying gene constructs were transformed into Agrobacterium tumefaciens strain GV3101. Transformed Agrobacterium harboring 35S::uORFs_{TBF1}-CBP60g or 35S::TGA1-4xmyc plasmids was used for Arabidopsis transformation by floral dipping. T1 plants were selected on half-strength Murashige and Skoog medium supplemented with hygromycin (35 μg/ml) and 1% sucrose. Homozygous T2 and T3 transgenic plants were used for analyses.

To generate 35S::ICS1 plants, the ICS1 cDNA (SEQ ID NO: 3) was amplified from RNA extracted from infected Arabidopsis leaves and ligated into pCR Blunt TOPO (Invitrogen). Full-length cDNA with chloroplast transit sequence was confirmed and the 35S::ICS1 construct was subcloned into pCAMBIA3301 modified to remove the GUS reporter and to include a C-terminal V5-HexHis tag (Invitrogen) resulting in pSM200-1. pSM200-1 was transformed into A. tumefaciens GV3101 and used to transform Arabidopsis eds 16-1 mutant by floral dipping (Clough, 1998). T1 plants were selected for glucosinolate tolerance using Finale and surviving plants were selfed and tested for presence of the insert using PCR. Homozygous T4 transgenic plants were analyzed.

To generate transgenic rapeseed harboring 35S::AIC BP60g-myc, the AtCBP60g coding sequence, amplified from Arabidopsis cDNA, or the corresponding genomic sequence was cloned into pGWB517 through Gateway reaction (Invitrogen). The binary vector was introduced into A. tumefaciens GV3101 by electroporation. Brassica napus cultivar Westar were transformed using Agrobacterium-mediated method (Zhou, (2002). After 7-day explant-recovery period following co-cultivation on MS medium with benzyladenine (3 mg/l), and Timentin antibiotic (300 mg/l) to eliminate Agrobacterium, putative transformants with roots (TO) were transferred to soil. Genomic DNA was extracted from young leaves using cetyltrimethylammonium bromide (CTAB) method and used for PCR detection of transgene. Two primer pairs for the hygromycin phosphotransferase (HPT) and AtCBP60g genes in the transgene were used to assess transformation. About ten TO transgenic lines were used to produce T1 transgenic plants by self-pollination. RT-qPCR was used to screen for independent T1 transgenics that robustly expressed the AtCBP60g transcript. 35S::AtCBP60g #1-12 was derived from the cDNA construct, whereas 35S::AtCBP60g #2-11 was derived from the genomic DNA construct.

PCR primers are listed in Table 2 and sequences were confirmed by Sanger sequencing.

TABLE 2
Oligonucleotide primers.

| | | | SEQ | |
|---|---|---|---|---|
| Target genes | Sequence (5'-3') | Purpose | ID NO: | Reference |
| CBP60g | caccATGAAGATTCGGAACAGCC | Cloning | 4 | This study |
| (At5g26920) | TTACAAGCCTTCCCTCGGATTTC | | 5 | |
| uORF_{TBF1} | CGCGGCCGCCCCCTTCACC | Cloning | 6 | This study |
| | AACAGCATCCGTTTTTATAA | | | |
| | GGGCTGTTCCGAATCTTCAT | | 7 | |
| | CTCCGGCGAACTTTTTTTAT | | | |
| TGA1 (At5g65210) | CACCATGAATTCGACATCGACACAT | Cloning | 8 | This study |
| | TTTG | | | |
| | CGTTGGTTCACGATGTCGAG | | 9 | |
| PP2AA3 | GGTTACAAGACAAGGTTCACTC | qRT-PCR | 10 | Huot et al., |
| (At1G13320) | CATTCAGGACCAAACTCTTCAG | qRT-PCR | 11 | 2017 |
| CBP60g | TCGTGGACGCCACCACAAACA | qRT-PCR | 12 | Kim et al., |
| (At5g26920) | TCAGCGTTCAGCGGCACGAG | qRT-PCR | 13 | 2017; 2020 |
| ICS1 (At1g74710) | ACTTACTAACCAGTCCGAAAGACGA | qRT-PCR | 14 | Huot et al., |
| | ACAACAACTCTGTCACATATACCGT | qRT-PCR | 15 | 2017 |
| EDSA (At3g48090) | TCGCTTTCCAACCATCTTTCTC | qRT-PCR | 16 | This study |
| | CTACTCCTCACAGCCATTTCC | qRT-PCR | 17 | |
| PAD4 (At3g52430) | ATCTTCTCCGCCGTCATTCC | qRT-PCR | 18 | Kim et al., |
| | CACGTGGCAGAAGTTGTGTG | qRT-PCR | 19 | 2017; 2020 |
| WRKY75 | GTTCCCTAGGAGTTACTATAGGTG | qRT-PCR | 20 | This study |
| (At5g1308) | TTTCGGTGGATTTCTCGATGG | qRT-PCR | 21 | |
| BSMT1 (At3g11480) | CTAGTCTTTGAGGGTCTTGTG | qRT-PCR | 22 | This study |
| | TGAGCATTGGTTCACTAACAG | qRT-PCR | 23 | |
| GUS | ACGGGAAAGGACTGGAAGAG | qRT-PCR | 24 | This study |
| | ATGGTTGCGGATTTCCTTGG | qRT-PCR | 25 | |
| SlPR1b | CTTGCGGTTCATAACGATGC | qRT-PCR | 26 | Kusajima |
| (Solyc09g00701) | TAGTTTTGTGCTCGGGATGC | qRT-PCR | 27 | et al., 2017 |
| SlARD2 | TGTTCATCAGTGTGCTAGTG | qRT-PCR | 28 | Pombo et |
| (Solyc01g104170) | GCTGTCCTTCCTTCTGAATC | qRT-PCR | 29 | al., 2017 |
| OsPR1b | ACGCCTTCACGGTCCATAC | qRT-PCR | 30 | Fang et al., |
| (Os01g0382000) | AAACAGAAAGAAACAGAGGGAGTAC | qRT-PCR | 31 | 2015 |
| OsUBC | CCGTTTGTAGAGCCATAATTGCA | qRT-PCR | 32 | Jain et al., |
| (Os02g0634800) | AGGTTGCCTGAGTCACAGTTAAGTG | qRT-PCR | 33 | 2006 |
| TA3 retrotransposon | CTGCGTGGAAGTCTGTCAAA | ChIP-qPCR | 34 | Yamaguchi |
| | CTATGCCACAGGGCAGTTTT | ChIP-qPCR | 35 | et al., 2014 |
| CBP60g as1 (1.1 kb, | GGGGTCAATGTACTTCTAGTTGA | ChIP-qPCR | 36 | This study |
| P1) | AAACTAGCCAAAACAGCCGT | ChIP-qPCR | 37 | |
| CBP60g CDS (P2) | GAAGATTCGGAACAGCCCTA | ChIP-qPCR | 38 | This study |
| | TCCGAAAATAAACCGGAAAA | ChIP-qPCR | 39 | |
| TZF1 CDS | GCAGTGGACCAAAGAGCAAT | ChIP-qPCR | 40 | This study |
| (At3g55980) | CAAGATCATCACAAGCAGCGA | ChIP-qPCR | 41 | |
| NPR1-P1 | AATGTAAACCGTGGGACGAG | ChIP-qPCR | 42 | Huang et |
| | TAAGAATCGGCGAATCCATC | ChIP-qPCR | 43 | al., 2021 |

Agrobacterium-mediated transient expression in rapeseed and tobacco: For transient expression in rapeseed, Agrobacterium GV3101 harboring 35S::mRFP-4myc or 35S::AtCBP60g-4myc was grown in Luria-Bertani (LB) medium, resuspended in infiltration buffer (10 mM MES [pH 5.7], 10 mM MgCl₂, and 500 μM acetosyringone) at OD600=0.1, and infiltrated to the first and second true leaves of rapeseed plants using a needleless syringe. For transient expression in tobacco (Nicotiana tabacum), Agrobacterium GV3101 harboring 35S::eGFP-GBPL3 or 35S::mRFP-MED15-flag was grown in LB medium, resuspended in the same infiltration buffer at OD600=0.1, and infiltrated to fully expanded leaves of tobacco plants using a needleless syringe. Agroinfiltrated rapeseed or tobacco plants were incubated for 2-3 days at 21-23° C. before experiments.

Temperature conditions: Based on previous studies (Huot, 2017a; Mang, 2012; Yan, 2019; Hammoudi, 2018), Arabidopsis plants were acclimated at 23° C. (ambient) or 28° C. (elevated) for 24 h before chemical treatment and/or 48 h before pathogen infiltration, unless otherwise specified. Four- to 5-week-old rapeseed plants were incubated at ambient (23° C.) or elevated temperatures (28° C.) for 48 h before pathogen infiltration or chemical treatments. Four- to 5-week-old tomato plants were incubated at ambient (23° C.) or elevated temperatures (28° C.-32° C.) for 48 h before chemical treatments. Five-week-old rice plants were incubated at ambient (28° C.) or elevated temperatures (35° C.) before chemical treatments. Four- to 7-week-old tobacco plants were incubated at ambient (23° C.) or elevated temperatures (28° C.) for 48 h before chemical treatments. All plants were grown with a 12 h day/12 h night cycle, except for rice and rapeseed, which was grown with a 16 h day/8 h night cycle.

Growth and developmental phenotyping: For growth biomass measurements, above-ground parts of 4- or 6-week-old pre-flowering plants were weighed, and representative plants were photographed. For flowering time measurements, the first instance of floral appearance for each individual plant was recorded.

BTH and flg22 treatments: Arabidopsis plants were infiltrated or sprayed with mock (0.1% DMSO), benzo (1,2,3) thiadiazole-7-carbothioic acid-S-methyl ester (BTH, Chem Service Inc.; 100 μM, 0.1% DMSO) or flg22 peptide (EZBiolab, 200 nM in 0.1% DMSO). For tomato or rapeseed, 50 (rapeseed) or 100 μM (tomato) of BTH solution (0.02% Silwet L-77 and 0.1% DMSO) or solvent control was sprayed. Plants were further incubated for 24 h. For rice, 200 μM of BTH solution (0.1% Silwet L-77 and 0.1% DMSO) or solvent control was sprayed. Rice plants were further incubated for 24 h and their 4th leaves were used for analyses. were infiltrated or sprayed with mock (0.1% DMSO), benzo (1,2,3) thiadiazole-7-carbothioic acid-S-methyl ester (BTH, Chem Service Inc.; 100 μM, 0.1% DMSO) or flg22 peptide (EZBiolab, 200 nM in 0.1% DMSO). For tomato or rapeseed, 50 (rapeseed) or 100 μM (tomato) of BTH solution (0.02% Silwet L-77 and 0.1% DMSO) or solvent control was sprayed. Plants were further incubated for 24 h. For rice, 200 μM of BTH solution (0.1% Silwet L-77 and 0.1% DMSO) or solvent control was sprayed. Rice plants were further incubated for 24 h and their 4th leaves were used for analyses.

Basal disease resistance assay: Plants were infiltrated with 0.5 to 1.5×10⁶ Colony Forming Units (CFU) ml-1 (OD600=0.0005; for Arabidopsis) or 0.5 to 1.5×10⁵ CFU mL-1 (OD600=0.00005; for rapeseed) of Pst DC3000, 0.5 to 1.5×10⁸ CFU mL-1 of Pst DC3000 ΔhrcC (OD600-0.05; for Arabidopsis) or 0.5 to 1.5×10⁶ CFU ml-1 of P. syringae (Ps) pv. tabaci 11528 (for tobacco) as described previously (Huot, 2017a). Plants were returned to growth chambers at the appropriate temperature and 60% relative humidity. Bacterial levels were measured as previously described (Huot, 2017a; Katagiri, 2002).

Effector-triggered immunity (ETI) assay: Plants were dipped in 0.5 to 1.5×10⁸ CFU ml⁻¹ of Pst DC3000 (avrPphB) (Simonich, 1995) and Pst DC3000 (avrRps4) (Hinsch, 1996) (OD600=0.05) as described previously (Wang, 2009; Katagiri, 2002). Plants were left at room temperature for 1 h with a cover dome to maintain high humidity and then returned to the growth chamber without covering at either 23° C. or 28° C. (60% relative humidity). Bacterial growth was measured as described in the previous section.

Gene expression analyses: RNA extraction and qPCR analyses were performed as described previously (Huot, 2017a). Twenty to 60 mg of fresh leaf tissues were flash-frozen in liquid nitrogen and ground using a TissueLyser (Qiagen). Plant RNA was extracted using a Qiagen Plant RNeasy Mini Kit following the manufacturer's protocol, including on-column DNase I digestion. cDNA was synthesized by adding 100-300 ng of RNA to a solution of oligo-dT primers, dNTPs and M-MLV reverse transcriptase (Invitrogen). ˜1.5 ng of cDNA was mixed with the appropriate primers (Table 2) and SYBR® master mix (Applied Biosystems, Foster City, California). Quantitative PCR (qPCR) was run on a 7500 Fast Real-Time PCR system or QuantStudio 3 Real-Time PCR system (Applied Biosystems, Foster City, California), with 2-4 biological replicates (and 3 technical replicates for each biological replicate) per experimental treatment. StepOnePlus™ (Applied Biosystems, Foster City, California) was used for data acquisition and analysis. Gene expression values were calculated as described previously (Huot, 2017a) with the following internal controls: PP2AA3 (Arabidopsis), SIARD2 (tomato), OsUBC (rice), NtAct (tobacco) and BnaGDI1 (rapeseed). RT-qPCR primer sequences are listed in Table 2.

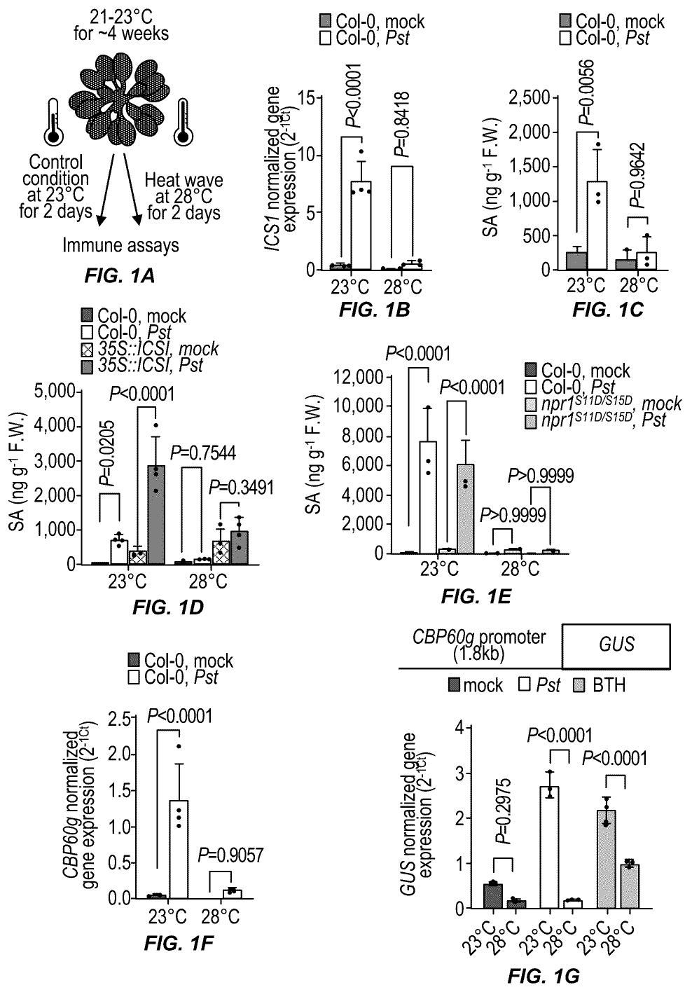

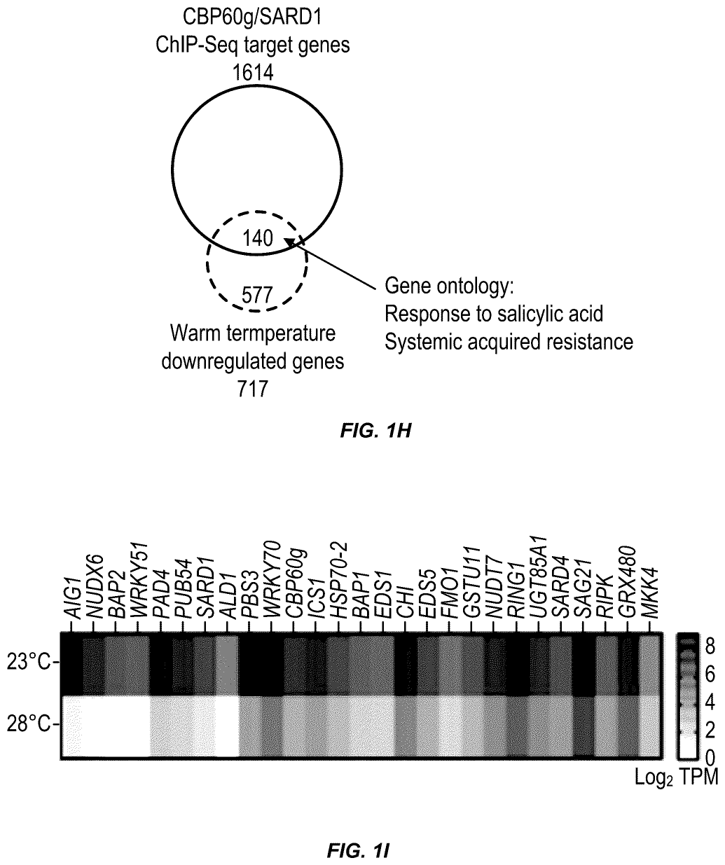

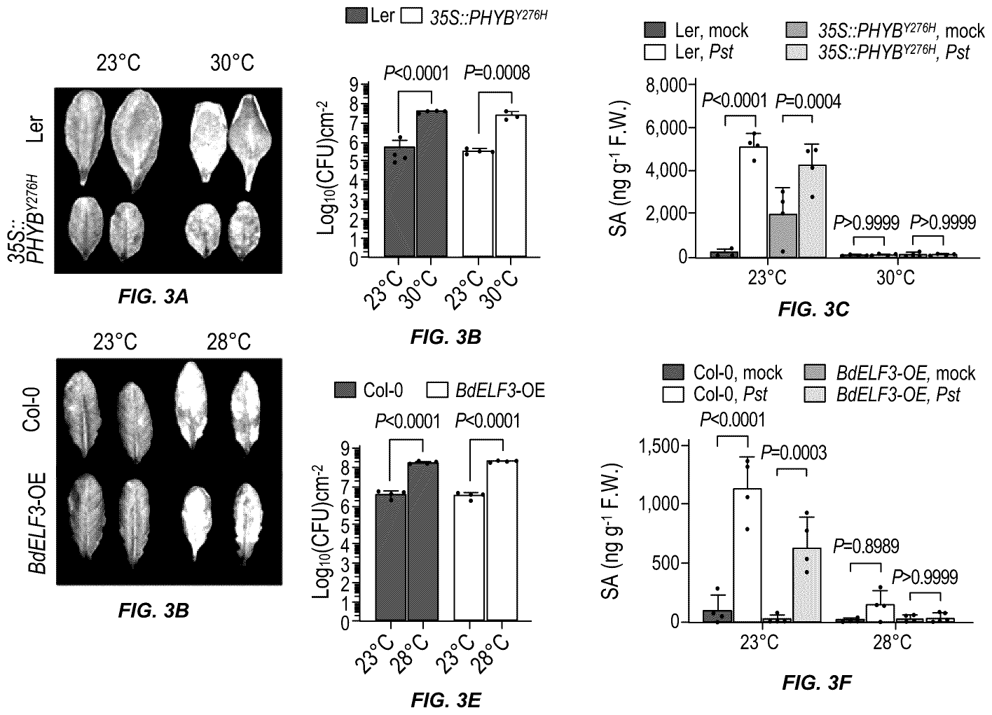

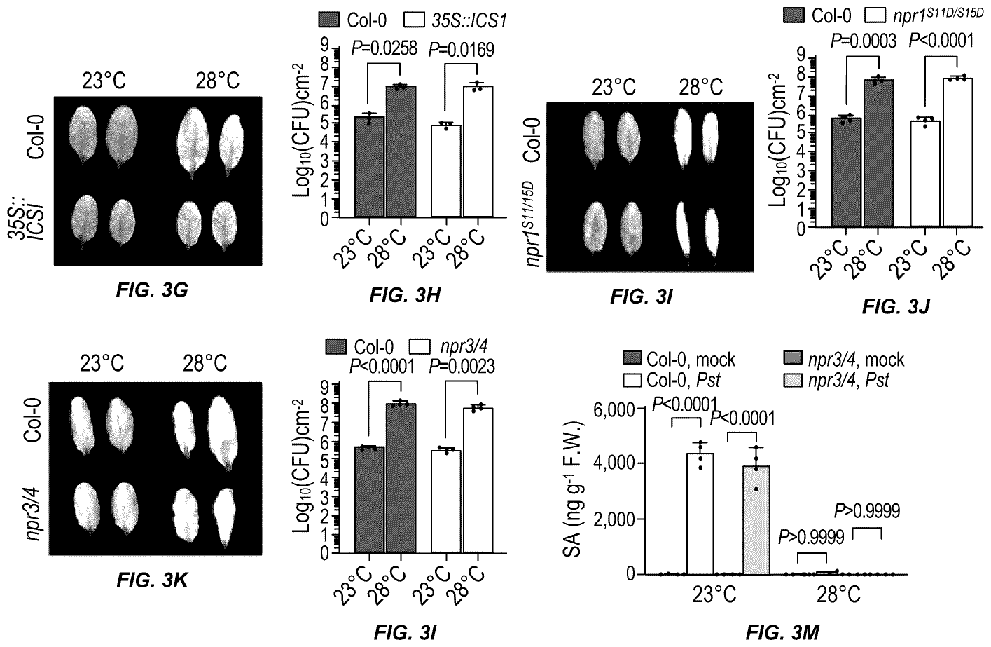

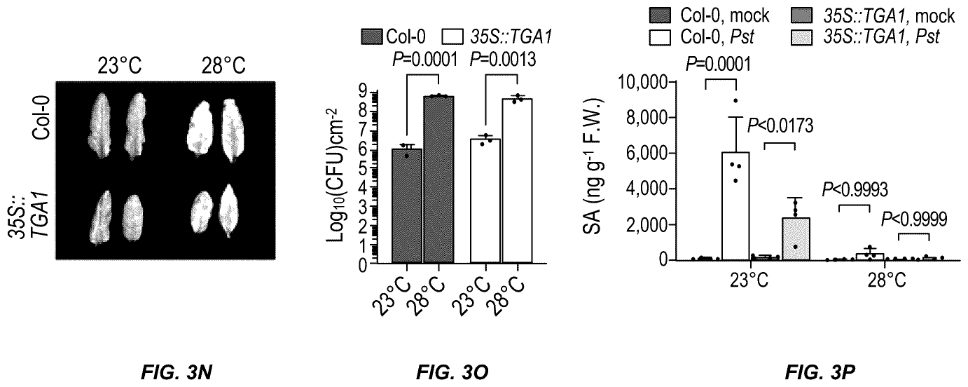

Transcriptome analyses: For RNA-Seq in FIG. 1, Arabidopsis Col-0 plants were inoculated with mock (0.25 mM MgCl₂) or Pst DC3000 suspension, and then incubated at 23° C. or 30° C. for 24 h. For RNA-Seq in FIG. 3, Arabidopsis Col-0 and 35S::CBP60g were inoculated with Pst DC3000 suspension, and then incubated at 23° C. or 28° C. for 24 h. Total RNA was extracted as described above. RNA samples for each treatment were checked for quality and cDNA libraries were prepared, as described previously (Huot, 2017a). All 12 libraries per experiment were pooled in equimolar amounts for multiplexed sequencing. Pools were quantified using the Kapa Biosystems Illumina Library Quantification qPCR kit, and loaded on one lane (FIG. 1) or two lanes (FIG. 3) of Illumina HiSeq 4000 Rapid Run flow cells. RNA-sequencing and analyses were performed as described previously (Huot, 2017a). For FIG. 1, results were filtered for Pst DC3000-induced or -repressed genes using a pathogen/mock fold change >2. Temperature-downregulated, neutral and upregulated target genes were analyzed for Gene Ontology (GO) enrichment using the Database for Annotation, Visualization and Integrated Discovery (DAVID; david.ncifcrf.gov/; Huang, 2009). For FIG. 3, results were further filtered for genes with RPKM values above 1 and 23° C./28° C. RPKM ratios with at least two-fold change. Filtered genes were grouped into four clusters. Cluster 1 had genes more downregulated at 28° C. in Col-0 (i.e. Col/35S::CBP60g ratios of 23° C./28° C. RPKM values >2). Cluster 2 had genes more upregulated at 28° C. in Col-0 (i.e. Col/35S::CBP60g ratios of 23° C./28° C. RPKM values <0.5). Cluster 3 had genes similarly downregulated, while Cluster 4 had genes similarly upregulated in Col-0 and 35S::CBP60g, respectively (i.e. Col/35S::CBP60g ratios of 23° C./28° C. RPKM values between 2 and 0.5). GO enrichment analyses were also conducted using DAVID (Huang, 2009).

Hormone profiling: Plant hormones were extracted and quantified using a previously described protocol (Huot, 2017a), with minor modifications. Methanolic extraction was performed with ABA-d6, SA-d4 or SA-13C6 as an internal control. Filtered extracts were analyzed using an Acquity Ultra Performance Liquid Chromatography (UPLC) system coupled to a Quattro Premier XE MS/MS (Waters Corporation, Milford, MA) or a 1260 infinity High Performance Liquid Chromatography (HPLC) system coupled to a 6460 Triple Quadrupole mass spectrometer (Agilent). Column temperature was set at 40° C. with a 0.4 mL/min flow rate and a gradient of mobile phases water+0.1% formic acid (A) and methanol (B) was used as follows: 0-0.5 min 2% B; 0.5-3 min 70% B; 3.5-4.5 100% B; 4.51-6 min 2% B; followed by additional 1 min for equilibration. Eluted analytes were introduced into Agilent jet stream electro spray ionization (AJS-ESI) ion source and analyzed in negative ion mode with delta EMV (−) of 200. The following parameters were used for the mass spectrometer source: gas temperature, 300° C.; gas flow, 5 L/min; nebulizer, 45 psi; sheath gas temperature, 250° C.; sheath gas flow 11 L/min; capillary voltage, 3500 V; nozzle voltage, 500 V. The following parameters were used for data acquisition in multiple reaction monitoring (MRM) mode: dwell time, 50 ms; cell accelerator voltage, 4 V; fragmentor voltage, 90 V and collision energy, 16 V for SA and SA-d4; fragmentor voltage, 130 V and collision energy, 9 V for ABA-d6. The following MRM transitions were monitored: SA (m/z 137→93), SA-d4 (m/z 141→97) and ABA-d6 (m/z 269.1→159.1). Peak selection and integration of acquired MRM data files was done using QuanLynx v4.1 software (Waters, Milford, MA) or Quantitative Analysis (for QQQ) program in MassHunter software (Agilent). Analyte levels were calculated as previously indicated (Huot, 2017a).

Chromatin immunoprecipitation (ChIP): ChIP was performed as previously reported (Huang, 2009), with some modifications. Collected fresh leaf tissues were fixed (1% formaldehyde in 1× phosphate buffered saline [PBS]) by vacuum infiltration and incubated for 10-15 min to crosslink at room temperature. After quenching the remaining fixation solution with 125 mM glycine solution for 5 min, plant tissues were flash-frozen in liquid nitrogen and ground by mortar and pestle. Six hundred mg of ground powder were dissolved in 2 mL of nuclei isolation buffer and crude extracts were filtered with two layers of Miracloth (Millipore). To collect nuclei, the filtrate was centrifuged at 10,000×g at 4° C. for 5 min and the pellet was suspended in 75 μl of nuclei lysis buffer (50 mM Tris pH 8.0, 10 mM EDTA pH 8.0, 1% SDS). After 30 min incubation on ice, 625 μl of ChIP dilution buffer (16.7 mM Tris pH 8.0, 167 mM NaCl, 1.2 mM EDTA, 1.1% Trion X 100, 0.01% SDS) were added and the samples were sonicated for 1 min in the cold room using Sonic Dismembrator (Thermo Fisher) or 5-6 min using Bioruptor (Diagenode). After adding 200 μl of ChIP dilution buffer and 100 μl of 10% Triton X-100, samples were spun at full speed for 5 min to remove debris. For pre-clearing, samples were incubated with 25 μl of magnetic protein A or G beads (Thermo Fisher) for 2 h in the cold room. Twenty μl of samples were removed as 2% input samples. To capture the DNA/protein complex, antibodies (Table 3) were used for immunoprecipitation and samples were incubated (with rotation) overnight in the cold room using a tube rotator. After washing, DNA samples were recovered using elution buffer and incubated overnight at 65° C. to remove crosslinking. DNA samples were collected and purified using a QIAquick PCR Purification Kit (Qiagen). ChIP-qPCR was performed as described in the Gene expression analyses section. ChIP-qPCR primer sequences are listed in Table 2.

Nuclear cytoplasmic fractionation: Approximately 0.1-0.2 g of ground plant tissues (pre-frozen, stored at −80° C. for less than 1 week) were dissolved in nuclei isolation buffer (20 mM Tris-Cl pH 7.5, 25% Glycerol, 20 mM KCl, 2.5 mM MgCl₂, 2 mM EDTA, 250 mM Sucrose, 1× Protease inhibitor cocktail [Roche]) in ice (NPR1-YFP protein analysis) or at 23° C. or 28° C. (GBPL3 protein analysis). After removing debris by filtering with two layers of Miracloth (Millipore), collected extracts were centrifuged at 1000×g for 10 min at cold room or at 23° C. or 28° C. using temperature-controlled centrifuge. Supernatants were collected as the cytosolic fraction and pellets were suspended in nuclei washing buffer (nuclei isolation buffer supplemented with 0.1% Triton X-100) (Sigma-Aldrich) by gentle tapping and centrifuged at 1000×g for 10 min at 4° C. After washing twice, pellets were resuspended in nuclei isolation buffer and collected as nuclear fractions, which were further used for analysis.

Immunoblot: Ground plant tissues (0.2g/1 ml LDS buffer) or fractionated protein samples (1:1 v/v) were mixed with 2×LDS buffer (Genscript) in the presence or absence of 2-mercaptoethanol (Sigma-Aldrich) and boiled at 70° C. for 5 min. After removing debris by centrifugation, protein samples were resolved using SDS-PAGE (SurePAGE, Genscript) and transferred to PDVF membrane (Millipore) using a wet transfer system (Bio-Rad; transfer buffer from Thermo Scientific) for further analysis. Transferred blot was incubated in PBS-T (1×PBS, 0.05% Tween-20) supplemented with 5% non-fat dried milk for 1 h and relevant proteins were detected using specific antibodies. Chemiluminescence from blots was generated after adding Supersignal West dura or West femto substrate (Thermo Scientific) and detected by a ChemiDoc MP imaging system (Bio-Rad) or iBright CL 1500 (Thermo Scientific). Relative protein quantification was performed using iBright CL 1500 (Thermo Scientific) and FIJI/ImageJ software (win64 1.52i version). Experimental conditions for antibodies are in Table 3.

TABLE 3
Antibodies used.

| Antibody Name | Dilution Used | Purpose | Reference |
|---|---|---|---|
| Anti-GFP | 1:250 | ChIP | Cat. No. ab290, |
| | | | Abcam |
| Anti-GFP | 1:5000 | Western blotting | Cat. No. 632381, |
| | | (primary | Clontech, |
| | | antibody) | |
| Anti-myc | 1:200, 1:5000 | ChIP, Western | Cat. No. ab9106, |
| | | blotting (primary | Abcam |
| | | antibody) | |
| Anti-FLAG | 1:200 | ChIP | Cat. No. A00170, |
| | | | Genscript |
| Anti-FLAG | 1:2500 | Western blotting | Cat. No. A00187, |
| | | (primary | Genscript |
| | | antibody) | |
| Anti-MED6 | 1:100, 1:1000 | ChIP, Western | Agrisera, AS14 |
| | | blotting (primary | 2802 |
| | | antibody) | |
| Anti-RNA | 1:250 | ChIP | Cat. No. ab5131, |
| polymerase II | | | Abcam |
| Anti UGPase | 1:5000 | Western blotting | Cat. No. AS05 |
| | | (primary | 086, Agrisera |
| | | antibody) | |
| Anti-Rabbit-HRP | 1:5000 | Western blotting | Agrisera, AS09 |
| | | (secondary | 602 |
| | | antibody) | |
| Anti-Mouse-HRP | 1:2000-5000 | Western blotting | GE, NA931 |
| | | (secondary | |
| | | antibody) | |
| Anti-Mouse-HRP | 1:2000-5000 | Western blotting | Cell Signaling, |
| | | (secondary | 7076S |
| | | antibody) | |

Confocal laser scanning microscopy and image analysis of Arabidopsis and tobacco cells: Images were acquired with the Zeiss confocal laser scanning microscopy 880 system and Zen black software (Carl Zeiss). Pre-treated leaves of 4-5 weeks old plants (35S::eGFP-GBPL3) were imaged with an inverted Zeiss 880 single point scanning confocal attached to a fully motorized Zeiss Axio Observer microscope base, with Marzhauser linearly encoded stage and a 63× NA 1.4 oil plan apochromatic oil immersion objective lens. Images were acquired by frame (line) scanning unidirectionally at 0.24 microseconds using the galvanometer-based imaging mode, with a voxel size of 0.22 μm×0.22 μm×1 μm and an area size of 224.92 μm×224.92 μm×1 μm in Zeiss Zen Black Acquisition software and saved as CZI files. eGFP and chlorophyll was excited at 488 nm excitation laser from argon laser source and detected at 490-526 or 653-683 nm, respectively. Equal acquisition conditions (e.g., excitation laser source intensity, range of acquired emission light range, and exposure condition) were used for every image in each experiment. To maintain appropriate temperature during experiments, a portable temperature chamber and temperature-controlled specimen chamber of confocal microscope were used. To analyze images, FIJI/ImageJ software (win64 1.52i version) was used.

Prediction of intrinsically disordered region of AtMED15: The AtMED15 protein (Atlg15780) disordered region was calculated through Predictor of Natural Disordered Regions online tool (www.pondr.com). The AtMED15 amino acid sequence was obtained from The Arabidopsis Information Resource (TAIR, www.arabidopsis.org).

Statistical analysis: Experimental sample size was chosen based on previously published literature to be sufficient for statistical analyses. Three to four plants (biological replicates) per genotype per treatment were analyzed per individual experiment. Three or more independent experiments were performed for all assays, unless specified otherwise. The following statistical analyses were employed: (1) Student's t-test with Bonferroni test for significance was used for pairwise comparisons; (2) one-way analysis of variance (ANOVA) with Bartlett's test for significance was used for multi-sample experiments with one variable; and (3) two-way analysis of variance ANOVA followed by Tukey's honest significant difference (HSD) test was employed for multi-variable analyses. Statistical tests are described in the figure legends. Bar graphs and dot plots were generated by GraphPad Prism 9 and show the means±S.D. (or +S.E.M.) and individual data points.

### Initial Investigations

### Example 2: Role of SA Receptor and Biosynthesis Genes

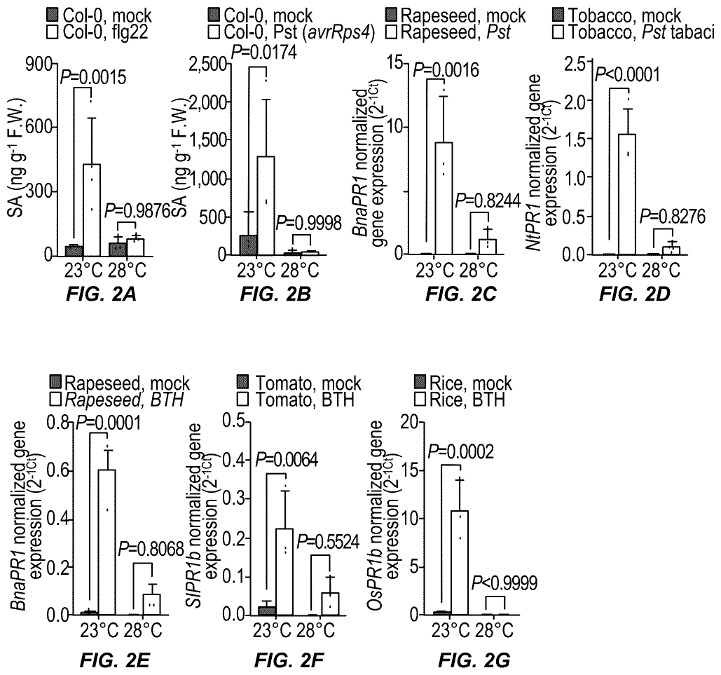

Because expression of ICS1 is a crucial step in SA production and is downregulated at ET16, the inventors tested whether downregulated IC′S1 (data not shown, see FIG. 1B of U.S. Provisional Application No. 63/523,390) is the rate-limiting step controlling ET-mediated SA suppression. Surprisingly, although constitutive ICS1 expression from the 35S CaMV promoter resulted in constitutive accumulation of SA at 23° C., as expected, it failed to restore SA production at 28° C. and the ICS1-overexpressing plants showed increased disease susceptibility at 28° C., just like wild-type Col-0 plants (data not shown, see FIG. 1C; Extended Data (ED) FIGS. 2G-2H of U.S. Provisional Application No. 63/523,390). SA accumulation is also regulated by the SA receptors (NPR proteins); however, constitutive activation of NPR1 using npr 1^{S11D/S15D} phosphomimetic lines failed to restore SA accumulation and these plants exhibited hyper-susceptibility to Pst DC3000 infection at 28° C. (data not shown, see FIG. 1D; ED FIGS. 2I-2J of U.S. Provisional Application No. 63/523,390). Finally, removal of antagonistic SA receptors NPR3 and NPR4 using the npr3 npr4 double mutant also could not counter ET-suppression of SA immunity (data not shown, see ED FIGS. 2K-2M of U.S. Provisional Application No. 63/523,390).

Overall, these results highlighted the challenges to identify the primary, rate-limiting step in the SA pathway that is impacted by heat waves based on established plant thermosensing and SA biosynthesis/receptor paradigms.

Initial evidence of CBP60g as a primary temperature-vulnerable step: The failure of constitutive ICS1 expression and NPR1 activation to restore SA production at ET (data not shown, see FIGS. 1C-1D of U.S. Provisional Application No. 63/523,390) led the inventors to pursue a different strategy. Specifically, we performed RNA-sequencing of Pst DC3000-infected Col-0 plants at normal and ET (data not shown). We found that, in addition to ICS1, pathogen-induction of various SA-associated defense regulators was suppressed at 28° C. (Table 4, Cluster A), including EDS1, PAD4 and WRKY75 (data not shown, see FIGS. 1E-1G of U.S. Provisional Application No. 63/523,390), whereas the SA catabolic gene BSMT1 was upregulated at 28° C. (data not shown, see FIG. 1H of U.S. Provisional Application No. 63/523,390). ET-downregulated Cluster A genes included CBP60g (data not shown, see FIG. 1I of U.S. Provisional Application No. 63/523,390) and SARD1, which encode two functionally redundant ICS1 promoter-binding transcription factors required for SA production. Monitoring a GUS reporter gene fused to the CBP60g promoter (Col-0/pCBP60g::GUS) also detected decreased transcript levels at 28° C. (data not shown, see FIG. 2A of U.S. Provisional Application No. 63/523,390), indicating that ET affects CBP60g expression mainly through transcription initiation or preinitiation at core promoter regions, instead of via transcription elongation or termination at gene body or downstream events. Further examination revealed that a large number of CBP60g SARD1 target genes were suppressed at 28° C. (data not shown, see FIG. 1J of U.S. Provisional Application No. 63/523,390), including many known crucial regulators of basal and systemic immunity (data not shown, see FIG. 1K of U.S. Provisional Application No. 63/523,390), which raises the intriguing possibility that CBP60g expression may be the primary target in ET-mediated suppression of SA production.

TABLE 4
Transcriptome clustering and GO-enrichment of Pst DC3000-regulated genes in
Arabidopsis plants at 23° C. (ambient) vs. 28° C. (ET)

| | | | | | CBP60g/ |
|---|---|---|---|---|---|
| | | | | | SARD1 |
| Pathogen | Temperature | | Number | | target |
| effect | effect | Cluster | of genes | Top 5 GO terms | genes |
| Pst DC3000- | Down at ET | A | 717 | Defense response | 140 |
| induced | | | | Systemic acquired | |
| | | | | resistance | |
| | | | | Response to fungus | |
| | | | | Response to bacterium | |
| | | | | Response to salicylic acid | |
| | Similar at ET | B | 1236 | Response to jasmonic acid | 126 |
| | | | | Response to wounding | |
| | | | | Negative regulation of | |
| | | | | nucleic acid-templated | |
| | | | | transcription | |
| | | | | Regulation of jasmonic | |
| | | | | acid mediated signaling | |
| | | | | pathway | |
| | | | | Jasmonic acid | |
| | | | | biosynthetic process | |
| | Up at ET | C | 619 | Response to jasmonic acid | 22 |
| | | | | Response to wounding | |
| | | | | Mesponse to high light | |
| | | | | intensity | |
| | | | | Metabolic process | |
| | | | | response to karrikin | |
| Pst DC3000- | Down at ET | D | 845 | Response to chitin | 83 |
| suppressed | | | | Protein phosphorylation | |
| | | | | Response to light stimulus | |
| | | | | Response to cold | |
| | | | | Purine nucleobase | |
| | | | | transport | |
| | Similar at ET | E | 1501 | Circadian rhythm | 66 |
| | | | | Response to auxin | |
| | | | | Regulation of | |
| | | | | Transcription, DNA- | |
| | | | | templated | |
| | | | | Xylan catabolic process | |
| | | | | Auxin-activated signaling | |
| | | | | pathway | |
| | Up at ET | F | 402 | Cold acclimation | 3 |
| | | | | Response to water | |
| | | | | Transcription, DNA- | |
| | | | | templated | |
| | | | | Response to water | |
| | | | | Deprivation | |
| | | | | response to abscisic acid | |

Differentially expressed genes (DEGs) were categorized as either pathogen-induced or pathogen-suppressed. These DEGs were further classified as downregulated at elevated temperature (“Down at ET”), similar between the two temperatures (“Similar) and upregulated at elevated temperature (“Up at ET”). The number of genes and top 5 Gene Ontology (GO) terms based on DAVID are indicated, along with how many of these genes are present in the CBP60g homolog SARD1 ChIP-Seq dataset.

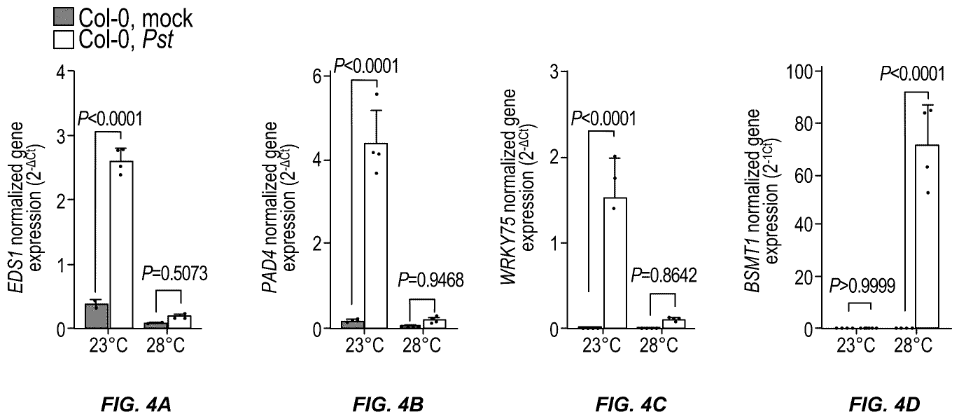

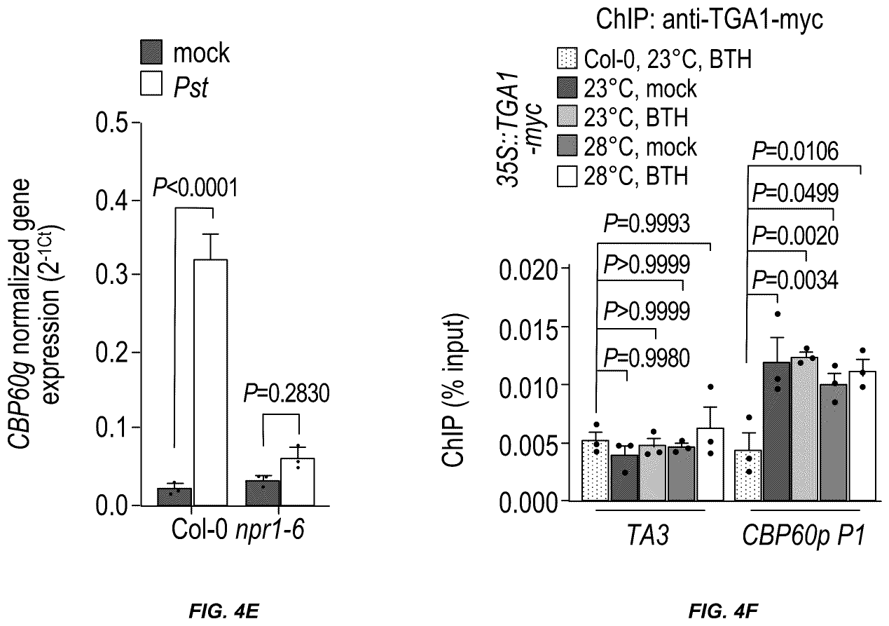

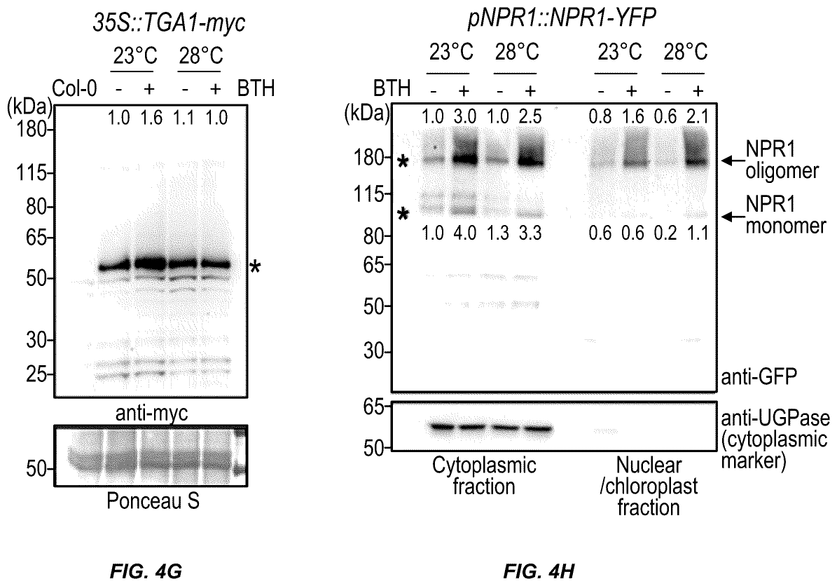

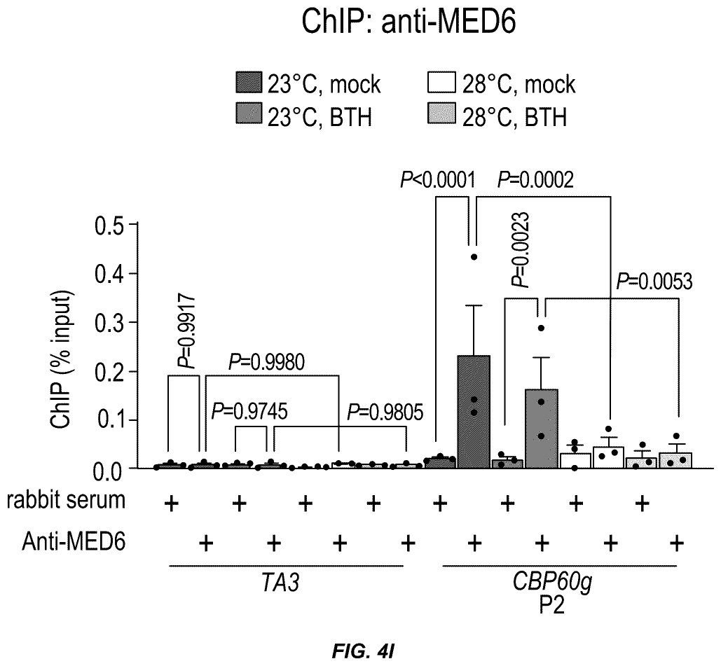

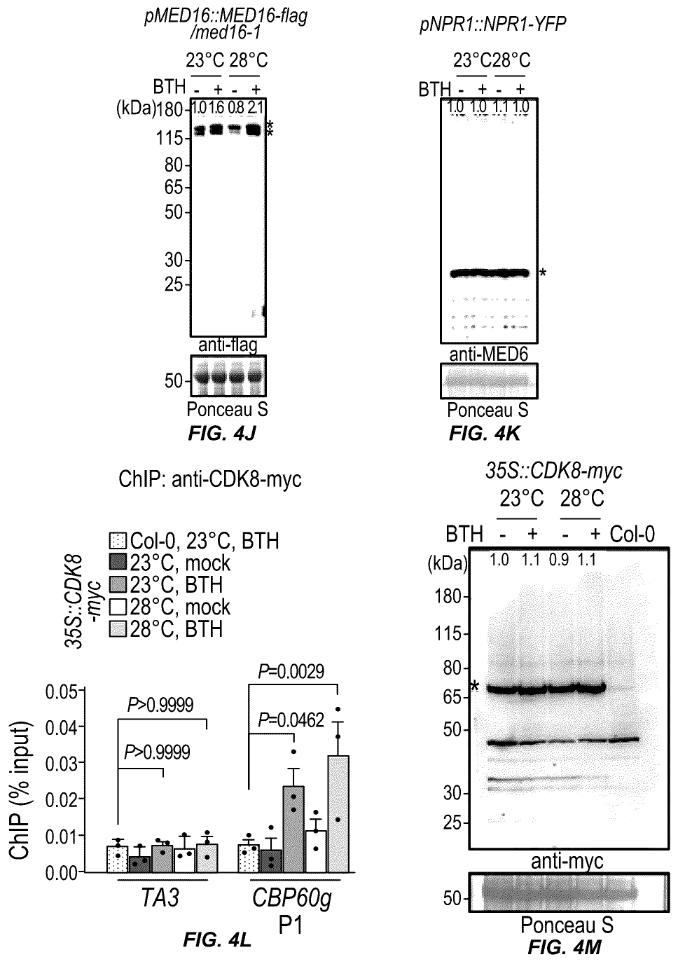

Temperature-sensitivity of GBPL3 phase separation and recruiting: The current SA signaling model suggests that NPR receptor proteins act through TGA transcription factors, which regulate CBP60g gene expression and SA biosynthesis. To gain a mechanistic understanding of how ET affects CBP60g transcription, we investigated the impact of ET on known CBP60g regulators. The SA receptor NPR1 is required for induced CBP60g gene expression (data not shown, see ED FIG. 3A of U.S. Provisional Application No. 63/523,390). Although CBP60g transcripts at ET decreased at 28° C. following treatment of plants with BTH (data not shown, see FIG. 2A of U.S. Provisional Application No. 63/523,390), levels of NPR1 recruitment to the CBP60g promoter were similar at both 23° C. and 28° C. after chromatin immunoprecipitation (ChIP) (data not shown, see FIG. 2B of U.S. Provisional Application No. 63/523,390). Consistent with this result, NPR1 monomerization, which is associated with NPR1 function, was similar at both temperatures (data not shown, see ED FIG. 3B of U.S. Provisional Application No. 63/523,390). We also found that constitutive TGA1 expression did not restore SA levels at ET and that 35S::TGA1 plants still exhibited temperature-sensitive disease susceptibility (data not shown, see ED FIGS. 2N-2P of U.S. Provisional Application No. 63/523,390). In agreement, binding of the TGA1 transcription factor to the CBP60g promoter and total TGA1 protein levels were not affected at 28° C. (data not shown, see FIG. 2C; ED FIG. 4A of U.S. Provisional Application No. 63/523,390). Taken together, these results pointed to an NPR1/TGA1-independent suppression mechanism for CBP60g transcription and SA production at ET.

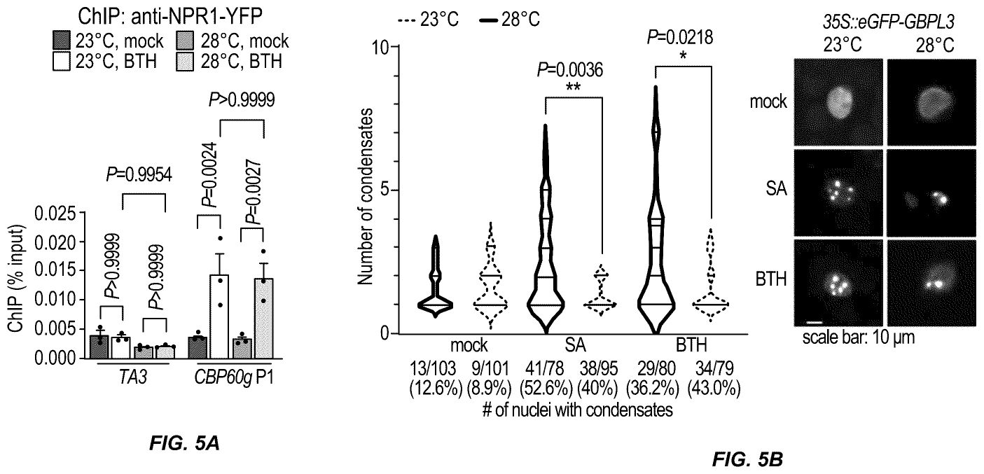

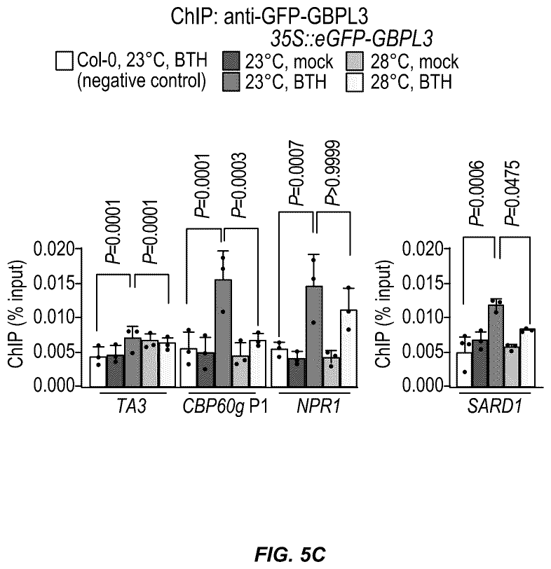

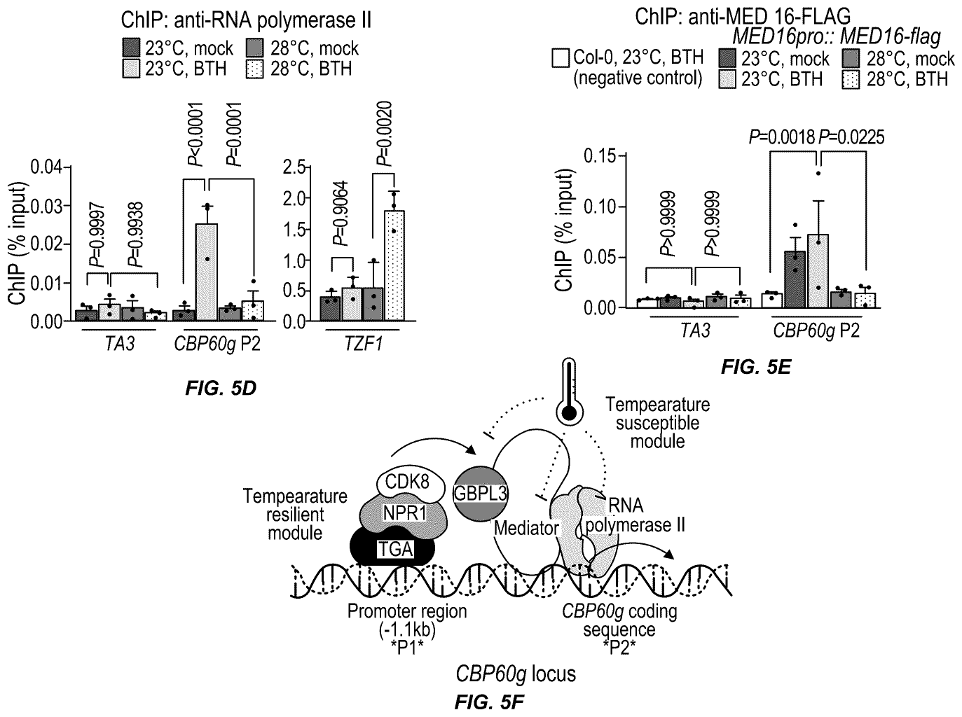

A recent report identified Guanylate-Binding Protein-Like 3 (GBPL3) as an important positive regulator of SA signaling. CBP60g gene expression is found to be upregulated in GBPL3 overexpression plants and we found in this study that GBPL3 is required for CBP60g gene expression in response to SA (data not shown, see ED FIG. 5 of U.S. Provisional Application No. 63/523,390). GBPL3 has been proposed to act on gene promoters via phase-separated biomolecular condensates together with Mediator and RNA polymerase II (Pol II) 13. Intriguingly, like the thermosensor ELF3 involved in thermomorphogenesis, GBPL3 has an intrinsically disordered domain (IDR), which mediates formation of GBPL3 biomolecular condensates. Therefore, we tested the hypothesis that ET may negatively affect GBPL3 condensate formation and/or binding to the CBP60g promoter. Indeed, we observed that there were significantly reduced GBPL3 condensates per nucleus at 28° C. compared to 23° C. (data not shown, see FIG. 2D of U.S. Provisional Application No. 63/523,390). ChIP-qPCR experiments revealed that GBPL3 binding to the CBP60g promoter was markedly reduced at 28° C., compared with that at 23° C., in plants treated with BTH (data not shown, see FIG. 2E of U.S. Provisional Application No. 63/523,390), even though GBPL3 protein levels remained similar at both temperatures (data not shown, see ED FIG. 4B of U.S. Provisional Application No. 63/523,390). In contrast, GBPL3 recruitment to the NPR1 promoter was not compromised (data not shown, see FIG. 2E of U.S. Provisional Application No. 63/523,390), consistent with temperature resilient NPR 1 transcript levels. This result suggests that ET-mediated suppression of GBPL3 recruitment occurs selectively at certain loci, such as CBP60g, but not globally at all target sites of GBPL3. Consistent with this result, we observed that ET did not decrease the number of nuclei that contained GBPL3 condensates, despite significantly reduced GBPL3 condensates per nucleus at 28° C. compared to 23° C. (data not shown, see FIG. 2D of U.S. Provisional Application No. 63/523,390). Collectively, our data indicate that ET selectively affects GBPL3 recruitment to the CBP60g promoter in association with altered GBPL3 phase separation.

Next, we investigated if the altered GBPL3 condensate formation and reduced GBPL3 protein binding to the CBP60g promoter at ET is linked to an impairment in the recruitment of Pol II and Mediator subunits to the CBP60g promoter. As shown in (data not shown, see FIG. 2F of U.S. Provisional Application No. 63/523,390), ET suppressed BTH-induced Pol II association with the CBP60g promoter, but not with the promoter of a control gene TZF1, which is highly induced by BTH at ET16, consistent with selective suppression of the SA pathway by ET. Furthermore, there was a significant reduction in CBP60g promoter binding at 28° C. by MED16, a Mediator tail subunit previously shown to be associated with SA gene expression (data not shown, see FIG. 2G of U.S. Provisional Application No. 63/523,390). Binding of a Mediator head subunit, MED6, to the CBP60g promoter was also significantly reduced at 28° C. compared to that at 23° C. (data not shown, see FIG. 2H of U.S. Provisional Application No. 63/523,390). Differential Mediator subunit recruitment was not due to different protein abundance, since MED16 and MED6 protein levels remained unchanged at 23° C. and 28° C. (data not shown, see ED FIGS. 4C-4D of U.S. Provisional Application No. 63/523,390). Interestingly, not all components of the Mediator are affected at ET, as the level and binding of CDK8, a subunit of the Mediator kinase module that interacts with NPR1 to regulate SA signaling, were similar at 23° C. and 28° C. (data not shown, see FIG. 2I; ED FIG. 4E of U.S. Provisional Application No. 63/523,390). These results give insight into the effect of ET on CBP60g promoter-associated transcriptional machinery, indicating that that ET selectively affects the recruitment of GBPL3 and several SA pathway-relevant subunits of the Mediator complex to the CBP60g gene promoter, independently of the NPR1-TGA1-CDK8 module (data not shown, see FIG. 2J of U.S. Provisional Application No. 63/523,390).

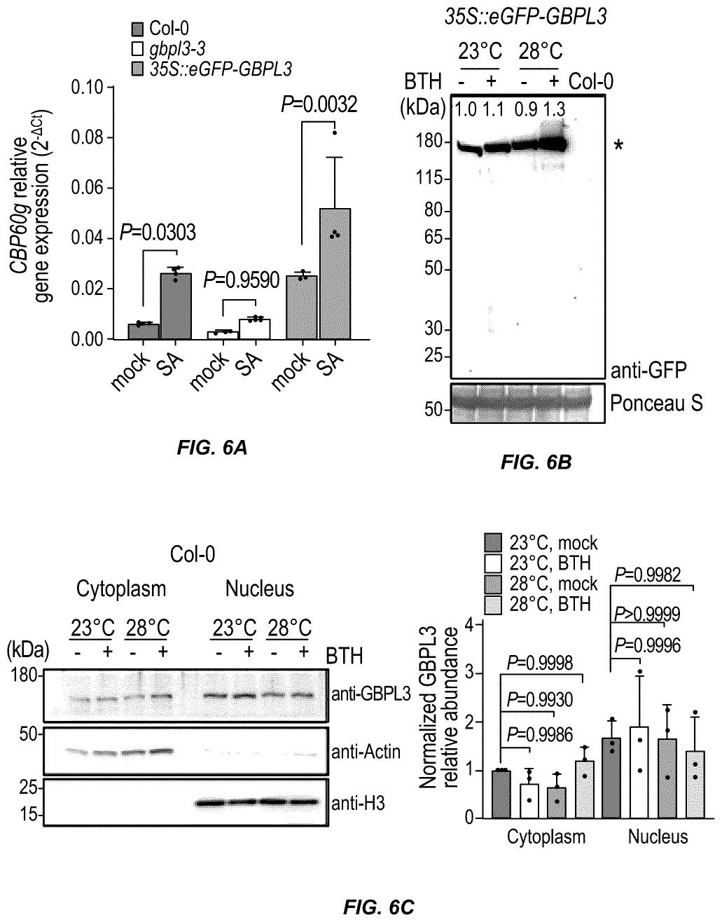

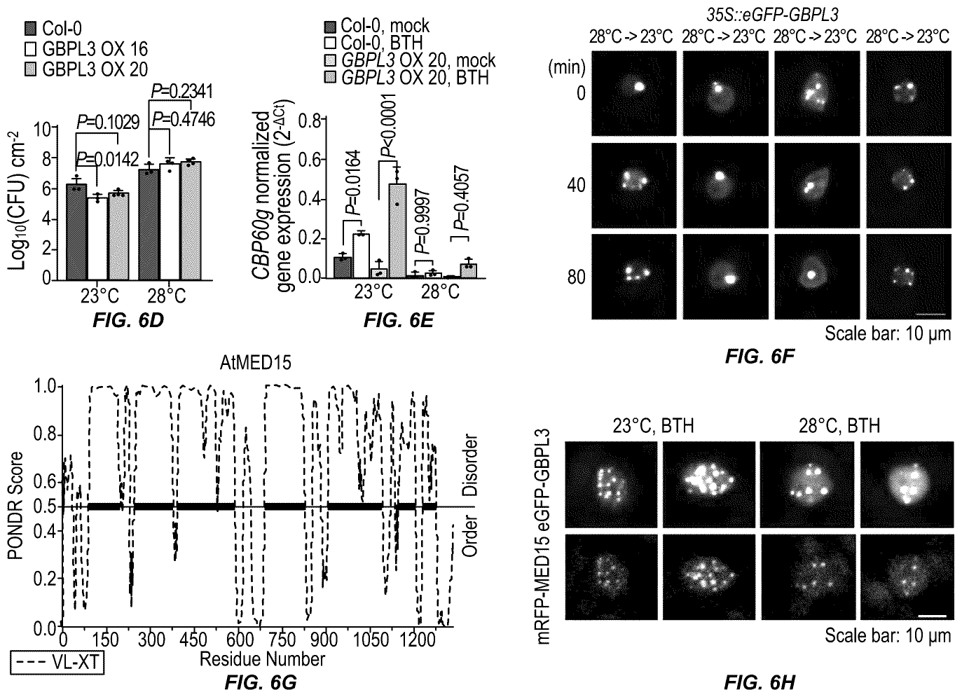

CBP60g is a rate-limiting step of SA-mediated immunity at ET: Having identified CBP60g transcription as the primary ET-sensitive step in the SA pathway, we next asked if reduced CBP60g expression is a rate-limiting step during ET-mediated suppression of SA immunity and, if so, whether restoring CBP60g expression would be sufficient to render SA biosynthesis resilient to heat wave. We found that, unlike expression of the activated SA receptor NPR1 or the SA biosynthetic gene ICS1 (data not shown, see FIGS. 1C-1D; FIGS. 2G-2J of U.S. Provisional Application No. 63/523,390), 35S::C′BP60g lines maintained SA production and basal immunity to Pst DC3000 infection at 28° C., in contrast to wild-type Col-0 plants (data not shown, see ED FIG. 3A; ED FIG. 6A of U.S. Provisional Application No. 63/523,390). Furthermore, 35S::CBP60g showed substantial insensitivity to ET-induced disease hypersusceptibility to Pst DC3000 infection at 28° C. (data not shown, see ED FIGS. 3B-3C; ED FIGS. 6B-6C of U.S. Provisional Application No. 63/523,390). Consistent with these immune phenotypes, 35S::CBP60g plants had restored expression of the CBP60g target genes IC′S1, EDS1 and PAD4 at 28° C. in response to Pst DC3000 infection (data not shown, see ED FIGS. 3D-3F of U.S. Provisional Application No. 63/523,390).

In addition to the virulent pathogen Pst DC3000, we found that the ET-resilient phenotype of 35S::CBP60g lines extends to infection by avirulent pathogens Pst DC3000 (avrPphB) and Pst DC3000 (avrRps4) (data not shown, see ED FIGS. 3G-3H of U.S. Provisional Application No. 63/523,390), which activate ETI through either CC- or TIR-containing NLRs, respectively. Because ETI is widely used in crop fields to guard crops against pathogens and insects, these results suggest potentially broad applications of restoring CBP60g expression to counter ET-suppression of not only basal immunity to virulent pathogens, but also ETI against pathogen infection.

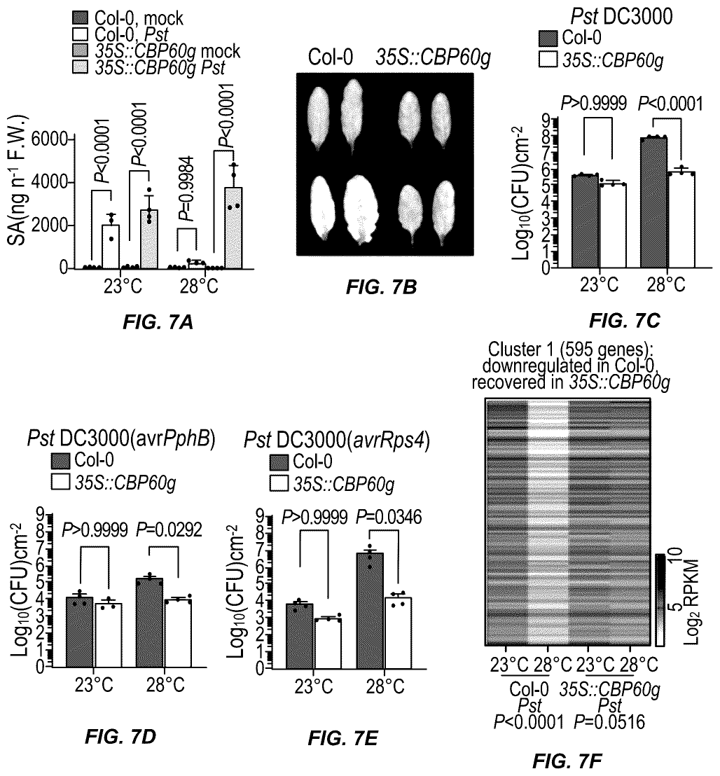

Notably, the ability of 35S::CBP60g plants to restore SA production and immunity appears to be unique among known regulators of the SA pathway. Constitutively expressing many other ET-downregulated positive SA regulators, including ICS1, TGA1, EDS1, PAD4 and WRKY7, all failed to restore SA production or basal immunity to Pst DC3000 infection (data not shown, see FIG. 1C; ED FIGS. 2G-2H and 2N-2P; ED FIGS. 7A-7C of U.S. Provisional Application No. 63/523,390). Similarly, mutating the ET-upregulated negative regulators, such as SA catabolic gene BSMT1 (data not shown, see ED FIG. 7D of U.S. Provisional Application No. 63/523,390), or SA transcriptional repressor genes (AMTA2/3, also failed to restore SA levels and disease resistance at ET (data not shown, see ED FIG. 7E of U.S. Provisional Application No. 63/523,390). Together with the inability of activated SA receptor NPR1 or the SA biosynthetic gene ICS1 to restore SA production under ET (data not shown, see FIGS. 1C-D; ED FIGS. 2G-2J of U.S. Provisional Application No. 63/523,390), these results illustrate that CBP60g is a unique SA pathway regulator, the level of which becomes rate-limiting for controlling SA production and immunity at ET.

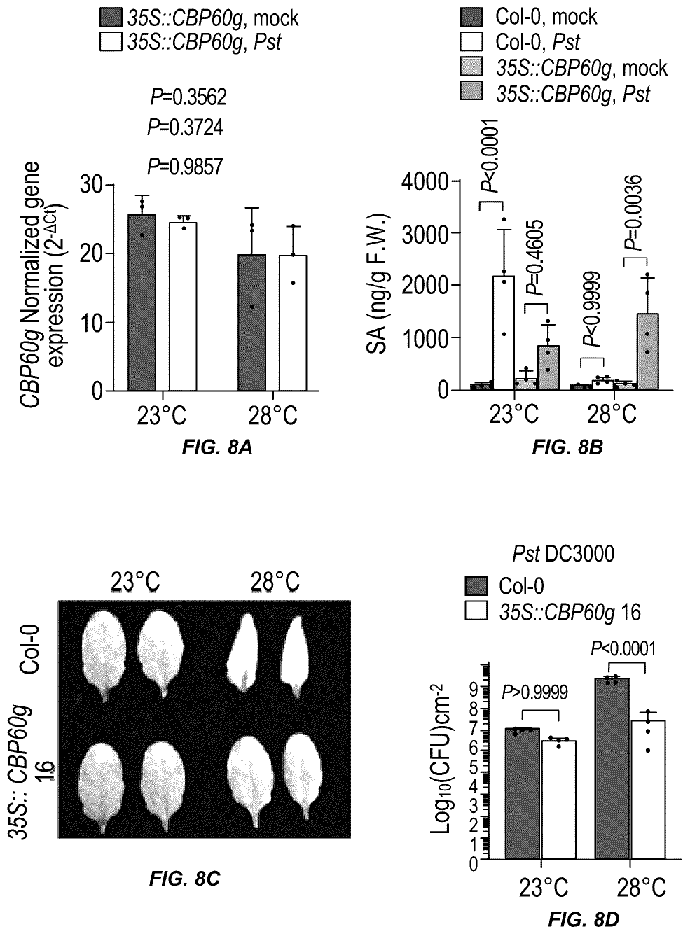

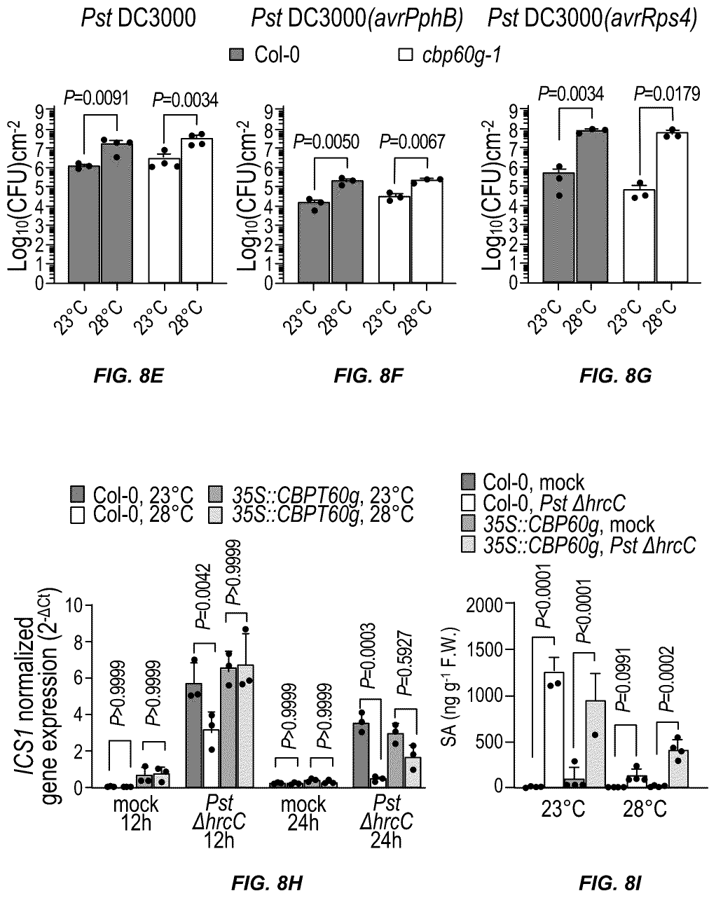

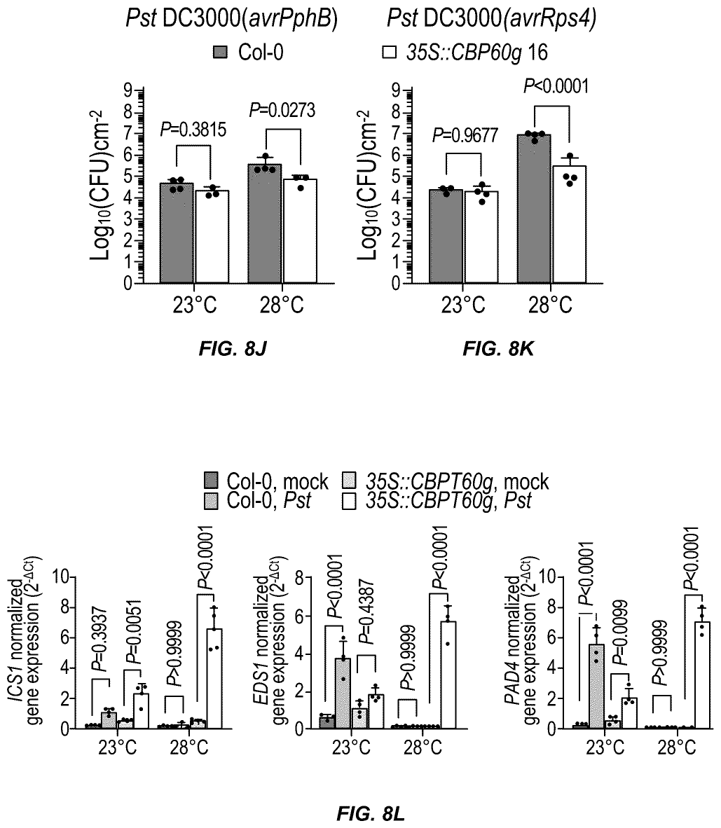

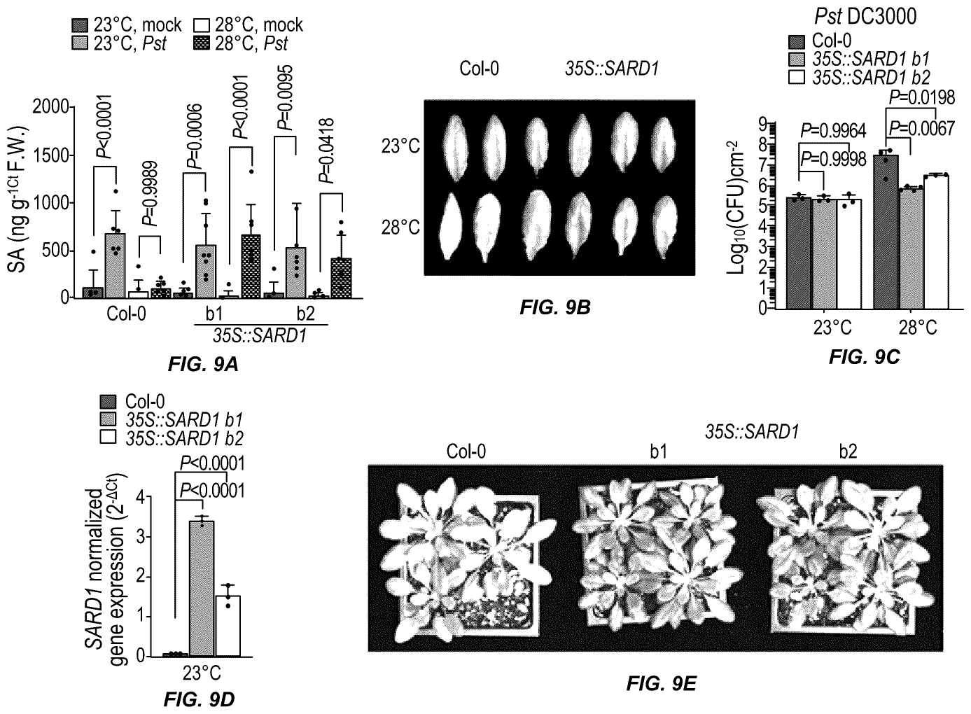

Optimization of growth-defense trade-off in CBP60g expression: A common issue with increasing the expression level of SA regulators is inhibition of plant growth and reproduction due to growth-defense tradeoff. This is illustrated with 35S::ICS1 plants, which have elevated SA levels at ambient temperature (data not shown, see FIG. 1C of U.S. Provisional Application No. 63/523,390) and were characteristically dwarf (data not shown, see ED FIGS. 8A-8B of U.S. Provisional Application No. 63/523,390). Interestingly, the final vegetative biomass of 35S::CBP60g was not as adversely affected as 35S::ICS1 (data not shown, see ED FIGS. 8A-8B of U.S. Provisional Application No. 63/523,390), consistent with low basal SA levels in 35S::CBP60g plants (data not shown, see FIG. 3A; ED FIG. 6A of U.S. Provisional Application No. 63/523,390). Nevertheless, 35S::CBP60g plants showed a significant delay in flowering (data not shown, see ED FIG. 8C of U.S. Provisional Application No. 63/523,390). To minimize this developmental trade-off, we expressed CBP60g using the uOREs_{TBF1} strategy (data not shown, see ED FIG. 9 of U.S. Provisional Application No. 63/523,390), which allows tightly controlled protein translation in response to pathogen infection. As shown in (data not shown, see FIGS. 4A-4C of U.S. Provisional Application No. 63/523,390), 35S::uOREs_{TBF1}-CBP60g plants maintained basal Pst DC3000 resistance and SA production at 28° C. These plants also maintained substantial ETI against Pst DC3000 (avrPphB) and Pst DC3000 (avrRps4) at ET (data not shown, see FIG. 4D of U.S. Provisional Application No. 63/523,390). Notably, 35S::uORFs_{TBF1}-CBP60g plants did not exhibit obvious growth defects and had a normal flowering time (data not shown, see FIG. 4E of U.S. Provisional Application No. 63/523,390), demonstrating the promise of leveraging calibrated CBP60g expression to preserve plant immunity without significantly detrimental growth/developmental impacts.

### Updated Investigations

### Example 3: Temperature-Vulnerability of the SA Pathway

The model plant Arabidopsis thaliana accession Col-0 becomes hypersusceptible to the virulent pathogen Pseudomonas syringae pv. tomato (Pst) DC3000 during a short period of heat wave (Huot, 2017a), (FIG. 1A). Elevated temperature also suppressed the expression of ISOCHORISMATE SYNTHASE 1 (ICS1; Huot, 2017a), (FIG. 1B), a key SA biosynthetic gene (Wildermuth, 2001), leading to reduced SA accumulation at 28° C. compared to 23° C. (FIG. 1C). Although elevated temperature does not affect MAP kinase activation during early stages of pattern-triggered immunity (PTI) in response to bacterial flagellin-derived flg22 peptide (Cheng, 2013), downstream SA accumulation is significantly reduced (FIG. 2A). Furthermore, consistent with previous studies showing suppressed effector-triggered immunity (ETI) at elevated temperature (Cheng, 2013; Samaradivakara, 2022; Wang, 2009; Zhu, 2010), it was discovered here that SA accumulation in Arabidopsis Col-0 plants is suppressed at 28° C. after infection with an ETI-activating P. syringae strain (FIG. 2B). Finally, elevated temperature downregulated the expression of SA-response genes in both dicot (rapeseed, tobacco and tomato) and monocot (rice) crop plants, after pathogen infection and/or pathogen-independent elicitation with benzothiadiazole (BTH, a synthetic SA analog) (FIG. 2C-2G). Together, these results suggest that the temperature-vulnerability of the SA pathway is likely a common feature in plants and has pervasive effects on basal immunity, PTI and ETI.

### Example 4: Independent of phyB/ELF3 Thermosensors

Recent studies showed that phyB (Jung, 2016; Legris, 2016) and ELF3 (Jung, 2020) regulate thermoresponsive plant growth and development. To determine whether heat wave suppression of SA production also occurs via these thermosensing mechanisms, we tested constitutively activated phyB (35S::PHYB^{Y276H}; Jung, 2016) or ELF3 thermosensor (BdELF3-OE; Jung, 2020) lines that do not exhibit thermos-responsive growth. However, these plants remained temperature-sensitive in pathogen-induced SA accumulation and displayed increased bacterial susceptibility at 28° C. (FIGS. 3A-3F). These results indicate that SA suppression at elevated temperature is independent of phyB or ELF3 thermosensing mechanisms. This agrees with our previous study showing that neither activated phyB nor quadruple mutants in PHYTOCHROME-INTERACTING FACTORS (pif) conferred temperature-resilient basal immunity to Pst DC3000 infection during a simulated heat wave (Huot; 2017a).

### Example 5: Beyond SA Biosynthesis/Receptor Genes

Because ICS1 expression is crucial for SA production (Wildermuth, 2001) and is downregulated at elevated temperature (Huot; 2017a), we next tested whether downregulated ICS1 (FIG. 1B) is the rate-limiting step controlling heat wave-mediated SA suppression. Surprisingly, although constitutive ICS1 expression from the 35S Cauliflower mosaic virus (CaMV) promoter resulted in constitutive SA accumulation at 23° C. as expected, it failed to restore pathogen induced SA at 28° C. and the ICS1-overexpressing plants showed compromised basal immunity at 28° C., just like wild-type Col-0 plants (FIG. 1D; FIGS. 3G-3H). SA accumulation is also regulated by the SA receptors (NPR proteins) (Peng, 2021; Ding, 2018); however, constitutive NPR1 activation using npr 1^{S11D/S15D} phosphomimetic lines (Saleh, 2015) failed to restore SA accumulation and these plants exhibited hypersusceptibility to Pst DC3000 at 28° C. (FIG. 1E; FIGS. 3I-3J). Finally, removal of antagonistic SA receptors NPR3 and NPR4 using the npr3 npr4 mutant (Ding, 2018) also could not counter suppression of SA immunity at elevated temperature (FIGS. 3K-3M). Overall, these results highlighted the challenges to identify the primary, rate-limiting step in the SA pathway that is impacted by heat waves based on well-established plant thermosensing (Jung, 2016; Legris, 2016; Jung 2020) and SA biosynthesis/receptor (Peng, 2021; Wildermuth, 2001; Ding, 2018; Saleh, 2015; Zhou, 2020) paradigms.

### Example 6: Impact on CBP60g/SARD1 Expression

The failure of constitutive ICS1 expression and NPR1 receptor activation to restore SA production at elevated temperature (FIGS. 1D-1E) led to a different strategy. Specifically, RNA-sequencing of Pst DC3000-infected Col-0 plants at normal and elevated temperatures was performed. We found that, in addition to ICS1, pathogen-induction of various SA-associated defense regulators was suppressed at 28° C. (Table 4, Cluster A), including EDS1, PAD4 and WRKY75 (FIGS. 4A-4C), whereas the SA catabolic gene BSMT1 was upregulated at 28° C. (FIG. 4D). Elevated temperature-downregulated Cluster A genes included CBP60g (FIG. 1F) and SARD1 (data not shown), which encode functionally redundant ICS/promoter-binding transcription factors required for SA production (Wang, 2009a; Zhang, 2010; Sun, 2015). Monitoring a GUS reporter fused to the CBP60g promoter also detected decreased transcript levels at 28° C. (FIG. 1G), indicating that elevated temperature affects CBP60g expression mainly through transcription. Further examination revealed that numerous CBP60g/SARD1 target genes (Sun, 2015) were suppressed at 28° C. (FIG. 1H), including many known crucial regulators of basal and systemic immunity (FIG. 1I), raising the intriguing possibility that CBP60g SARD1 expression may be the primary target in SA suppression at elevated temperature.

### Example 7: Thermosensitive GDACs and GBPL3 Binding

To mechanistically understand how elevated temperature affects CBP60g transcription, we investigated the impact of elevated temperature on known CBP60g regulators. The current SA signaling model suggests that NPR receptors interact with TGA transcription factors (Peng, 2021; Ding, 2018; Zhou, 2020), which regulate CBP60g gene expression (FIG. 4E) and SA biosynthesis. However, it was discovered here that constitutive TGA1 expression did not restore SA levels at elevated temperature and that 35S::TGA1 plants still exhibited temperature-sensitive basal immunity to Pst DC3000 (FIGS. 3N-3P). In agreement, TGA1 binding to the CBP60g promoter and total TGA1 protein levels were not affected at 28° C. (FIGS. 4F-4G). Similarly, NPR 1 recruitment to the CBP60g promoter was similar at 23° C. and 28° C. after chromatin immunoprecipitation (ChIP) (FIG. 5A). Consistent with this result, NPR1 monomerization, which is associated with NPR1 function (Mou, 2003), was similar at both temperatures (FIG. 4H). Altogether, these results pointed to an NPR1/TGA1-independent mechanism for suppressing CBP60g transcription and SA production at elevated temperature.

A recent report identified GBPL3 as an important positive regulator of SA signaling and immunity¹¹. The inventors have presently found that GBPL3 is required for CBP60g gene expression in response to SA (FIG. 6A). GBPL3 has been proposed to act on promoters via phase-separated biomolecular condensates together with Mediator and RNA polymerase (Pol) II (Huang, 2021). Intriguingly, like the thermosensor ELF3, which contains an intrinsically disordered domain (IDR) involved in condensate formation and temperature-sensing (Jung, 2020), GBPL3 also has an IDR, which mediates intranuclear GDAC formation (Huang, 2021). Therefore, the inventors tested whether elevated temperature negatively affects GDAC formation and/or GBPL3 recruitment to the CBP60g promoter necessary for CBP60g transcription. Indeed, the number of GDACs per nucleus was significantly reduced at 28° C. compared to 23° C. (FIG. 5B). ChIP-qPCR experiments revealed that GBPL3 binding to the CBP60g promoter and its functionally redundant paralog SARD1 were markedly reduced at 28° C. in BTH-treated plants (FIG. 5C), even though total GBPL3 protein levels remained similar at both temperatures (FIGS. 6B-6C). Consistent with the observation that the temperature effect is not at the level of GBPL3 expression, GBPL3 overexpression did not restore CBP60g expression (FIGS. 6D-6E). Interestingly, time-lapse imaging revealed that GDACs reversibly appeared at 23° C. or disappeared at 28° C. in response to temperature shifts, indicating that their formation and dissolution are temporally dynamic (FIG. 6F). Furthermore, MED15, another component of the GDAC (Huang, 2021) that contains multiple IDRs (FIG. 6G) also showed temperature-sensitivity (FIG. 6H). GBPL3 and MED15 were co-localized in individual GDACs, as observed previously (Huang, 2021), and they either co-appeared or co-disappeared in response to elevated temperature.

### Example 8: GBPL3 Specificity on CBP60g/SARD1 Loci

The inventors found that elevated temperature-mediated suppression of GBPL3 recruitment occurs selectively at certain loci, but not at all GBPL3 target sites. For example, elevated temperature suppressed GBPL3 recruitment to CBP60g and SARD1, but not to NPR1 NPR1 (FIG. 5C), which is consistent with temperature resilient NPR1 transcript levels¹⁵. Interestingly, it was discovered here that, despite significantly reduced GDACs per nucleus, elevated temperature did not decrease the number of nuclei that contained GDACs (FIG. 5B). Collectively, the findings here indicate that there appear to be two GDAC subpopulations in vivo. One subpopulation is sensitive to 28° C. (associated with GBPL3 recruitment to the CBP60g promoter) and the other insensitive to 28° C. (associated with GBPL3 recruitment to the NPR1 promoter).

Next, it was investigated if altered GBPL3 condensate formation and reduced GBPL3 binding to the CBP60g promoter at 28° C. is linked to impaired recruitment of Pol II and Mediator subunits. As shown in FIG. 5D, elevated temperature suppressed BTH-induced Pol II association with the CBP60g promoter, but not with the promoter of a control gene TZF1, which is highly induced by BTH at elevated temperature (Huot, 2017a). Furthermore, elevated temperature significantly reduced CBP60g promoter binding by MED16, a Mediator tail subunit associated with SA gene expression (Zhang, 2012) (FIG. 5E). Binding of a Mediator head subunit, MED6, to the CBP60g promoter was also significantly reduced at 28° C. compared to 23° C. (FIG. 4I). Differential Mediator subunit recruitment was not due to different protein abundance, since MED16 and MED6 protein levels remained unchanged at 23° C. and 28° C. (FIGS. 4J-4K). Interestingly, not all Mediator components are affected at elevated temperature, as the level and binding of CDK8, a Mediator kinase module subunit that interacts with NPR1 to regulate SA signaling (Chen, 2019), were similar at 23° C. and 28° C. (FIGS. 4L-4M). These results indicate that elevated temperature selectively affects the recruitment of GBPL3 and several SA pathway-relevant Mediator complex subunits to the CBP60g promoter, independently of the NPR1-TGA1-CDK8 module (FIG. 5F).

### Example 9: CBP60g/SARD1 Expression is Rate-Limiting

The identification of CBP60g SARD1 transcription as the primary thermo-sensitive step in the SA pathway downstream of GBPL3 prompted us to ask if CBP60g SARD1 expression is a rate limiting step at elevated temperature for SA production and, if so, whether restoring CBP60g SARD1 expression would sufficiently render SA production resilient to heat wave. It was discovered here that, unlike expression of the activated SA receptor NPR1 or the SA biosynthetic gene ICS1 (FIG. 1D-E; FIGS. 3G-3J), 35S::CBP60g and 35S::SARD1 lines restored pathogen-induced SA production and maintained basal immunity to Pst DC3000 at 28° C., in contrast to Col-0 plants (FIGS. 7A-7C; FIGS. 8A-8D, FIGS. 9A-9E). Because CBP60g and SARD1 are functionally redundant (Wang, 2011), temperature-sensitive immunity to Pst DC3000 remained in the cbp60g single mutant, as expected (FIGS. 8E-8G).

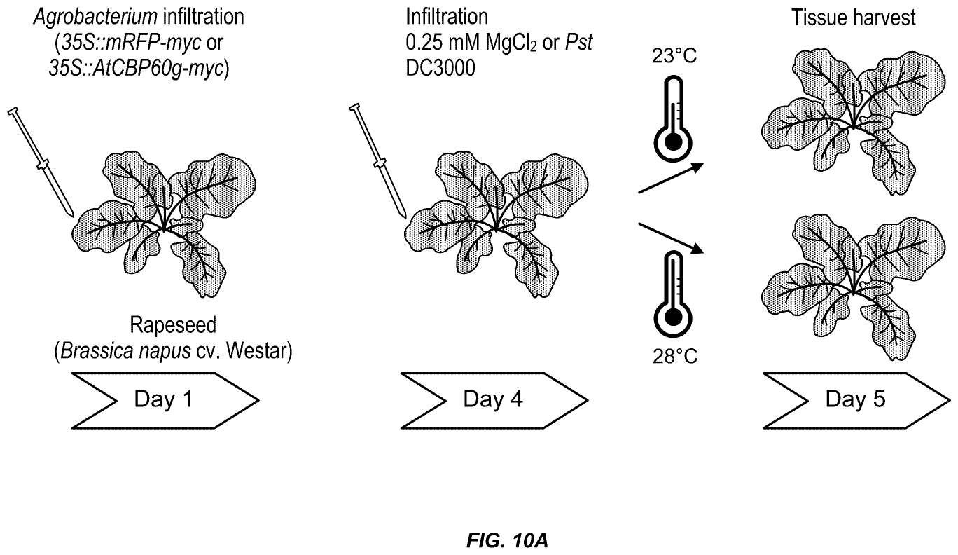

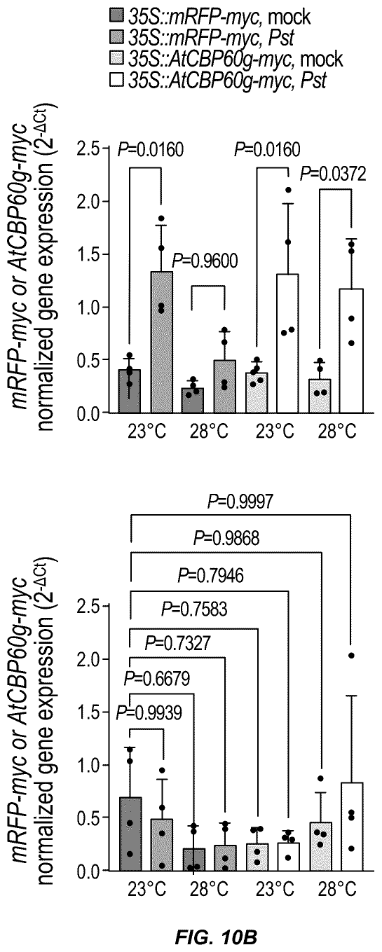

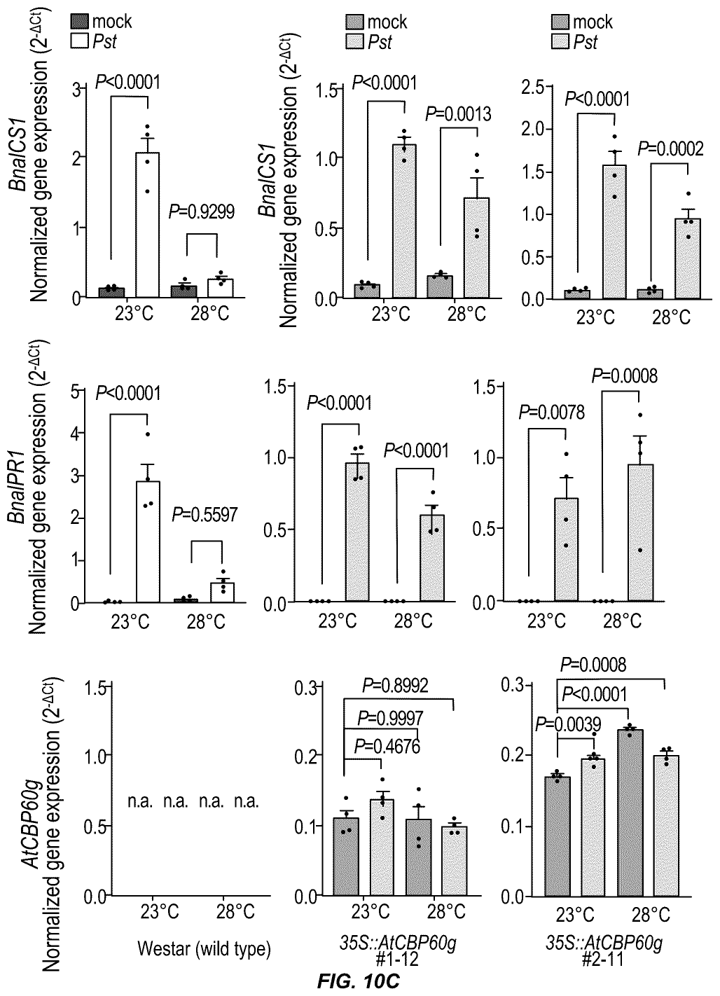

Surprisingly, in addition to restoring basal immunity to the virulent pathogen Pst DC3000, the temperature-resilient SA production and gene expression in 35S::CBP60g plants extends to infection by the nonpathogenic strain Pst ΔhrcC, which activates PTI in vivo (FIGS. 8H-8I), and to infection by ETI-activating Pst DC3000 (avrPphB) and Pst DC3000 (avrRps4) (FIGS. 7D-7E; FIGS. 8J-8K) (Zhou, 2020; Jones, 2016). Because ETI is widely used to guard crops against numerous pathogens and insects (Zhou 2020; Jones, 2016), these results suggest potentially broad applications of restoring CBP60g expression to counter suppression of not only basal immunity to virulent pathogens, but also ETI at elevated temperature. Finally, as shown in FIGS. 1A-1E and FIGS. 10B-10C, elevated temperature downregulated SA-response gene expression not only in Arabidopsis but also in crop plants such as tobacco and rapeseed. Both transient and stable AtCBP60g expression substantially restored Pst DC3000-induced expression of ICS1 and PRI orthologs (BnaICS1 and BnaPR1) in rapeseed leaves at elevated temperature (FIGS. 10A-10C).

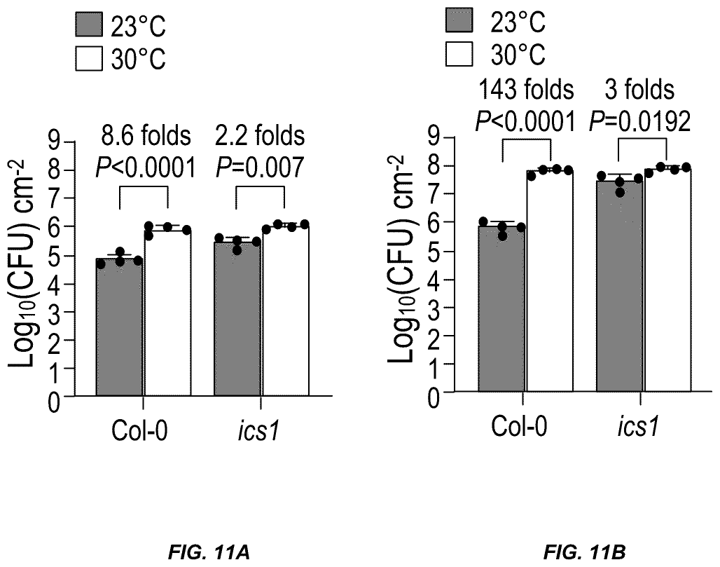

Consistent with their immune phenotypes, 35S::CBP60g Arabidopsis plants had restored pathogen-induced expression of CBP60g target genes ICS1, EDS1 and PAD4 at 28° C. (FIG. 8L). Further RNA-sequencing of pathogen-inoculated Col-0 and 35S::CBP60g plants at 23° C. and 28° C. found additional downregulated immunity genes also substantially restored in 35S::CBP60g plants (FIG. 7F; some data not shown). This included not only SA biosynthesis genes ICS1 and PBS3, but also pattern recognition receptor genes RLP23 and LYK5, PTI signaling gene MKK4, and pipecolic acid biosynthesis gene ALDI (FIG. 1I; some data not shown). Thus, 35S::CBP60g seems to safeguard other defense modules besides SA biosynthesis, consistent with previous observations that CBP60g is a master transcription factor regulating diverse sectors of the plant immune system (Sun, 2015). In line with this notion, SA-deficient ics1 (sid2-2) mutant plants still exhibit some temperature-sensitivity, albeit much less than wild-type Col-0 plants¹⁵ (FIGS. 11A-11B). It may partly explain why 35S::CBP60g plants (FIG. 3A-3F; some data not shown), but not 35S::ICS1 plants (FIGS. 3G-3H), can recover basal immunity at 28° C.

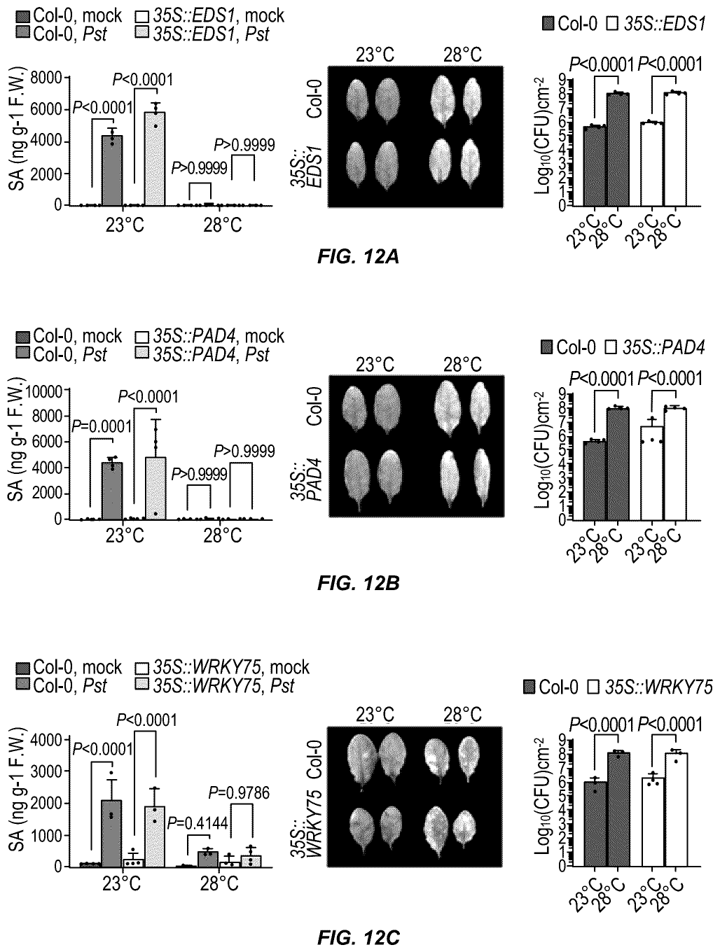

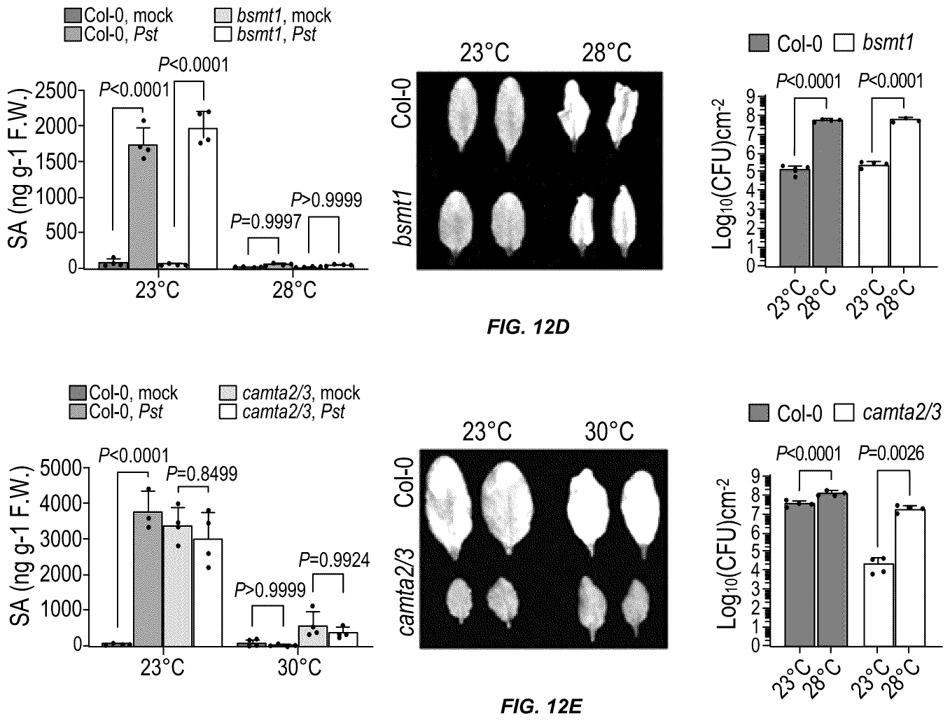

The inventors have surprisingly found here that restoration of SA production and immunity in 35S::CBP60g SARD1 plants appears to be unique among known SA pathway regulators. Constitutively expressing other elevated temperature-downregulated positive SA regulators, including ICS1, TGA1, EDS1, PAD4 and WRKY75328, all failed to restore SA production or basal immunity (FIGS. 3G-3H; FIG. 3N-3P; FIGS. 12A-12C). Similarly, loss-of-function mutations in heat-upregulated SA catabolic gene BSMT1 and SA transcriptional repressor genes CAMTA2/3 failed to restore SA levels and basal immunity at 28° C. (FIGS. 12D-12E). Additionally, we previously showed that gene mutations in JA, ABA or ethylene hormone pathway or DELLA-regulated PIFs, which are genetically antagonistic to the SA pathway, failed to revert SA suppression by elevated temperature (Huot, 2017a). These results illustrate that CBP60g/SARD1 are unique SA pathway regulators, the levels of which becomes rate-limiting for controlling ICS1-dependent and -independent immunity at elevated temperature.

### Example 10: Growth-Defense Trade-Off Optimization

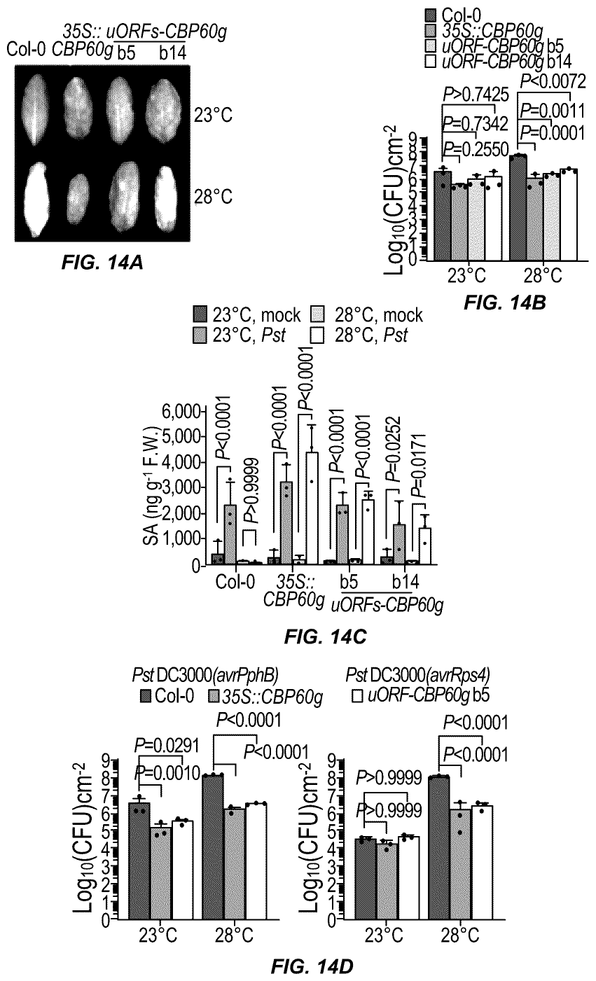

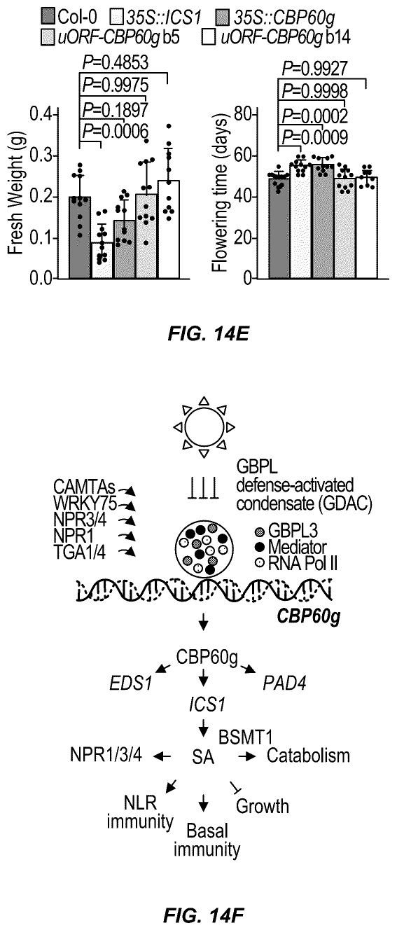

A common issue with increasing expression level of SA regulators is inhibition of plant growth and reproduction due to growth-defense tradeoff (Huot, 2014; Tan, 2020). This is illustrated with 35S::ICS1 plants, which have highly elevated basal SA levels at ambient temperature (FIG. 1D) and were characteristically dwarf (FIGS. 13A-13B). Interestingly, the growth of 35S::CBP60g and 35S::SARI1 plants was less adversely affected compared to 35S::ICS1 plants (FIG. 9E; FIGS. 13A-13B), consistent with low basal SA levels in 35S::CBP60g and 35S::SARD1 plants (FIG. 7A; FIG. 8B; FIG. 9A). Nevertheless, detailed characterization of 35S::CBP60g plants showed a delay in flowering (FIG. 13C). To minimize this developmental trade-off, we expressed CBP60g using the uORFs_{TBF1} strategy (FIG. 13D), which allows tightly controlled protein translation in response to pathogen infection (Xu, 2017). As shown in FIGS. 14A-14C, 35S::uORFs_{TBF1}-CBP60g plants maintained basal Pst DC3000 resistance and pathogen-induced SA production at 28° C. These plants also maintained substantial ETI against Pst DC3000 (avrPphB) and Pst DC3000 (avrRps4) at elevated temperature (FIG. 14D). Importantly, 35S::uORFs_{TBF1}-CBP60g plants showed normal growth and flowering time (FIG. 14E; FIG. 13A), demonstrating the promise of leveraging calibrated CBP60g expression to preserve plant immunity without significantly detrimental growth/developmental impacts.

### Example 11: Disease Resistance of 35S::AtCBP60g Transgenic Rapeseed Lines

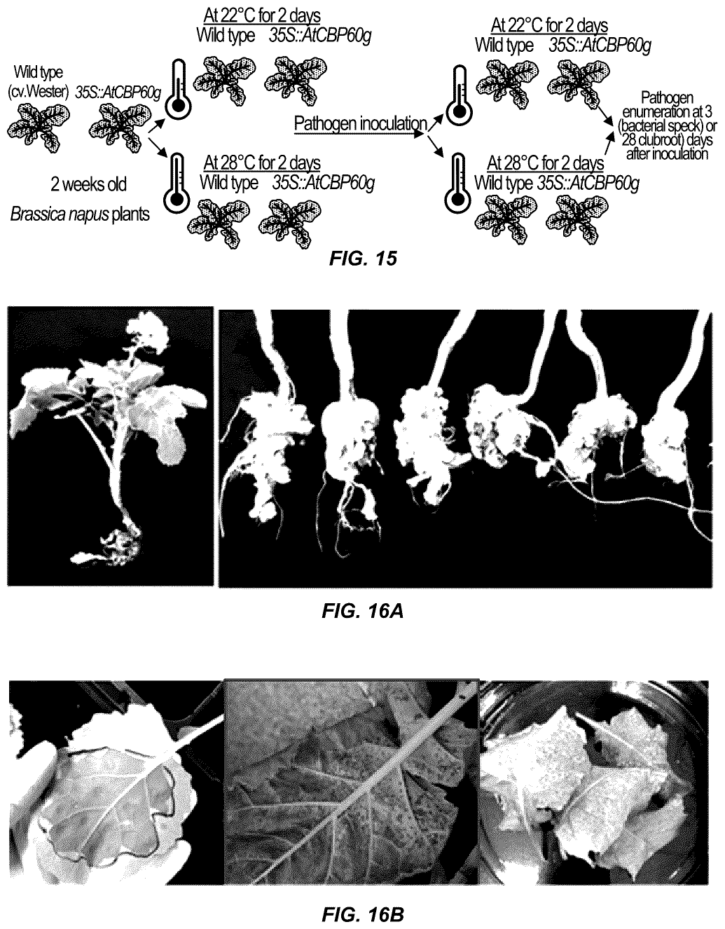

FIG. 15 depicts an experimental design for testing 35S::ArCBP60g transgenic rapeseed lines.

FIG. 16A shows the major symptoms in clubroot disease caused by the pathogen Plasmodiophora brassicae. FIG. 16B shows the major symptoms in bacterial speck disease caused by the pathogen Pseudomonas syringae pv. tomato DC3000.

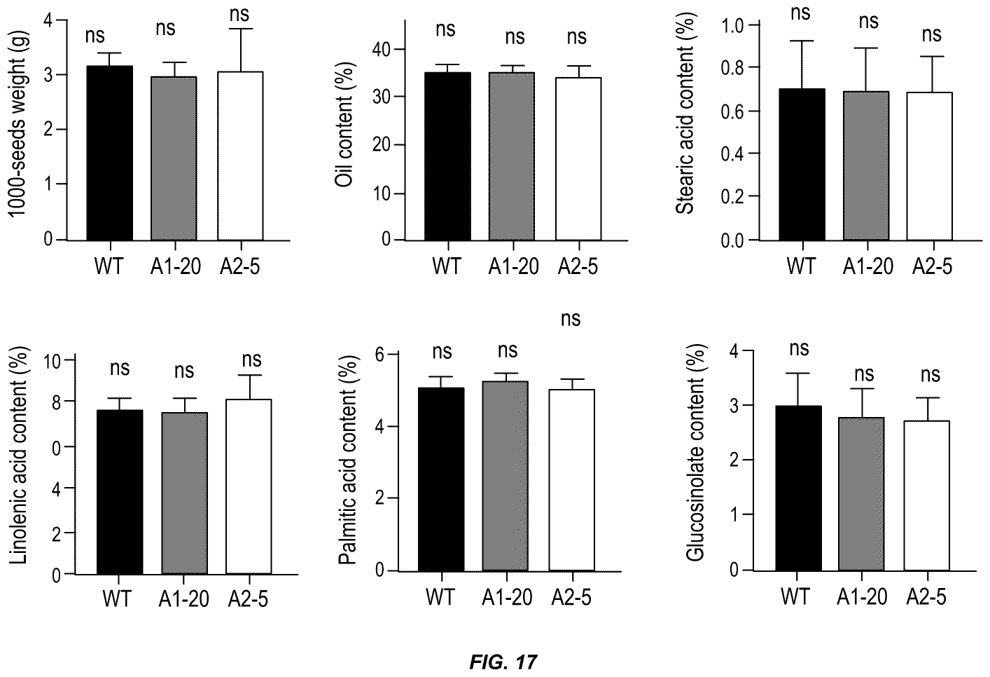

FIG. 17A shows the plant morphology of 35S::AtCBP60g transgenic rapeseed lines in cv. Westar (T3 generation). FIG. 17B shows the relative transcript levels of ArCBP60gin transgenic lines compared to that cv. Wester (control). FIG. 17C shows there was no difference in plant growth phenotype between the transgenic lines and Westar (control). Data analyzed by ANOVA with Tukey's HSD.

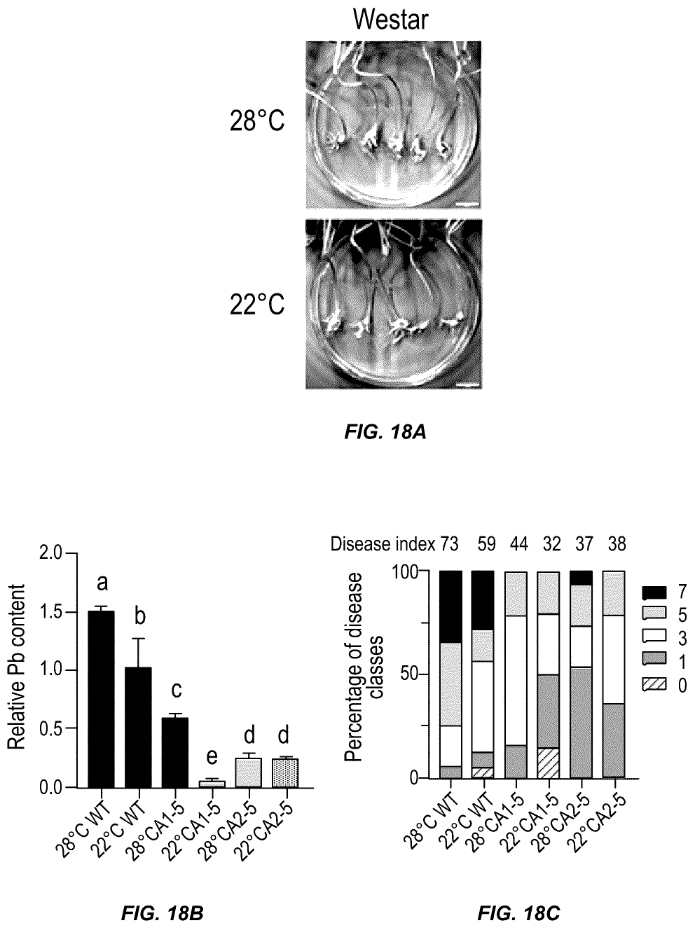

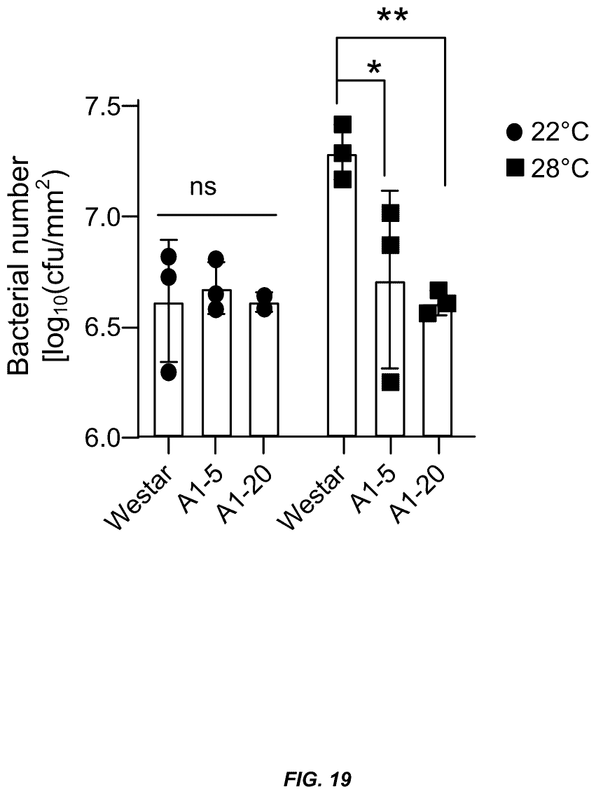

The panels of FIGS. 18A-18C show that there was no difference in other economic traits between 35S::ArCBP60g transgenic lines and cv. Wester (control). Data analyzed by ANOVA with Tukey's HSD.

35S::AtCBP60g transgenic plants have enhanced disease resistance to the clubroot pathogen Plasmodiophora brassicae (Pb). FIG. 18A shows that rapeseed has increased susceptibility to clubroot disease at warm temperature, as indicated by increased swollen roots. FIG. 18B shows 35S::ArCBP60g transgenic plants have increased resistance to clubroot-caused pathogen Pb at both normal and warm temperatures. Pb contents were normalized that in cv. Wester (WT) control plants at 22° C. Data analyzed by ANOVA with Tukey's HSD. FIG. 18C shows the disease index (percentage of plants showing any disease symptoms) is shown on the top of the column (n≥20).

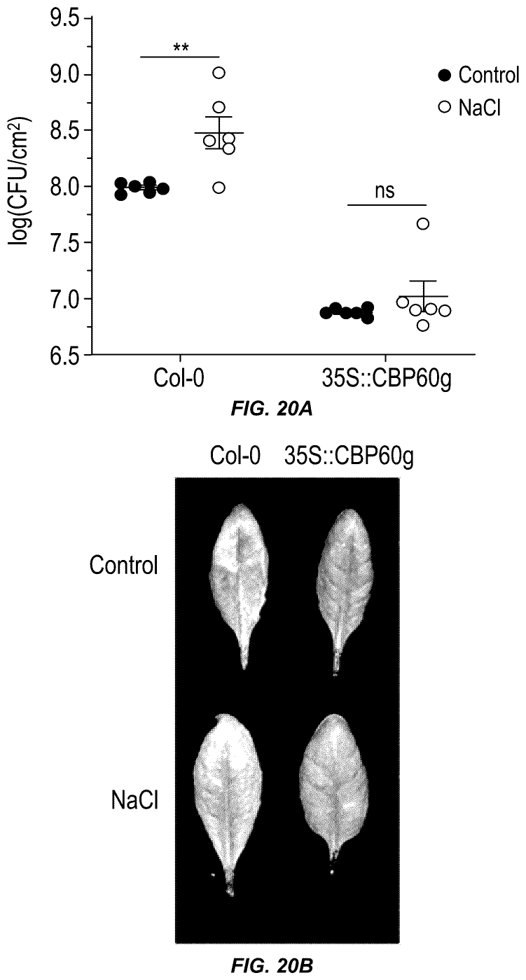

FIG. 20 shows that 35S::ArCBP60g transgenic plants have enhanced disease resistance to the bacterial speck pathogen Pseudomonas syringae pv. tomato DC3000 at warm temperature. Bacterial numbers in infected leaves of cv. Wester (control) and transgenic lines (A1-5 and A1-20) were enumerated. Data analyzed by ANOVA with Tukey's HSD.

### Example 12: Method to Screen Chemicals that Stabilize “GBPL3 Defense-Activated Biomolecular Condensates (GDACs)” at Warm Temperatures

Chemical libraries are screened by confocal laser scanning microscopy of Arabidopsis cells expressing a GBPL3-GFP fusion (35S::eGFP-GBPL3; Kim et al., PMID: 35768511), which allows for visualization of GDACs in living cells. Arabidopsis plants are sprayed with mock (0.1% DMSO) or benzo (1,2,3) thiadiazole-7-carbothioic acid-S-methyl ester (BTH; Chem Service, 100 μM, 0.1% DMSO). Plants are further incubated for 24 h at control (<23° C.) or warm temperature (>28° C.). Pre-treated leaves of 4- to 5-week-old 35S::eGFP-GBPL3 plants are imaged with an inverted Zeiss 880 single point scanning confocal attached to a fully motorized Zeiss Axio Observer microscope base, with Marzhauser linearly encoded stage and a 63× NA 1.4 oil plan apochromatic oil immersion objective lens. Images will be acquired by frame (line) scanning unidirectionally at 0.24 microseconds using the galvanometer-based imaging mode, with a voxel size of 0.22 μm×0.22 μm×1 μm and an area size of 224.92 μm 224.92 μm×1 μm μm in Zeiss Zen Black Acquisition software and saved as CZI files. Equal acquisition conditions (for example, excitation laser source intensity, range of acquired emission light range and exposure condition) are used for every image in each experiment. To maintain appropriate temperature during experiments, a portable temperature chamber and temperature-controlled specimen chamber of confocal microscope are used. To analyze images, FIJI/ImageJ software (Windows 64 1.52i version) is used.

### Example 13: 35S::CBP60g Plants are More Immune-Resilient Under Salt Stress Conditions

The inventors expanded upon the studies regarding the response of the transgenic plants to heat stress to examine their response to other forms of environmental stress associated with climate change. As temperatures rise and areas become more arid, there is also the tendency for the salinity of the ground water to rise as well. Using 35S::CBP60g transformed plants versus Arabidopsis Col-O controls, FIG. 20A shows P. syringae DC3000 bacterial levels at 3 days post inoculation (dpi). The bacterial population in wild-type Col-0 plants pretreated with 400 mM NaCl was significantly higher than that in wild-type Col-0 plants pretreated with water control. Data are mean≡s.e.m. (n=6 biological replicates) analyzed by two-way ANOVA with Tukey's HSD. The bacterial populations in 35S::CBP60g plants were much lower than those in wild-type Col-0 plants and there was no significant increase in bacterial population in 35S::CBP60g plants pretreated with 400 mM NaCl compared to 35S::CBP60g plants pretreated with water control. FIG. 20B shows pictures of leaves from P. syringae DC3000-inoculated wild-type Col-0 and 35S::CBP60g plants at 3 dpi.

One skilled in the art will readily appreciate that the present disclosure is well adapted to carry out the objects and obtain the ends and advantages mentioned, as well as those inherent therein. The present disclosure described herein are presently representative of representative embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

### REFERENCES

Attaran, E., et al. Methyl salicylate production and jasmonate signaling are not essential for systemic acquired resistance in Arabidopsis. Plant Cell. 21 954-971 (2009).
Chen, J., et al. NPR1 promotes its own and target gene expression in plant defense by recruiting CDK8. Plant Physiol. 181, 289-304 (2019).
Cheng, C., et al. Plant immune response to pathogens differs with changing temperatures. Nat. Commun. 4, 2530 (2013).
Clough, S. J., et al. Floral dip: a simplified method for Agrobacterium-mediated transformation of Arabidopsis thaliana. Plant J. 16, 735-743 (1998).
Dey, N., et al. Synthetic promoters in planta. Planta 242 1077-1094 (2015).
Ding, Y., et al. Opposite roles of salicylic acid receptors NPR1 and NPR3/NPR4 in transcriptional regulation of plant immunity. Cell 173, 1454-1467 (2018).
Fang, P., et al. Assessment of reference gene stability in Rice stripe virus and Rice black streaked dwarf virus infection rice by quantitative Real-time PCR. Virol. J. 12, 175 (2015).
Feys, B. J., et al. Arabidopsis SENESCENCE-ASSOCIATED GENE101 stabilizes and signals within an ENHANCED DISEASE SUSCEPTIBILITY1 complex in plant innate immunity. The Plant Cell. 17 2601-2613 (2005).
Hammoudi, V., et al. The Arabidopsis SUMO E3 ligase SIZ1 mediates the temperature dependent trade-off between plant immunity and growth. PLOS Genet. 14, e1007157 (2018).
Hinsch, M., et al. Identification of a new Arabidopsis disease resistance locus, RPS4, and cloning of the corresponding avirulence gene, avrRps4, from Pseudomonas syringae pv. Pisi. Mol. Plant Microbe Interact. 9, 55-61 (1996).
Huang, D. W., et al. Systematic and integrative analysis of large gene lists using DAVID bioinformatics resources. Nat. Protoc. 4, 44-57 (2009).
Huang, S., et al. A phase-separated nuclear GBPL circuit controls immunity in plants. Nature 594, 424-429 (2021).
Huot, B., et al. Growth-defense tradeoffs in plants: a balancing act to optimize fitness. Mol. Plant 7, 1267-1287 (2014).
Huot. B., et al. Dual impact of elevated temperature on plant defence and bacterial virulence in Arabidopsis. Nature Comm. 8:1808 nature.com/articles/s41467-017-01674-2 (2017).
Huot, B., et al. Dual impact of elevated temperature on plant defence and bacterial virulence in Arabidopsis. Nat. Commun. 8, 1808 (2017a).
Jain, M., et al. Validation of housekeeping genes as internal control for studying gene expression in rice by quantitative real-time PCR. Biochem. Biophys. Res. Commun. 345, 646-651 (2006).
Jones, M. A., et al. A Constitutively Active Allele of Phytochrome B Maintains Circadian Robustness in the Absence of Light. Plant Physiol. 169, 814-825 (2015).
Jones, J. D. G., et al. Intracellular innate immune surveillance devices in plants and animals. Science 354, aaf6395 (2016).
Jung, J. H., et al. Phytochromes function as thermosensors in Arabidopsis. Science 354, 886-889 (2016).
Jung, J. H. et al. A prion-like domain in ELF3 functions as a thermosensor in Arabidopsis. Nature 585, 256-260 (2020).
Katagiri, F., et al. The Arabidopsis thaliana-Pseudomonas syringae interaction. Arabidopsis Book 1, e0039 (2002).
Kim, Y., et al. Roles of CAMTA transcription factors and salicylic acid in configuring the low-temperature transcriptome and freezing tolerance of Arabidopsis. Plant J. 75, 364-376 (2013).
Kim, Y. S., et al. CAMTA-mediated regulation of salicylic acid immunity pathway genes in Arabidopsis exposed to low temperature and pathogen infection. Plant Cell. 29, 2465-2477 (2017).
Kim, Y., et al. Arabidopsis CAMTA transcription factors regulate pipecolic acid biosynthesis and priming of immunity genes. Mol. Plant 13, 157-168 (2020).
Kusajima, M., et al. Abscisic acid modulates salicylic acid biosynthesis for systemic acquired resistance in tomato. Biosci. Biotechnol. Biochem. 81, 1850-1853 (2017).
Legris, M., et al. Phytochrome B integrates light and temperature signals in Arabidopsis. Science 354, 897-900 (2016).
Mang, H. G., et al. Abscisic acid deficiency antagonizes high-temperature inhibition of disease resistance through enhancing nuclear accumulation of resistance proteins SNC1 and RPS4 in Arabidopsis. Plant Cell 24, 1271-1284 (2012).
Mou, Z., et al. Inducers of plant systemic acquired resistance regulate NPR1 function through redox changes. Cell 113, 935-944 (2003).
Pajerowska-Mukhtar, K. M., et al. The HSF-like transcription factor TBF1 is a major molecular switch for plant growth-to-defense transition. Curr. Biol. 22, 103-112 (2012).
Peng, Y., et al. Salicylic acid: biosynthesis and signaling. Annu. Rev. Plant Biol. 72, 761-791 (2021).
Pombo, M. A., et al. Use of RNA-seq data to identify and validate RT-qPCR reference genes for studying the tomato-Pseudomonas pathosystem. Sci. Rep. 7, 44905 (2017).
Saleh, A., et al. Posttranslational modifications of the master transcriptional regulator NPR1 enable dynamic but tight control of plant immune responses. Cell Host Microbe 18, 169-182 (2015).
Samaradivakara, S. P., et al. Overexpression of NDR1 leads to pathogen resistance at elevated temperatures. New Phytol. doi.org/10.1111/nph.18190 (2022).
Simonich, M. T., et al. A disease resistance gene in Arabidopsis with specificity for the avrPph3 gene of Pseudomonas syringae pv. phaseolicola. Mol. Plant Microbe Interact. 8, 637-640 (1995).
Sun, T., et al. ChIP-seq reveals broad roles of SARD1 and CBP60g in regulating plant immunity. Nat. Commun. 6, 10159 (2015).
Tan, S., et al. Salicylic acid targets protein phosphatase 2A to attenuate growth in plants. Curr. Biol. 30, 381-395.e8 (2020).
Villao-Uzho, L., et al. Plant promoters: their identification, characterization, and role in gene regulation. Genes 14, 1226. Doi.org/10.3390/genes14061226 (2023).
Wan, D., et al. Calmodulin-binding protein CBP60g is a positive regulator of both disease resistance and drought tolerance in Arabidopsis. Plant Cell Rep. 31, 1269-1281 (2012).
Wang, Y. Analysis of temperature modulation of plant defense against biotrophic microbes. Mol. Plant Microbe Interact. 22, 498-506 (2009).
Wang, L., et al. Arabidopsis CaM binding protein CBP60g contributes to MAMP-induced SA accumulation and is involved in disease resistance against Pseudomonas syringae. PLOS Pathog. 5, e1000301 (2009a)
Wang, L., et al. CBP60g and SARD1 play partially redundant critical roles in salicylic acid signaling. Plant J. 67, 1029-1041 (2011).
Wang, C., et al. The Arabidopsis Mediator complex subunit16 is a key component of basal resistance against the necrotrophic fungal pathogen Sclerotinia sclerotiorum. Plant Physiol. 169, 856-872 (2015).
Wildermuth, M. C., et al. Isochorismate synthase is required to synthesize salicylic acid for plant defence. Nature 414, 562-565 (2001).
Xing, D., et al. Effects of mutations and constitutive overexpression of EDS1 and PAD4 on plant resistance to different types of microbial pathogens. Plant Sci. 171, 251-262 (2006).
Xu, G., et al. uORF-mediated translation allows engineered plant disease resistance without fitness costs. Nature 545, 491-494 (2017).
Yamaguchi, N., et al. Protocols: Chromatin immunoprecipitation from Arabidopsis tissues. Arabidopsis Book 12, e0170 (2014).
Yan, J., et al. Cell autonomous and non-autonomous functions of plant intracellular immune receptors in stomatal defense and apoplastic defense. PLOS Pathog. 15, e1008094 (2019).
Zhang, Y., et al. Control of salicylic acid synthesis and systemic acquired resistance by two members of a plant-specific family of transcription factors. Proc. Natl Acad. Sci. USA 107, 18220-18225 (2010).
Zhang, X., et al. The Arabidopsis mediator complex subunit 16 positively regulates salicylate-mediated systemic acquired resistance and jasmonate/ethylene-induced defense pathways. Plant Cell 24, 4294-4309 (2012).
Zhang, L., et al. Transcription factor WRKY75 interacts with DELLA proteins to affect flowering. Plant Physiol. 176, 790-803 (2018).
Zhou, Y., et al. Control of petal and pollen development by the plant cyclin-dependent kinase inhibitor ICK1 in transgenic Brassica plants. Planta 215, 248-257 (2002).
Zhou, J.-M., et al. Plant immunity: danger perception and signaling. Cell 181, 978-989 (2020).
Zhu, Y., et al. Temperature modulates plant defense responses through NB-LRR proteins. PLOS Pathog. 6, e1000844 (2010).
Zhu, Y., et al. CYCLIN-DEPENDENT KINASE 8 differentially regulates plant immunity to fungal pathogens through kinase-dependent and -independent functions in Arabidopsis. Plant Cell 26, 4149-4170 (2014).

1. A plant comprising a genome having inserted into a genomic site thereof a constitutive promoter operably linked to a coding sequence comprising a pathogen-responsive upstream open reading frame (uORF) element and a polynucleotide sequence encoding a CPB60 protein, wherein the plant maintains salicylic acid (SA)-mediated immunity to a pathogen when under an environmental stress.

2. The plant of claim 1, wherein the polynucleotide sequence encoding a CPB60 protein is an exogenous polynucleotide sequence.

3. The plant of claim 1, wherein the CPB60 protein is Arabidopsis thaliana CPB60g protein, Brachpodium dystachion CPB60g protein, Sorghum bicolor CPB60g protein, Zea mays CPB60g protein, Hordeum vulgare CPB60g protein, Triticum aestivum CPB60g protein, Solamum lycopersicum CPB60g protein, Solamum tuberosum CPB60g protein, Brassica napus CPB60g protein, Gossypium hirsutum CPB60g protein, Glycine max CPB60g protein, Medicago truncatula CPB60g protein, or an ortholog thereof.

4. The plant of claim 1, wherein the CPB60 protein has at least 90% identity to any one of SEQ ID NOs: 43-45.

5. The plant of claim 1, wherein the pathogen-responsive uORF element comprises at least one uORF sequence operably linked to an immune-responsive promoter.

6. The plant of claim 5, wherein the pathogen-responsive uORF element comprises one or two Arabidopsis thaliana TL1-BINDING TRANSCRIPTION FACTOR 1 (AITBF1) uORF sequences.

7. The plant of claim 1, wherein the constitutive promoter is a plant promoter, a viral promoter, or a Cauliflower Mosaic Virus (CMV) 35S promoter.

8. (canceled)

9. (canceled)

10. (canceled)

11. The plant of claim 5, wherein the pathogen-responsive uORF comprises a sequence having at least 90% identity to SEQ ID NO: 61.

12. (canceled)

13. The plant of claim 1, wherein the environmental stress is at least one of heat stress, drought, or salinity.

14. The plant of claim 13, wherein the heat stress is an ambient temperature that is elevated over a maximum ambient temperature for the plant.

15. The plant of claim 1, wherein the pathogen is at least one of a fungus, a fungal-like organism, a bacterium, a phytoplasma, a protist, a virus, or a viroid.

16. (canceled)

17. (canceled)

18. (canceled)

19. (canceled)

20. A plant comprising a progeny of the stably transfected plant of claim 1.

21. A method for making a plant that maintains salicylic acid (SA)-mediated immunity to a pathogen under an environmental stress, the method comprising:
introducing at a genomic site in a genome of a plant of interest a nucleic acid comprising a constitutive promoter operably linked to a coding sequence comprising a pathogen-responsive upstream open reading frame (uORF) element and a polynucleotide sequence encoding a CPB60 protein.

22-35. (canceled)

36. The method of claim 21, wherein the step of introducing nucleic acid into the plant of interest comprises Agrobacterium-mediated transfection.

37. The method of claim 21, wherein the genomic site is a location of an endogenous CPB60 gene in the genome of the plant of interest, and wherein the step of introducing the nucleic acid into the plant of interest results in replacement of all or a portion of the endogenous CPB60 gene in the genome of the plant of interest with the nucleic acid sequence such that the endogenous CPB60 gene is not expressed in the plant of interest.

38. A method for screening a subject plant for increased immunity to a pathogen when under an environmental stress, the method comprising:
a) incubating a first copy of the subject plant with the pathogen under normal environmental growing conditions for a defined time period, wherein the subject plant comprises a plant made according to the method of claim 21;
b) incubating a first copy of a control plant with the pathogen under the normal environmental growing conditions for the defined time period, wherein the control plants is the same species as the plant of interest of claim 21 and is susceptible to the pathogen under the environmental stress;
c) incubating a second copy of the subject plant with the pathogen under conditions comprising the environmental stress for the defined time period;
d) incubating a second copy of the control plant with the pathogen under conditions comprising the environmental stress for the defined time period;
e) assessing the first and second copies of the subject plant and the first and second copies of the control plant for characteristics of exposure to the pathogen at the end of the defined time period; and
f) indicating that the subject plant has (i) increased immunity to a pathogen when under an environmental stress if the second copy of the subject plant exhibits fewer and/or reduced characteristics of exposure to the pathogen after the defined time period as compared to the second copy of the control plant after the defined time period, or (ii) does not have increased immunity to a pathogen when under an environmental stress if the second copy of the subject plant exhibits similar characteristics of exposure to the pathogen after the defined time period as compared to the second copy of the control plant after the defined time period.

39. The method of claim 38, wherein the second subject plant having increased immunity to a pathogen when under an environmental stress exhibits similar characteristics of exposure to the pathogen after the defined time period to the first copy of the subject plant and/or the first copy of the control plant after the defined time period.

40. The method of claim 38, wherein the environmental stress is at least one of heat stress, drought, or salinity.

41. The method of claim 40, wherein the heat stress is an ambient temperature that is elevated over a maximum ambient temperature for the plant.

42. The method of claim 38, wherein the pathogen is selected from the group consisting of a fungus, a fungal-like organism, a bacterium, a phytoplasma, a protist, a virus, and a viroid.
